(19) United States
(12) Patent Application Publication
(10) Pub. No.: US 2011/0178064 A1
(43) Pub. Date: Jul. 21, 2011

(54) SPIRO CONDENSED BARBITURIC ACID DERIVATIVES FOR USE AS ANTIBACTERIAL

(75) Inventors: Gregory Steven Basarab, Waltham, MA (US); Jacques Dumas, Waltham, MA (US); Pamela Hill, Waltham, MA (US)

(73) Assignee: ASTRAZENECA R&D, Sodertalje (SE)

(21) Appl. No.: 12/669,282

(22) PCT Filed: Jul. 16, 2008
(86) PCT No.: PCT/GB2008/050581

§ 371 Date: Feb. 3, 2011

Related U.S. Application Data
(60) Provisional application No. 60/950,114, filed on Jul. 16, 2007.

(51) Int. Cl. A61K 31/55; C07D 239/00; C07D 223/00; A61K 31/527; A61P 31/04

(52) U.S. Cl. 514/212.02; 544/231; 540/543; 514/257

(57) ABSTRACT

In one aspect, the present invention relates to compounds of Formula (I): to pharmaceutically acceptable salts thereof, to methods of using them to treat bacterial infections, and to method for their preparation.

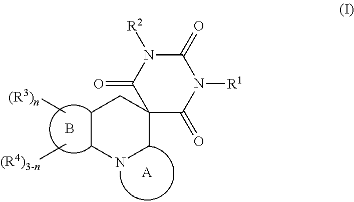

The present invention relates to novel substituted heterocycles, their pharmaceutical compositions and methods of use. In addition, the present invention relates to therapeutic methods for the treatment of bacterial infections.

The international microbiological and infectious disease community continues to express serious concern that the continuing evolution of antibacterial resistance could result in bacterial strains against which currently available antibacterial agents will be ineffective. The outcome of such an occurrence could have considerable morbidity and mortality. In general, bacterial pathogens may be classified as either Gram-positive or Gram-negative pathogens. Antibiotic compounds with effective activity against both Gram-positive and Gram-negative pathogens are generally regarded as having a broad spectrum of activity.

Gram-positive pathogens are of particular concern because of the development of resistant strains that are both difficult to treat and difficult to eradicate from the hospital environment once established. Examples of such strains are methicillin resistant Staphylococcus aureus (MRSA), methicillin resistant coagulase-negative staphylococci (MRCNS), penicillin resistant Streptococcus pneumoniae and multiple resistant Enterococcus faecium. Resistance is increasing at a steady rate rendering many agents less effective in the treatment of Gram-positive pathogens. In addition, there is increasing resistance to agents such as β-lactams, quinolones and macrolides used for the treatment of upper respiratory tract infections caused by Gram-negative strains including H. influenzae and M. catarrhalis. In addition, nosocomial Gram-negative pathogens, such as Pseudomonas aeruginosa, are difficult to treat due to resistance development. Consequently, in order to overcome the threat of widespread multi-drug resistant organisms, there is an on-going need to develop new antibacterials.

Deoxyribonucleic acid (DNA) gyrase is a member of the type II family of topoisomerases that control the topological state of DNA in cells (Champoux, J. J.; 2001. Ann. Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

Antibacterials targeting DNA gyrase are well established in the art, including examples such as the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated primarily by mutations in the target (DNA gyrase and topoisomerase IV) is becoming an increasing problem in several organisms, including S. aureus and Streptococcus pneumoniae (Hooper, D. C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

The present invention relates to compounds of Formula (I):

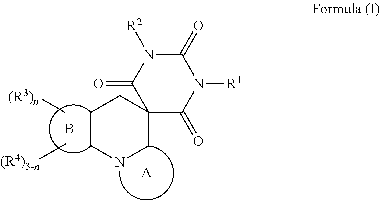

and to pharmaceutically acceptable salts thereof, wherein:
Ring A is a 5- to 7-membered non-aromatic heterocyclic ring, wherein
1) said 5- to 7-membered non-aromatic heterocyclic ring optionally contains, in addition to the nitrogen, a member selected from —O—, —NH—, —S—, —S(O)—, and —S(O)₂—;
2) said 5- to 7-membered non-aromatic heterocyclic ring is optionally substituted on carbon with one or more R⁷;
3) two R⁷ substituents on one carbon atom may together optionally form the group ═O or the group ═N(OR^{7a}); and
4) any —NH— moiety said 5- to 7-membered heterocyclic ring is optionally substituted with R⁷*;
   Ring B is a 5- or 6-membered aromatic heterocyclic ring;
   n is 0 to 3;
   R¹ is selected from H, C₁₋₆alkyl, carbocyclyl, heterocyclyl, —C(O)—H, —C(O)—R^{1b}, —C(O)₂R^{1c}, —C(O)—N(R^{1a})₂, —S(O)—R^{1b}, —S(O)₂—R^{1b}, —S(O)₂—N(R^{1a})₂, —C(R^{1a})═N—R^{1a}, and —C(R^{1a})═N—OR^{1a}, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
   R^{1a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
   R^{1b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein any —NH— moiety of said heterocyclyl is optionally substituted with R¹⁰*;
   R^{1c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
   R² is selected from H, C₁₋₆alkyl, carbocyclyl, heterocyclyl, —C(O)—H, —C(O)—R^{2b}, —C(O)₂R^{2c}, —C(O)—N(R^{2a})₂, —S(O)—R^{2b}, —S(O)₂—R^{2b}, —S(O)₂—N(R^{2a})₂, —C(R^{2a})═N═R^{2a}, and —C(R^{2a})═N—OR^{2a}, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
   R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
   R^{2b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein any —NH— moiety of said heterocyclyl is optionally substituted with R²⁰*;
   R^{2c} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
   R³ in each occurrence is independently selected from —X—R⁵, —W—R⁶, —C(O)—N(R^{3a})—S(O)₂—R^{3b}, —C(R^{3a})═N—R^{3y}, —C(R^{3a})═N—NR^{3a}—C(O)—R^{3b}, —C(N(R^{3a})₂)═N—R^{3y}, —C(N(R^{3a})₂)═N—OR^{3y}, —C(N(R^{3a})₂)═N—C(O)—R^{3b}, —C(N(R^{3a})₂)═N—S(O)₂—R^{3b}, —C(N(R^{3a})₂)═N—CN, —N═C(R^{3y})₂, —N(R^{3a})—S(O)₂—N(R^{3y})₂, —N(R^{3a})—N(R^{3y})₂, —N(R^{3a})—C(O)—N(R^{3y})₂, —N(R^{3a})—C(O)—N(R^{3a})—S(O)₂—R^{3b}, —N(R^{3a})—C(R^{3a})═N(R^{3y}), —N(R^{3a})—C(R^{3a})═N—OR^{3y}, —N(R^{3a})—C(R^{3a})═N—C(O)—R^{3b}, —N(R^{3a})—C(R^{3a})═N—S(O)₂R^{3b}, —N(R^{3a})—C(R^{3a})═N—CN, —N(R^{3a})—C(N(R^{3a})₂)═N—R^{3y}, —N(R^{3a})—C(N(R^{3a})₂)═N—OR^{3y}, —N(R^{3a})—C(N(R^{3a})₂)═N—C(O)—R^{3b}, —N(R^{3a})—C(N(R^{3a})₂)═N—S(O)₂—R^{3b}, —N(R^{3a})—C(N(R^{3a})₂)═N—CN, —O—C(O)—R^{3b}, and —Si(R^{3b})₃;
   R^{3a} and R^{3y} in each occurrence are independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein any —NH— moiety of said heterocyclyl is optionally substituted with R³⁰*;
   R^{3b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein any —NH— moiety of said heterocyclyl is optionally substituted with R³⁰*;
   R⁴ in each occurrence is independently selected from H, halo, —CN, C₁₋₆alkyl, C₂₋₆alkenyl, C₂₋₆alkynyl, carbocyclyl, heterocyclyl, —OR^{4d}, —SR^{4d}, —N(R^{4d})₂, —N(R^{4a})—C(O)—R^{4e}, —NO₂, —C(O)—H, —C(O)—R^{4e}, —C(O)₂R^{4d}, —C(O)—N(R^{4d})₂, —O—C(O)—N(R^{4d})₂, —N(R^{4a})—C(O)₂R^{4d}, —S(O)—R^{4e}, —S(O)₂—R^{4e}, —S(O)₂—N(R^{4d})₂, —N(R^{4a})—S(O)₂—R^{4e}, and —C(R^{4a})═N—OR^{4d}, wherein said C₁₋₆alkyl, C₂₋₆alkenyl, and C₂₋₆alkynyl in each occurrence are optionally and independently substituted with one or more R^{40x}, and wherein said carbocyclyl and heterocyclyl in each occurrence are optionally and independently substituted on carbon with one or more R⁴⁰, and wherein any —NH— moiety of said heterocyclyl is optionally substituted with R⁴⁰*;
   R^{4a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁴⁰, and wherein any —NH— moiety of said heterocyclyl is optionally substituted with R⁴⁰*;
   R^{4d} in each occurrence is independently selected from H, C₁₋₆alkyl, carbocyclyl, and aromatic heterocyclyl, wherein said C₁₋₆alkyl, carbocyclyl, and aromatic heterocyclyl in each occurrence are optionally and independently substituted on carbon with one or more R⁴⁰, and wherein any —NH— moiety of said aromatic heterocyclyl is optionally substituted with R⁴⁰*;
   R^{4e} in each occurrence is selected from C₁₋₆alkyl, C₂₋₆alkenyl, C₂₋₆alkynyl, carbocyclyl, and aromatic heterocyclyl, wherein said C₁₋₆alkyl, C₂₋₆alkenyl, C₂₋₆alkynyl, carbocyclyl, and aromatic heterocyclyl in each occurrence are optionally and independently substituted on carbon with one or more R⁴⁰, and wherein any —NH— moiety of said aromatic heterocyclyl is optionally substituted with R⁴⁰*;
   R⁵ is selected from heterocyclyl and —Si(R^{5b})₃, wherein said heterocyclyl is optionally substituted on carbon with one or more R⁵⁰, and wherein any —NH— moiety of said heterocyclyl is optionally substituted with R⁵⁰*;
   R^{5b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁴⁰, and wherein any —NH— moiety of said heterocyclyl is optionally substituted with R⁵⁰*;

R⁶ is non-aromatic heterocyclyl, wherein said non-aromatic heterocyclyl is optionally substituted on carbon with one or more R⁶⁰, and wherein any —NH— moiety of said non-aromatic heterocyclyl is optionally and independently substituted with R⁶⁰*;

R⁷ is selected from halo, —CN, C₁₋₆alkyl, C₂₋₆alkenyl, C₂₋₆alkynyl, carbocyclyl, heterocyclyl, —OR^{7a}, —SR^{7a}, —N(R^{7a})₂, —N(R^{7a})—C(O)—R^{7b}, —N(R^{7a})—N(R^{7a})₂, —NO₂, —C(O)—H, —C(O)R^{7b}, —C(O)₂R^{7a}, —C(O)—N(R^{7a})₂, —O—C(O)—N(R^{7a})₂, —N(R^{7a})—C(O)₂R^{7a}, —N(R^{7a})—C(O)—N(R^{7a})₂, —O—C(O)—R^{7b}, —S(O)—R^{7b}, —S(O)₂—R^{7b}, —S(O)₂—N(R^{7a})₂, —N(R^{7a})—S(O)₂—R^{7b}, —C(R^{7a})═N—R^{7a}, and —C(R^{7a})═N—OR^{7a}, wherein said C₁₋₆alkyl, C₂₋₆alkenyl, C₂₋₆alkynyl, carbocyclyl, and heterocyclyl are optionally substituted on carbon with one or more R⁷⁰, and wherein any —NH— moiety of said heterocyclyl is optionally substituted with R⁷⁰*;
R⁷* in each occurrence is independently selected from C₁₋₆alkyl, carbocyclyl, heterocyclyl, —C(O)—H, —C(O)—R^{7b}, —C(O)₂R^{7c}, —C(O)—N(R^{7a})₂, —S(O)—R^{7b}, —S(O)₂—R^{7b}, —S(O)₂—N(R^{7a})₂, —C(R^{7a})═N—R^{7a}, and —C(R^{7a})═N—OR^{7a}, wherein said C₁₋₆alkyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
R^{7a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
R^{7b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
R^{7c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
R¹⁰ in each occurrence is independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})—C(O)—R^{10b}, —N(R^{10a})—N(R^{10a})₂, —NO₂, —C(O)—H, —C(O)—R^{10b}, —C(O)₂R^{10a}, —C(O)—N(R^{10a})₂, —O—C(O)—N(R^{1a})₂, —N(R^{10a})—C(O)₂R^{10a}, —N(R^{10a})—C(O)—N(R^{10a})₂), —O—C(O)—R^{10b}, —S(O)—R^{10b}, —S(O)₂—R^{10b}, —S(O)₂—N(R^{10a})₂, —N(R^{10a})—S(O)₂—R^{10b}, —C(R^{10a})═N—R^{10a}, and —C(R^{10a})═N—OR^{10a}, wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein any —NH— moiety of said heterocyclyl is optionally substituted with R^{a}*;
R¹⁰* in each occurrence is independently selected from C₁₋₆alkyl, carbocyclyl, heterocyclyl, —C(O)—H, —C(O)—R^{10b}, —C(O)₂R^{10c}, —C(O)—N(R^{10a})₂, —S(O)R^{10b}, —S(O)₂R^{10b}, —S(O)₂—N(R^{10a})₂, —C(R^{10a})═N—R^{10a}, and —C(R^{10a})═N—OR^{10a}, wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R²⁰ in each occurrence is independently selected from halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, —N(R^{20a})—C(O)—R^{20b}, —N(R^{20a})—N(R^{20a})₂), —NO₂, —C(O)—H, —C(O)—R^{20b}, —C(O)₂R^{20a}, —C(O)—N(R^{20a})₂, —O—C(O)—N(R^{20a})₂, —N(R^{20a})—C(O)₂R^{20a}, —N(R^{20a})—C(O)—N(R^{20a})₂, —O—C(O)—R^{20b}), —S(O)—R^{20b}), —S(O)₂R^{20b}, —S(O)₂—N(R^{20a})₂, —N(R^{20a})—S(O)₂R^{20b}, —C(R^{20a})═N—R^{20a}, and —C(R^{20a})═N—OR^{20a}, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R²⁰* in each occurrence is independently selected from C₁₋₆alkyl, carbocyclyl, heterocyclyl, —C(O)—H, —C(O)—R^{20b}, —C(O)₂R^{20c}, —C(O)—N(R^{20a})₂, —S(O)—R^{20b}, —S(O)₂—R^{20b}, —S(O)₂—N(R^{20a})₂, —C(R^{20a})═N—R^{20a}, and —C(R^{20a})═N—OR^{20a}, wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20c} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})—C(O)—R^{30b}, —N(R^{30a})—N(R^{30a})₂, —NO₂, —C(O)—H, —C(O)—R^{30b}, —C(O)₂R^{30a}, —C(O)—N(R^{30a})₂, —O—C(O)—N(R^{30a})₂, —N(R^{30a})—C(O)₂R^{30a}, —N(R^{30a})—C(O)—N(R^{30a})₂, —O—C(O)—R^{30B}, —S(O)—R^{30b}, —S(O)₂—R^{30b}, —S(O)₂—N(R^{30a})₂, —N(R^{30a})—S(O)₂—R^{30b}, —Si(R^{30b})₃, —C(R^{30a})═N—R^{30a}, and —C(R^{30a})═N—OR^{30a}, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein any —NH— moiety of said heterocyclyl is optionally substituted with R^{c}*;
R³⁰* in each occurrence is independently selected from C₁₋₆alkyl, carbocyclyl, heterocyclyl, —C(O)—H, —C(O)—R^{30b}, —C(O)₂R^{30c}, —C(O)—N(R^{30a})₂, —S(O)—R^{30b}, —S(O)₂—R^{30b}, —S(O)₂—N(R^{30a})₂, —C(R^{30a})═N—R^{30a}, and —C(R^{30a})═N—OR^{30a}, wherein said C₁₋₆alkyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30c} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein any —NH— moiety of said heterocyclyl is optionally substituted with R^{c}*;
R⁴⁰ in each occurrence is independently selected from halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})—C(O)—R^{40b}, —N(R^{40a})—N(R^{40a})₂, —NO₂, —C(O)—H, —C(O)—R^{40b}, —C(O)₂R^{40a}, —C(O)—N(R^{40a})₂, —O—C(O)—N(R^{40a})₂, —N(R^{40a})—C(O)₂R^{40a}, —N(R^{40a})—C(O)—N(R^{40a})₂, —O—C(O)—R^{40b}, —S(O)—R^{40b}, —S(O)₂—R^{40b}, —S(O)₂—N(R^{40a})₂, —N(R^{40a})—S(O)₂—R^{40b}, —C(R^{40a})═N—R^{40a}, and —C(R^{40a})═N—OR^{40a}, wherein said C₂₋₆alkenyl, C₂₋₆alkynyl, carbocyclyl, and heterocyclyl in each occurrence are optionally and independently substituted on carbon with one or more R^{d}, and wherein any —NH— moiety of said heterocyclyl is optionally substituted with R^{d}*;
R⁴⁰* in each occurrence is independently selected from C₁₋₆alkyl, carbocyclyl, heterocyclyl, —C(O)—H, —C(O)—R^{40b}, —C(O)₂R^{40c}, —C(O)—N(R^{40a})₂, —S(O)—R^{40b}, —S(O)₂—R^{40b}, —S(O)₂—N(R^{40a})₂, —C(R^{40a})═N—R^{40a}, and —C(R^{40a})═N—OR^{40a}, wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40x} in each occurrence is independently selected from halo, —CN, C₁₋₆alkyl, C₂₋₆alkenyl, C₂₋₆alkynyl, carbocyclyl, —OR^{40a}, —SR^{40a}, —N(R^{40a})₂, —N(R^{40a})—C(O)—R^{40b}, —N(R^{40a})—N(R^{40a})₂, —NO₂, —C(O)—H, —C(O)—R^{40b}, —C(O)₂R^{40a}, —C(O)—N(R^{40a})₂, —O—C(O)—N(R^{40a})₂, —N(R^{40a})—C(O)₂R^{40a}, —N(R^{40a})—C(O)—N(R^{40a})₂, —O—C(O)—R^{40b}, —S(O)—R^{40b}, —S(O)₂—R^{40b}, —S(O)₂—N(R^{40a})₂, —N(R^{40a})—S(O)₂—R^{40b}, —C(R^{40a})═N—R^{40a}, and —C(R^{40a})═N—OR^{4a}, wherein said C₁₋₆alkyl, C₂₋₆alkenyl, C₂₋₆alkynyl, and carbocyclyl in each occurrence are optionally and independently substituted on carbon with one or more R^{d};
R⁵⁰ in each occurrence is independently selected from halo, —CN, C₁₋₆alkyl, C₂₋₆alkenyl, C₂₋₆alkynyl, carbocyclyl, heterocyclyl, —OR^{50a}, —SR^{50a}, —N(R^{50a})₂, —N(R^{50a})—C(O)—R^{50b}, —N(R^{50a})—N(R^{50a})₂, —NO₂, —C(O)—H, —C(O)—R^{50b}, —C(O)₂R^{50a}, —C(O)—N(R^{50a})₂, —O—C(O)—N(R^{50a})₂, —N(R^{50a})—C(O)₂R^{50a}, —N(R^{50a})—C(O)—N(R^{50a})₂, —O—C(O)—R^{50b}, —S(O)—R^{50b}, —S(O)₂—R^{50b}, —S(O)₂—N(R^{50a})₂, —N(R^{50a})—S(O)₂—R^{50b}, —Si(R^{50b)}₃, —C(R^{50a})═N(R^{50a}), and —C(R^{50a})═N(OR^{50a}), wherein said C₁₋₆alkyl, C₂₋₆alkenyl, C₂₋₆alkynyl, carbocyclyl, and heterocyclyl in each occurrence are optionally and independently substituted on carbon with one or more R^{e}, and wherein any —NH— moiety of said heterocyclyl is optionally substituted with R^{e}*;
R⁵⁰* in each occurrence is independently selected from C₁₋₆alkyl, carbocyclyl, heterocyclyl, —C(O)—H, —C(O)—R^{50b}, —C(O)₂R^{50c}, —C(O)—N(R^{50a})₂, —S(O)—R^{50b}, —S(O)₂—R^{50b}, —S(O)₂—N(R^{50a})₂, —C(R^{50a})═N—R^{50a}, and —C(R^{50a})═N—OR^{50a}, wherein said C₁₋₆alkyl, carbocyclyl, and heterocyclyl in each occurrence are optionally and independently substituted on carbon with one or more R^{e}, and wherein any —NH— moiety of said heterocyclyl is optionally substituted with R^{e}*;
R^{50a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{e}, and wherein any —NH— moiety of said heterocyclyl is optionally substituted with R^{e}*;
R^{50b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{e}, and wherein any —NH— moiety of said heterocyclyl is optionally substituted with R^{e}*;
R^{50c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{e}, and wherein any —NH— moiety of said heterocyclyl is optionally substituted with R^{e}*;
R⁶⁰ in each occurrence is independently selected from halo, —CN, C₁₋₆alkyl, C₂₋₆alkenyl, C₂₋₆alkynyl, carbocyclyl, heterocyclyl, —OR^{60a}, —SR^{60a}, —N(R^{60a})₂, —N(R^{60a})—C(O)—R^{60b}, —N(R^{60a})—N(R^{60a})₂, —NO₂, —C(O)—H, —C(O)—R^{60b}, —C(O)₂R^{60a}, —C(O)—N(R^{60a})₂, —O—C(O)—N(R^{60a})₂, —N(R^{60a})—C(O)₂R^{60a}, —N(R^{60a})—C(O)—N(R^{60a})₂, —O—C(O)—R^{60b}), —S(O)—R^{60b}, —S(O)₂—R^{60b}), —S(O)₂—N(R^{60a})₂, —N(R^{60a})—S(O)₂—R^{60b}, —C(R^{60a})═N—R^{60a}, and —C(R^{60a})═N—OR^{60a}, wherein said C₂₋₆alkenyl, C₂₋₆alkynyl, carbocyclyl, and heterocyclyl in each occurrence are optionally and independently substituted on carbon with one or more R^{f}, and wherein any —NH— moiety of said heterocyclyl is optionally substituted with R^{f}*;
R⁶⁰* in each occurrence is independently selected from C₁₋₆alkyl, carbocyclyl, heterocyclyl, —C(O)—H, —C(O)—R^{60b}, —C(O)₂R^{60c}, —C(O)—N(R^{60a})₂, —S(O)—R^{60b}, —S(O)₂—R^{60b}, —S(O)₂—N(R^{60a})₂, —C(R^{60a})═N—R^{60a}, and —C(R^{60a})═N—OR^{60a}, wherein said C₁₋₆alkyl, carbocyclyl, and heterocyclyl in each occurrence are optionally and independently substituted on carbon with one or more R^{f}, and wherein any —NH— moiety of said heterocyclyl is optionally substituted with R^{f}*;
R^{60a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein any —NH— moiety of said heterocyclyl is optionally substituted with R^{f}*;
R^{60b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{f}, and wherein any —NH— moiety of said heterocyclyl is optionally substituted with R^{f}*;
R^{60c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{f}, and wherein any —NH— moiety of said heterocyclyl is optionally substituted with R^{f}*;
R⁷⁰ in each occurrence is independently selected from halo, —CN, C₁₋₆alkyl, C₂₋₆alkenyl, C₂₋₆alkynyl, carbocyclyl, heterocyclyl, —OR^{70a}, —SR^{70a}, —N(R^{70a})₂, —N(R^{70a})—C(O)—R^{70b}, —N(R^{70a})—N(R^{70a})₂, —NO₂, —C(O)—H, —C(O)—R^{70b}, —C(O)₂R^{70a}, —C(O)—N(R^{70a})₂, —O—C(O)—N(R^{70a})₂, —N(R^{70a})—C(O)₂R^{70a}, —N(R^{70a})—C(O)—N(R^{70a})₂, —O—C(O)—R^{70b}, —S(O)—R^{70b}, —S(O)₂—R^{70b}, —S(O)₂—N(R^{70a})₂, —N(R^{70a})—S(O)₂—R^{70b}, —C(R^{70a})═N—R^{70a}, and —C(R^{70a})═N—OR^{70a}, wherein said C₁₋₆alkyl, C₂₋₆alkenyl, C₂₋₆alkynyl, carbocyclyl, and heterocyclyl in each occurrence are optionally and independently substituted on carbon with one or more R^{g}, and wherein any —NH— moiety of said heterocyclyl is optionally substituted with R^{g}*;
R⁷⁰* in each occurrence is independently selected from C₁₋₆alkyl, carbocyclyl, heterocyclyl, —C(O)—H, —C(O)—R^{70b}, —C(O)₂R^{70c}, —C(O)—N(R^{70a})₂, —S(O)—R^{70b}, —S(O)₂—R^{70b}, —(O)₂—N(R^{70a})₂, —C(R^{70a})═N—R^{70a}, and —C(R^{70a})═N—OR^{70a}, wherein said C₁₋₆alkyl, carbocyclyl, and heterocyclyl in each occurrence are optionally and independently substituted on carbon with one or more R^{g}, and wherein any —NH— moiety of said heterocyclyl is optionally substituted with R^{g}*;
R^{70a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein any —NH— moiety of said heterocyclyl is optionally substituted with R^{g}*;
R^{70b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{g}, and wherein any —NH— moiety of said heterocyclyl is optionally substituted with R^{g}*;
R^{70c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein any —NH— moiety of said heterocyclyl is optionally substituted with R^{g}*;
R^{a}, R^{b}, R^{c}, R^{d}, R^{e}, R^{f}, and R^{g} in each occurrence are independently selected from halo, —CN, C₁₋₆alkyl, C₂₋₆alkenyl, C₂₋₆alkynyl, carbocyclyl, heterocyclyl, —OR^{m}, —SR^{m}, —N(R^{m})₂, —N(R^{m})—C(O)—Rⁿ, —N(R^{m})—N(R^{m})₂, —NO₂, —C(O)—H, —C(O)—Rⁿ, —C(O)₂R^{m}, —C(O)—N(R^{m})₂, —O—C(O)—N(R^{m})₂, —N(R^{m})—C(O)₂R^{m}, —N(R^{m})—C(O)—N(R^{m})₂, —O—C(O)—Rⁿ, —S(O)—Rⁿ, —S(O)₂—Rⁿ, —S(O)₂—N(R^{m})₂, —N(R^{m})—S(O)₂—Rⁿ, —C(R^{m})═N—R^{m}, and —C(R^{m})═N—OR^{m};
R^{a}*, R^{b}*, R^{c}*, R^{d}*, R^{e}*, R^{f}*, and R^{g} in each occurrence are independently selected from C₁₋₆alkyl, carbocyclyl, heterocyclyl, —C(O)—H, —C(O)—Rⁿ, —C(O)₂R^{o}, —C(O)—N(R^{m})₂, —S(O)—Rⁿ, —S(O)₂—Rⁿ, —S(O)₂—N(R^{m})₂, —C(R^{m})═N—R^{m}, and —C(R^{m})═N—OR^{m};
R^{m} in each occurrence is independently selected from H, C₁₋₆alkyl, carbocyclyl, and heterocyclyl;
Rⁿ in each occurrence is independently selected from C₁₋₆alkyl, C₂₋₆alkenyl, C₂₋₆alkynyl, carbocyclyl, and heterocyclyl;
R^{o} in each occurrence is independently selected from C₁₋₆alkyl, carbocyclyl, and heterocyclyl;
W in each occurrence is independently selected from —O—, —S—, —N(R^{3a})—, —N(R^{3a})C(O)—, —C(O)—, —C(O)₂—, —C(O)—N(R^{3a})—, —O—C(O)—N(R^{3a})—, —N(R^{3a})—C(O)₂—, —S(O)—, —S(O)₂—, —S(O)₂—, and —N(R^{3a})—S(O)₂—; and
X in each occurrence is independently selected from C₁₋₆alkylene, C₂₋₆alkenylene, and C₂₋₆alkynylene, wherein said C₁₋₆alkylene, C₂₋₆alkenylene, and C₂₋₆alkynylene in each occurrence are optionally and independently substituted one or more R⁴⁰.

In this specification the prefix C_{x-y} as used in terms such as C_{x-y}alkyl and the like (where x and y are integers) indicates the numerical range of carbon atoms that are present in the group; for example, C₁₋₄alkyl includes C₁alkyl (methyl), C₂alkyl (ethyl), C₃alkyl (propyl and isopropyl) and C₄alkyl (butyl, 1-methylpropyl, 2-methylpropyl, and t-butyl).

Unless specifically stated, the bonding atom of a group may be any suitable any suitable atom of that group; for example, propyl includes prop-1-yl and prop-2-yl.

Where a particular R group (e.g. R^{1a}, R¹⁰, etc.) is present in a compound of Formula (I) more than once, it is intended that each selection for that R group is independent at each occurrence of any selection at any other occurrence. For example, the —N(R)₂ group is intended to encompass: 1) those —N(R)₂ groups in which both R substituents are the same, such as those in which both R substituents are, for example, C₁₋₈alkyl; and 2) those —N(R)₂ groups in which each R substituent is different, such as those in which one R substituent is, for example, H, and the other R substituent is, for example, carbocyclyl.

With regard to divalent linker W, it is intended that for each definition provided therefor, the left-most portion of that definition's moiety is the point of attachment. For example, a compound of Formula (I) in which:

### R³ is —W—R⁶;

### R⁴ is H;

### W is —N(R^{3a})—S(O)₂—; and

n is 1,
would have the following structure:

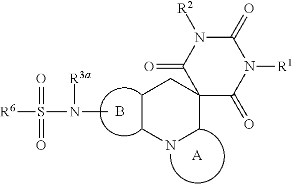

Alkyl—As used herein the term “alkyl” refers to both straight and branched chain saturated hydrocarbon radicals having the specified number of carbon atoms. References to individual alkyl groups such as “propyl” are specific for the straight chain version only and references to individual branched chain alkyl groups such as ‘isopropyl’ are specific for the branched chain version only.

Alkylene—As used herein the term “alkylene” refers to both straight and branched chain saturated hydrocarbon diradicals having the specified number of carbon atoms. For example, “C₁₋₆alkylene” includes, but is not limited to, groups such as C₁₋₃alkylene, methylene, ethylene, propylene, isopropylene, butylene, pentylene, and hexylene.

Alkenyl—As used herein, the term “alkenyl” refers to both straight and branched chain hydrocarbon radicals having the specified number of carbon atoms and containing at least one carbon-carbon double bond. For example, “C₂₋₆alkenyl” includes, but is not limited to, groups such as C₂₋₅alkenyl, C₂₋₄alkenyl, ethenyl, 2-propenyl, 2-methyl-2-propenyl, 3-butenyl, 4-pentenyl, 5-hexenyl, 2-heptenyl, and 2-methyl-1-heptenyl.

Alkenylene—As used herein, the term “alkenylene” refers to both straight and branched chain hydrocarbon radicals having the specified number of carbon atoms and containing at least one carbon-carbon double bond. In one aspect, “alkenylene” may be ethene-1,2-diyl.

Alkynyl—As used herein, the term “alkynyl” refers to both straight and branched chain hydrocarbon radicals having the specified number of carbon atoms and containing at least one carbon-carbon triple bond. For example, “C₂₋₈alkynyl” includes, but is not limited to, groups such as C₂₋₆alkynyl, C₂₋₄alkynyl, ethynyl, 2-propynyl, 2-methyl-2-propynyl, 3-butynyl, 4-pentynyl, 5-hexynyl, 2-heptynyl, and 4-methyl-5-heptynyl.

Alkynylene—As used herein, the term “alkynylene” refers to both straight and branched chain hydrocarbon radicals having the specified number of carbon atoms and containing at least one carbon-carbon triple bond. In one aspect, “alkynylene” may be ethyne-1,2-diyl.

Halo—As used herein, the term “halo” is intended to include fluoro, chloro, bromo and iodo. In one aspect, the “halo” may refer fluoro, chloro, and bromo. In another aspect, “halo” may refer to fluoro and chloro. In still another aspect, “halo” may refer to fluoro. In yet another aspect, “halo” may refer to chloro.

Carbocyclyl—As used herein, the term “carbocyclyl” refers to a saturated, partially saturated, or unsaturated, mono or bicyclic carbon ring that contains 3-12 atoms, wherein one or more —CH₂-groups may optionally be replaced by a corresponding number of —C(O)— groups. In one aspect, the term “carbocyclyl” may refer to a monocyclic ring containing 5 or 6 atoms or a bicyclic ring containing 9 or 10 atoms. Illustrative examples of “carbocyclyl” include, but are not limited to, adamantyl, cyclopropyl, cyclobutyl, cyclopentyl, cyclopentenyl, cyclohexyl, cyclohexenyl, 1-oxocyclopentyl, phenyl, naphthyl, tetralinyl, indanyl or 1-oxoindanyl. In one aspect, “carbocyclyl” may be phenyl. In another aspect, “carbocyclyl” may be selected from cyclopropyl, cyclobutyl, cyclopentyl, cyclopentenyl, and cyclohexyl.

3- to 6-Membered Carbocyclyl—In one aspect, “carbocyclyl” may be “3- to 6-membered carbocyclyl.” The term “3- to 6-membered carbocyclyl” refers to a saturated or partially saturated monocyclic carbon ring containing 3 to 6 ring atoms, of which one or more —CH₂-groups may be optionally replaced with a corresponding number of —C(O)— groups. Illustrative examples of “3- to 6-membered carbocyclyl” include cyclopropyl, cyclobutyl, cyclopentyl, oxocyclopentyl, cyclopentenyl, cyclohexyl, and phenyl.

Heterocyclyl—As used herein, the term “heterocyclyl” refers to a saturated, partially saturated or unsaturated, mono or bicyclic ring containing 4 to 12 atoms of which at least one atom is selected from nitrogen, sulfur or oxygen, which may, unless otherwise specified, be carbon or nitrogen linked, wherein a —CH₂— group can optionally be replaced by a —C(O)—. Ring sulfur atoms may be optionally oxidized to form S-oxides. Ring nitrogen atoms may be optionally oxidized to form N-oxides. Illustrative examples of the term “heterocyclyl” include, but are not limited to, benzimidazolyl, 1,3-benzodioxolyl, benzofuranyl, 1-benzothiophenyl, 1,3-benzothiazolyl, 1,3-benzoxazolyl, dioxidotetrahydrothiophenyl, 3,5-dioxopiperidinyl, imidazolyl, indolyl, isoquinolone, isothiazolyl, isoxazolyl, morpholinyl, 1,2,4-oxadiazolyl, oxoimidazolidinyl, 2-oxopyrrolidinyl, 2-oxotetrahydrofuranyl, 2-oxo-1,3-thiazolidinyl, piperazinyl, piperidylpiperidinyl, pyranyl, pyrazolyl, pyridinyl, pyrrolyl, pyrrolidinyl, pyrrolinyl, pyrimidyl, pyrazinyl, pyrazolyl, pyridazinyl, 4-pyridone, quinolyl, tetrazolyl, tetrahydrofuranyl, tetrahydropyranyl, thiazolyl, 1,3,4-thiadiazolyl, thiazolidinyl, thienyl, thiomorpholino, 4H-1,2,4-triazolyl, pyridine-N-oxide and quinoline-N-oxide. In one aspect of the invention the term “heterocyclyl” may refer to a saturated, partially saturated, or unsaturated, monocyclic ring containing 5 or 6 atoms of which at least one atom is chosen from nitrogen, sulfur or oxygen, and may, unless otherwise specified, be carbon or nitrogen linked, and a ring nitrogen atom may be optionally oxidized to form an N-oxide.

9- or 10-Membered Bicyclic Heteroaryl—In one aspect, “heterocyclyl” may be “9- or 10-membered bicyclic heteroaryl.” The term “9- or 10-membered bicyclic heteroaryl” is intended to refer to bicyclic aromatic heterocyclic ring containing 9 or 10 ring atoms, of which at least one ring atom is selected from nitrogen, sulfur, and oxygen, and which may, unless otherwise specified, be carbon or nitrogen linked. Ring nitrogen atoms may be optionally oxidized to form an N-oxide. Ring sulfur atoms may be optionally oxidized to form S-oxides. Illustrative examples of “9- or 10-membered bicyclic ring” include benzimidazolyl, quinolinyl, benzofuranyl, 1-benzothiophenyl, 1,3-benzothiazolyl, 1,3-benzoxazolyl, indolyl, and isoquinolinyl.

5- or 6-Membered Heterocyclyl—In one aspect, “heterocyclyl” may be “5- or 6-membered heterocyclyl,” which refers to a saturated, partially saturated, or unsaturated, monocyclic ring containing 5 or 6 ring atoms, of which at least one ring atom is selected from nitrogen, sulfur, and oxygen, and of which a —CH₂— group may be optionally replaced by a —C(O)— group. Unless otherwise specified, “5- or 6-membered heterocyclyl” groups may be carbon or nitrogen linked. Ring nitrogen atoms may be optionally oxidized to form an N-oxide. Ring sulfur atoms may be optionally oxidized to form S-oxides. Illustrative examples of “5- or 6-membered heterocyclyl” include dioxidotetrahydrothiophenyl, 2,4-dioxoimidazolidinyl, 3,5-dioxopiperidinyl, furanyl, imidazolyl, isothiazolyl, isoxazolyl, morpholinyl, oxazolyl, oxoimidazolidinyl, 2-oxopyrrolidinyl, 2-oxotetrahydrofuranyl, oxo-1,3-thiazolidinyl, piperazinyl, piperidinyl, 2H-pyranyl, pyrazolyl, pyridinyl, pyrrolyl, pyrrolidinyl, pyrrolidinyl, pyrimidinyl, pyrazinyl, pyrazolyl, pyridazinyl, 4-pyridonyl, tetrazolyl, tetrahydrofuranyl, tetrahydropyranyl, thiazolyl, 1,3,4-thiadiazolyl, 1,34-thiazolidinyl, thiomorpholinyl, thiophenyl, 4H-1,2,4-triazolyl, and pyridine-N-oxidyl.

5 or 6-Membered Non-Aromatic Heterocyclyl—In one aspect, “heterocyclyl” and “5- or 6-membered heterocyclyl” may be “5 or 6-membered non-aromatic heterocyclyl.” The term “5- or 6-membered non-aromatic heterocyclyl” is intended to refer to a saturated or partially saturated, monocyclic, non-aromatic heterocyclyl ring containing 5 or 6 ring atoms, of which at least one ring atom is selected from nitrogen, sulfur, and oxygen, and which may, unless otherwise specified, be carbon or nitrogen linked, and of which a —CH₂— group can optionally be replaced by a —C(O)—. Ring sulfur atoms may be optionally oxidized to form S-oxides. Ring nitrogen atoms may be optionally oxidized to form N-oxides. Illustrative examples of “5 or 6-membered non-aromatic heterocyclyl” include dioxidotetrahydrothiophenyl, 2,4-dioxoimidazolidinyl, 3,5-dioxopiperidinyl, morpholinyl, oxoimidazolidinyl, 2-oxopyrrolidinyl, 2-oxotetrahydrofuranyl, oxo-1,3-thiazolidinyl, piperazinyl, piperidinyl, 2H-pyranyl, pyrrolidinyl, tetrahydrofuranyl, tetrahydropyranyl, thiomorpholinyl, and thiazolidinyl.

5- or 6-Membered Heteroaryl—In one aspect, “heterocyclyl” and “5- or 6-membered heterocyclyl” may be “5- or 6-membered heteroaryl.” The term “5- or 6-membered heteroaryl” is intended to refer to a monocyclic, aromatic heterocyclyl ring containing 5 or 6 ring atoms, of which at least one ring atom is selected from nitrogen, sulfur, and oxygen. Unless otherwise specified, “6-membered heteroaryl” groups may be carbon or nitrogen linked. Ring nitrogen atoms may be optionally oxidized to form an N-oxide. Ring sulfur atoms may be optionally oxidized to form S-oxides. Illustrative examples of “5- or 6-membered heteroaryl” include furanyl, imidazolyl, isothiazolyl, isoxazole, oxazolyl, pyrazinyl, pyrazolyl, pyridazinyl, pyrimidinyl, pyridinyl, pyrrolyl, tetrazolyl, 1,3,4-thiadiazolyl, thiazolyl, thiophenyl, 4H-1,2,4-triazolyl.

6-Membered Heteroaryl—In one aspect, “heterocyclyl,” 5- or 6-membered heterocyclyl,” and “5- or 6-membered heteroaryl” may be “6-membered heteroaryl.” The term “6-membered heteroaryl” is intended to refer to a monocyclic, aromatic heterocyclyl ring containing 6 ring atoms. Ring nitrogen atoms may be optionally oxidized to form an N-oxide. Illustrative examples of “6-membered heteroaryl” include pyrazinyl, pyridazinyl, pyrimidinyl, and pyridinyl.

5- to 7-Membered Non-Aromatic Heterocyclic Ring—For the purposes of Ring A, the term “5-to 7-membered non-aromatic heterocyclic ring” is intended to refer to a saturated or partially saturated, monocyclic, non-aromatic heterocyclic ring containing - to 7 ring atoms, which may contain, in addition to the bridgehead nitrogen shown in Formula (I), a member selected from —O—, —NH—, and —S—, and of which a —CH₂— group can optionally be replaced by a —C(O)—. Ring sulfur atoms may be optionally oxidized to form S-oxides. Ring nitrogen atoms may be optionally oxidized to form N-oxides. Illustrative examples of “5- to 7-membered non-aromatic heterocyclic ring” include 3,5-dioxopiperidine, morpholine, 2-oxopyrrolidine, 2-oxotetrahydrofuranyl, oxo-1,3-thiazolidine, piperazine, piperidine, 2H-pyrane, pyrrolidine, thiomorpholine, and thiazolidine. In one aspect, “5- to 7-membered non-aromatic heterocyclic ring” is morpholine.

For the purposes of Ring A, the term “morpholine” is intended to denote the following structure:

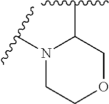

which is optionally substituted on carbon by R⁷ as indicated herein.

For the purposes of Ring A, the term “piperazine” is intended to denote the following structure:

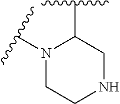

which is optionally substituted on carbon by R⁷, and optionally substituted on nitrogen by R⁷*, each as indicated herein.

For the purposes of Ring A, the term “piperidine” is intended to denote the following structure:

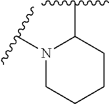

which is optionally substituted on carbon by R⁷, as indicated herein.

For the purposes of Ring A, the term “6-methylpiperazin-2-one” is intended to denote the following structure:

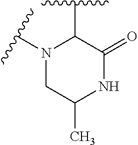

For the purposes of Ring B, the term “pyridine” is intended to denote any of the following structures:

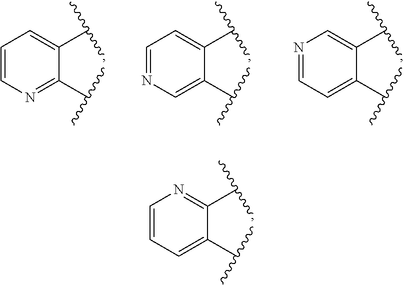

wherein the structures may be optionally substituted on carbon by R³ and R⁴ as indicated herein.

Optionally substituted—As used herein, the phrase “optionally substituted” indicates that substitution is optional and therefore it is possible for the designated group to be either substituted or unsubstituted. In the event a substitution is desired, the appropriate number of hydrogens on the designated group may be replaced with a selection from the indicated substituents, provided that the normal valency of the atoms on a particular substituent is not exceeded, and that the substitution results in a stable compound.

In one aspect, when a particular group is designated as being optionally substituted with one or more substituents, the particular group may be unsubstituted. In another aspect, the particular group may bear one substituent. In another aspect, the particular substituent may bear two substituents. In still another aspect, the particular group may bear three substituents. In yet another aspect, the particular group may bear four substituents. In a further aspect, the particular group may bear one or two substituents. In still a further aspect, the particular group may be unsubstituted, or may bear one or two substituents.

Pharmaceutically Acceptable—As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Effective Amount—As used herein, the phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician.

Leaving Group—As used herein, the phrase “leaving group” is intended to refer to groups readily displaceable by a nucleophile such as an amine nucleophile, and alcohol nucleophile, or a thiol nucleophile. Examples of suitable leaving groups include halo, such as chloro and bromo, and sulfonyloxy group, such as methanesulfonyloxy and toluene-4-sulfonyloxy.

Protecting Group—As used herein, the term “protecting group” is intended to refer to those groups used to prevent selected reactive groups (such as carboxy, amino, hydroxy, and mercapto groups) from undergoing undesired reactions.

Illustrative examples of suitable protecting groups for a hydroxy group include, but are not limited to, an acyl group; alkanoyl groups such as acetyl; aroyl groups, such as benzoyl; silyl groups, such as trimethylsilyl; and arylmethyl groups, such as benzyl. The deprotection conditions for the above hydroxy protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

Illustrative examples of suitable protecting groups for an amino group include, but are not limited to, acyl groups; alkanoyl groups such as acetyl; alkoxycarbonyl groups, such as methoxycarbonyl, ethoxycarbonyl, and t-butoxycarbonyl; arylmethoxycarbonyl groups, such as benzyloxycarbonyl; and aroyl groups, such benzoyl. The deprotection conditions for the above amino protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric, phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid, for example boron trichloride). A suitable alternative protecting group for a primary amino group is, for example, a phthaloyl group, which may be removed by treatment with an alkylamine, for example dimethylaminopropylamine or 2-hydroxyethylamine, or with hydrazine. Another suitable protecting group for an amine is, for example, a cyclic ether such as tetrahydrofuran, which may be removed by treatment with a suitable acid such as trifluoroacetic acid.

The protecting groups may be removed at any convenient stage in the synthesis using conventional techniques well known in the chemical art, or they may be removed during a later reaction step or during work-up.

Compounds of Formula (I) may form stable pharmaceutically acceptable acid or base salts, and in such cases administration of a compound as a salt may be appropriate. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethyl-sulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Examples of base salts include ammonium salts; alkali metal salts such as sodium, lithium and potassium salts; alkaline earth metal salts such as aluminum, calcium and magnesium salts; salts with organic bases such as dicyclohexylamine salts and N-methyl-D-glucamine; and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates such as dimethyl, diethyl, dibutyl; diamyl sulfates; long chain halides such as decyl, lauryl, myristyl and stearyl halides; arylalkyl halides such as benzyl bromide and others. Non-toxic physiologically-acceptable salts are preferred, although other salts may be useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

Some compounds of Formula (I) may have chiral centres and/or geometric isomeric centres (E- and Z-isomers), and it is to be understood that the invention encompasses all such optical, diastereoisomers and geometric isomers. The invention further relates to any and all tautomeric forms of the compounds of Formula (I).

It is also to be understood that certain compounds of Formula (I) can exist in solvated as well as unsolvated forms such as, for example, hydrated forms. It is to be understood that the invention encompasses all such solvated forms.

Additional embodiments of the invention are as follows. These additional embodiments relate to compounds of Formula (I) and pharmaceutically acceptable salts thereof. Such specific substituents may be used, where appropriate, with any of the definitions, claims or embodiments defined hereinbefore or hereinafter.

### Ring A

In one aspect, Ring A is a 6-membered non-aromatic heterocyclic ring, wherein
1) said 6-membered non-aromatic heterocyclic ring optionally contains, in addition to the nitrogen, a member selected from —O— and —NH—;
2) said 6-membered non-aromatic heterocyclic ring is optionally substituted on carbon with one or more R⁷; and
3) any —NH— moiety of said 6-membered non-aromatic heterocyclic ring is optionally and independently substituted with R⁷*;
   R⁷ is C₁₋₆alkyl;
   R⁷* in each occurrence is independently selected from H and —C(O)₂R^{7c}; and
   R^{7c} is C₁₋₆alkyl.

In another aspect, Ring A is a 6-membered non-aromatic heterocyclic ring, wherein
1) said 6-membered non-aromatic heterocyclic ring optionally contains, in addition to the nitrogen, a member selected from —O— and —NH—; and
2) said 6-membered non-aromatic heterocyclic ring is optionally substituted on carbon with one or more R⁷; and
   R⁷ is C₁₋₆alkyl.

In still another aspect, Ring A is selected from morpholine, piperazine, and piperidine, wherein
1) said morpholine, piperazine, and piperidine are optionally substituted on carbon with one or more R⁷; and
2) the —NH— moiety of said piperazine is optionally substituted with R⁷*;
   R⁷ is C₁₋₆alkyl; and
   R⁷* in each occurrence is independently selected from H and —C(O)₂R^{7c}; and
   R^{7c} is C₁₋₆alkyl.

In yet another aspect, Ring A is selected from morpholine, piperazine, and piperidine, wherein said morpholine, piperazine, and piperidine are optionally substituted on carbon with one or more R⁷, and wherein a —CH₂— group of said morpholine, piperazine, and piperidine can optionally be replaced by a —C(O)—; and

R⁷ is C₁₋₆alkyl.

In a further aspect, Ring A is selected from morpholine, piperazine, and piperidine, wherein said morpholine, piperazine, and piperidine are optionally substituted on carbon with one or more R⁷; and

R⁷ is C₁₋₆alkyl.

In still a further aspect, Ring A is selected from morpholine, piperazine, and piperidine, wherein:
1) said morpholine, piperazine, and piperidine are optionally substituted on carbon with one or more methyl; and
2) the —NH— moiety of said piperazine is optionally substituted with t-butoxycarbonyl.

In yet a further aspect, Ring A is selected from 1-t-butoxycarbonylpiperazine, 2,6-dimethylmorpholine, 3,5-dimethylpiperidine piperidine, and piperazine.

In one aspect, Ring A is selected from 2,6-dimethylmorpholine, 3,5-dimethylpiperidine, 6-methylpiperazin-2-one, and piperidine.

In another aspect, Ring A is 2,6-dimethylmorpholine.

### Ring B

In one aspect, Ring B is a 6-membered aromatic heterocyclic ring.

In another aspect, Ring B is pyridine.

In still another aspect, Ring B is selected from:

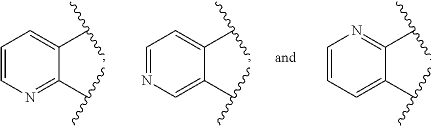

In yet another aspect, Ring B is:

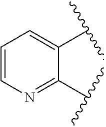

In a further aspect, Ring B is:

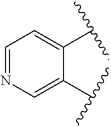

In still a further aspect, Ring B is:

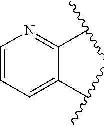

### R¹

In one aspect, R¹ is selected from H and C₁₋₆alkyl.

In another aspect, R¹ is H.

In still another aspect, R¹ is C₁₋₆alkyl.

In yet another aspect, R¹ is selected from H and C₁₋₆alkyl.

In a further aspect, R¹ is selected from H and methyl.

In still a further aspect, R¹ is methyl.

### R²

In one aspect, R² is selected from H and C₁₋₆alkyl.

In another aspect, R² is H.

In still another aspect, R² is C₁₋₆alkyl.

In yet another aspect, R² is selected from H and C₁₋₆alkyl.

In a further aspect, R² is selected from H and methyl.

In still a further aspect, R¹ is methyl.

### R¹ and R²

In one aspect, R¹ and R² are H.

### R³

In one aspect, R³ in each occurrence is independently selected from —X—R⁵ and —C(NH₂)═N—OH; R⁵ in each occurrence is independently selected from phenyl and 5- or 6-membered heteroaryl, wherein said phenyl and 5- or 6-membered heteroaryl in each occurrence are optionally and independently substituted with one or more R⁵⁰;

### R⁵⁰ is —OR^{50a};

R^{50a} is C₁₋₆alkyl; and
X is ethyne-1,2-diyl.

In another aspect, R³ is —X—R⁵;

R⁵ in each occurrence is independently selected from phenyl and 5- or 6-membered heteroaryl, wherein said phenyl and 5- or 6-membered heteroaryl in each occurrence are optionally and independently substituted with one or more R⁵⁰;

### R⁵⁰ is —OR^{50a};

R^{50a} is C₁₋₆alkyl; and
X is ethyne-1,2-diyl.

In still another aspect, R³ in each occurrence is independently selected from —C(NH₂)═N—OH, 4-methoxyphenylethynyl, and pyrazin-2-ylethynyl.

In yet another aspect, R³ in each occurrence is independently selected from 4-methoxyphenylethynyl and pyrazin-2-ylethynyl.

### R⁴

In one aspect, R⁴ in each occurrence is independently selected from H and halo.

In another aspect, R⁴ in each occurrence is independently selected from H, fluoro, and bromo.

In still another aspect, R⁴ in each occurrence is independently selected from H, fluoro, chloro, bromo, and iodo.

In yet another aspect, R⁴ is fluoro.

In a further aspect, R⁴ is bromo.

In still a further aspect, R⁴ is H.

In yet a further aspect, R⁴ in each occurrence is independently selected from H, —CN, halo, phenyl, 5- or 6-membered heteroaryl, and 9- or 10-membered bicyclic heteroaryl, wherein said phenyl, 5- or 6-membered heteroaryl, and 9- or 10-membered bicyclic heteroaryl in each occurrence are optionally substituted with one or more R⁴⁰, and wherein any —NH— moiety of said 5- or 6-membered heteroaryl is optionally substituted with R⁴⁰*;

R⁴⁰ in each occurrence is independently selected from halo, C₁₋₆alkyl, phenyl, 5- or 6-membered heterocyclyl, —OR^{40a}, and —N(R^{40a})₂;
R⁴⁰* is C₁₋₆alkyl; and
R^{40a} in each occurrence is independently selected from H and C₁₋₆alkyl.

In one aspect, R⁴ in each occurrence is independently selected from H, —CN, halo, phenyl, and 5- or 6-membered heteroaryl, wherein said phenyl and 5- or 6-membered heteroaryl in each occurrence are optionally substituted with one or more R⁴⁰, and wherein any —NH— moiety of said 5- or 6-membered heteroaryl is optionally substituted with R⁴⁰*;

R⁴⁰ in each occurrence is independently selected from halo, C₁₋₆alkyl, phenyl, 5- or 6-membered heterocyclyl, —OR^{40a}, and —N(R^{40a})₂;
R⁴⁰* is C₁₋₆alkyl; and
R^{40a} in each occurrence is independently selected from H and C₁₋₆alkyl.

In another aspect, R⁴ in each occurrence is independently selected from H, —CN, halo, and 5- or 6-membered heteroaryl, wherein said 5- or 6-membered heteroaryl in each occurrence is optionally substituted with one or more R⁴⁰, and wherein any —NH— moiety of said 5- or 6-membered heteroaryl is optionally substituted with R⁴*;

R⁴⁰ in each occurrence is independently selected from halo, C₁₋₆alkyl, phenyl, 5- or 6-membered heterocyclyl, —OR^{40a}, and —N(R^{40a})₂;
R⁴⁰* is C₁₋₆alkyl; and
R^{40a} in each occurrence is independently selected from H and C₁₋₆alkyl.

In still another aspect, R⁴ in each occurrence is independently selected from H, —CN, halo, and 9- or 10-membered bicyclic heteroaryl.

In yet another aspect, R⁴ in each occurrence is independently selected from H, benzimidazolyl, benzofuranyl, 1,3-benzothiazolyl, 1-benzothiophenyl, bromo, chloro, fluoro, furanyl, imidazolyl, iodo, isoxazolyl, 1,2,4-oxadiazolyl, oxazolyl, phenyl, pyrazinyl, pyrazolyl, pyridazinyl, pyridinyl, pyrimidinyl, quinolinyl, tetrazolyl, 1,3,4-thiadiazolyl, thiazolyl, and thiophenyl, wherein said benzimidazolyl, benzofuranyl, 1,3-benzothiazolyl, 1-benzothiophenyl, furanyl, imidazolyl, isoxazolyl, 1,2,4-oxadiazolyl, oxazolyl, phenyl, pyrazinyl, pyrazolyl, pyridazinyl, pyridinyl, pyrimidinyl, quinolinyl, tetrazolyl, 1,3,4-thiadiazolyl, thiazolyl, and thiophenyl in each occurrence are optionally substituted with one or more R⁴⁰, and wherein any —NH— moiety of said imidazolyl, pyrazolyl, and tetrazolyl is optionally substituted with R⁴⁰*;

R⁴⁰ in each occurrence is independently selected from chloro, fluoro, dimethylamino, methoxy, methyl, morpholinyl, phenyl, pyrazolyl, and tetrazolyl; and
R⁴⁰* is methyl.

In a further aspect, R⁴ in each occurrence is independently selected from H, bromo, chloro, fluoro, furanyl, imidazolyl, iodo, isoxazolyl, 1,2,4-oxadiazolyl, oxazolyl, phenyl, pyrazinyl, pyrazolyl, pyridazinyl, pyridinyl, pyrimidinyl, tetrazolyl, 1,3,4-thiadiazolyl, thiazolyl, and thiophenyl, wherein said furanyl, imidazolyl, isoxazolyl, 1,2,4-oxadiazolyl, oxazolyl, phenyl, pyrazinyl, pyrazolyl, pyridazinyl, pyridinyl, pyrimidinyl, tetrazolyl, 1,3,4-thiadiazolyl, thiazolyl, and thiophenyl in each occurrence are optionally substituted with one or more R⁴⁰, and wherein any —NH— moiety of said imidazolyl, pyrazolyl, and tetrazolyl is optionally substituted with R⁴⁰*;

R⁴⁰ in each occurrence is independently selected from chloro, fluoro, dimethylamino, methoxy, methyl, morpholinyl, phenyl, pyrazolyl, and tetrazolyl; and
R⁴⁰* is methyl.

In still a further aspect, R⁴ in each occurrence is independently selected from H, —CN, bromo, chloro, fluoro, iodo, 1H-benzimidazol-2-yl, 1-benzofuran-2-yl, 1,3-benzothiazol-2-yl, 1-benzothien-2-yl, 5-chloropyridin-2-yl, 2-(dimethyylamino)pyrimidin-5-yl, 3,5-dimethylisoxazol-4-yl, 2,4-dimethyl-1,3-thiazol-5-yl, 4-fluorophenyl, furan-2-yl, furan-3-yl, 1H-imidazol-2-yl, 1H-imidazol-4-yl, 2-methoxyphenyl, 3-methyoxypyrazin-2-yl, 6-methoxypyrazin-2-yl, 4-methoxypyridin-3-yl, 2-methoxy-1,3-thiaol-4-yl, 1-methyl-1H-imidazol-2-yl, 1-methyl-1H-imidazol-4-yl 1-methyl-1H-imidazol-5-yl, 2-methylphenyl, 1-methyl-1H-pyrazol-4-yl, 1-methyl-1H-pyrazol-5-yl, 1-methyl-1H-tetrazol-5-yl, 2-methyl-2H-tetrazol-5-yl, 5-methyl-1,3,4-thiadiazoly-2-yl, 3-methylthiophen-2-yl, 4-methylthiophen-3-yl, 5-methylthiophen-2-yl, 6-(morpholin-4-yl)pyridin-3-yl, 5-methyl-1,2,4-oxathiadiazol-3-yl, 1,3-oxazol-2-yl, phenyl, pyrazin-2-yl, 1H-pyrazol-4-yl, 1H-pyrazol-5-yl, 5-(1H-pyrazol-5-yl)thiophen-2-yl, pyridazin-4-yl, pyridin-2-yl, pyridin-3-yl, pyridin-4-yl, pyrimidin-2-yl, pyrimidin-5-yl, quinolin-2-yl, quinolin-8-yl, 1,3,4-thiadiazol-2-yl, 1,3-thiazol-2-yl, 1,3-thiazol-4-yl, thiazol-5-yl, thiophen-2-yl, and 5-(1H-tetrazol-5-yl)thiophen-2-yl.

### R⁴ and n

In one aspect, R⁴ in each occurrence is independently selected from H and halo; and

n is 0.

In still another aspect, R⁴ in each occurrence is independently selected from H and halo; and

n is 0,
wherein one R⁴ is halo and three R⁴ are H.

In still another aspect, R⁴ in each occurrence is independently selected from H and halo; and

n is 0,
wherein one R⁴ is halo and the remaining R⁴ are H.

In still another aspect, R⁴ in each occurrence is independently selected from H and fluoro; and

n is 0,
wherein one R⁴ is fluoro and the remaining R⁴ are H.

In yet another aspect, R⁴ in each occurrence is independently selected from H and bromo; and

n is 0,
wherein one R⁴ is bromo and the remaining R⁴ are H.
n

In one aspect, n is 0.

### Ring B, R³, R⁴, and n

In one aspect, Ring B is a 6-membered aromatic heterocyclic ring;

R⁴ in each occurrence is independently selected from H and halo; and
n is 0.

In another aspect, Ring B is pyridine;

R⁴ in each occurrence is independently selected from H and halo; and
n is 0.

In still another aspect, Ring B is a 6-membered aromatic heterocyclic ring;

R⁴ in each occurrence is independently selected from H and halo; and
n is 0,
wherein one R⁴ is halo, and the remaining R⁴ are H.

In yet another aspect, Ring B is pyridine;

R⁴ in each occurrence is independently selected from H and halo; and
n is 0,
wherein one R⁴ is halo, and the remaining R⁴ are H.

In a further aspect, Ring B is selected from

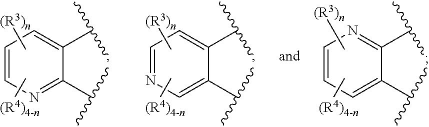

R⁴ in each occurrence is independently selected from H and halo; and
n is 0.

In still a further aspect, Ring B is selected from

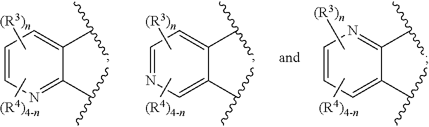

R⁴ in each occurrence is independently selected from H and halo; and
n is 0,
wherein one R⁴ is halo, and the remaining R⁴ is H.

In yet a further aspect, Ring B, R³, R⁴, and n may together form a member selected from:

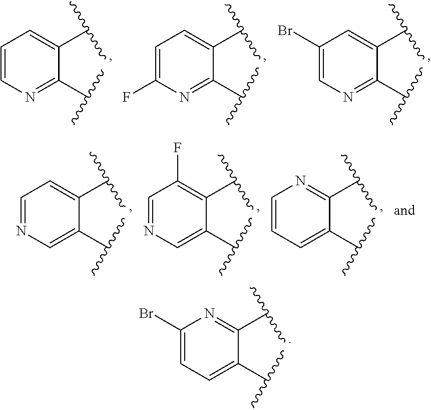

In one aspect, Ring B is pyridine;

n is 0; and
R⁴ in each occurrence is independently selected from H, —CN, halo, and 5- or 6-membered heteroaryl, wherein said 5- or 6-membered heteroaryl in each occurrence is optionally substituted with one or more R⁴⁰, and wherein any —NH— moiety of said 5- or 6-membered heteroaryl is optionally substituted with R⁴⁰*;
R⁴⁰ in each occurrence is independently selected from halo, C₁₋₆alkyl, phenyl, 5- or 6-membered heterocyclyl, —OR^{40a}, —N(R^{40a})₂;
R⁴⁰* K is C₁₋₆alkyl; and
R^{40a} in each occurrence is independently selected from H and C₁₋₆alkyl.

### Ring A, Ring B, R¹, R², R³, R⁴, and n

In one aspect, Ring A is a 5- to 7-membered non-aromatic heterocyclic ring, wherein
1) said 5- to 7-membered non-aromatic heterocyclic ring optionally contains, in addition to the nitrogen, a member selected from —O—, —NH—, —S—, —S(O)—, and —S(O)₂—;
2) said 5- to 7-membered non-aromatic heterocyclic ring is optionally substituted on carbon with one or more R⁷;
3) two R⁷ substituents on one carbon atom may together optionally form the group ═O or the group ═N(OR^{7a}); and
4) any —NH— moiety of said 5- to 7-membered heterocyclic ring is optionally substituted with R⁷*;
   Ring B is a 5- or 6-membered aromatic heterocyclic ring;
   n is 0 to 3;
   R¹ is selected from H, C₁₋₆alkyl, carbocyclyl, heterocyclyl, —C(O)—H, —C(O)—R^{1b}, —C(O)₂R^{1c}, C(O)—N(R^{1a})₂, —S(O)—R^{1b}, —S(O)₂—R^{1b}, —S(O)₂—N(R^{1a})₂, —C(R^{1a})═N—R^{1a}, and —C(R^{1a})═N—OR^{1a}, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
   R^{1a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
   R^{1b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein any —NH— moiety of said heterocyclyl is optionally substituted with R¹⁰*;
   R^{1c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
   R² is selected from H, C₁₋₆alkyl, carbocyclyl, heterocyclyl, —C(O)—H, —C(O)—R^{2b}, —C(O)₂R^{2c}, —C(O)—N(R^{2a})₂, —S(O)—R^{2b}, —S(O)₂—R^{2b}, —S(O)₂—N(R^{2a})₂, —C(R^{2a})═N—R^{2a}, and —C(R^{2a})═N—OR^{2a}, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
   R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
   R^{2b} each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein any —NH— moiety of said heterocyclyl is optionally substituted with R²⁰*;
   R^{2c} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
   R³ in each occurrence is independently selected from —X—R⁵, —W—R⁶, —C(O)—N(R^{3a})—S(O)₂—R^{3b}, —C(R^{3a})═N—R^{3y}, —C(R^{3a})═N—NR^{3a}—C(O)—R^{3b}, —C(N(R^{3a})₂)═N—R^{3y}, —C(N(R^{3a})₂)═N—OR^{3y}, —C(N(R^{3a})₂)═N—C(O)—R^{3b}, —C(N(R^{3a})₂)═N—S(O)₂—R^{3b}, —C(N(R^{3a})₂)═N—CN, —N═C(R^{3y})₂, —N(R^{3a})—S(O)₂—N(R^{3y})₂, —N(R^{3a})—N(R^{3y})₂, —N(R^{3a})—C(O)—N(R^{3y})₂, —N(R^{3a})—C(O)—N(R^{3a})—S(O)₂—R^{3b}, —N(R^{3a})—C(R^{3a})═N(R^{3y}), —N(R^{3a})—C(R^{3a})═N—OR^{3y}, —N(R^{3a})—C(R^{3a})═N—C(O)—R^{3b}, —N(R^{3a})—C(R^{3a})═N—S(O)₂R^{3b}, —N(R^{3a})—C(R^{3a})═N—CN, —N(R^{3a})—C(N(R^{3a})₂)═N—R^{3y}, —N(R^{3a})—C(N(R^{3a})₂)═N—OR^{3y}, —N(R^{3a})—C(N(R^{3a})₂)═N—C(O)—R^{3b}, —N(R^{3a})—C(N(R^{3a})₂)═N—S(O)₂—R^{3b}, —N(R^{3a})—C(N(R^{3a})₂)═N—CN, —O—C(O)—R^{3b}, and —Si(R^{3b})₃;
   R^{3a} and R^{3y} in each occurrence are independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein any —NH— moiety of said heterocyclyl is optionally substituted with R³⁰*;
   R^{3b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein any —NH— moiety of said heterocyclyl is optionally substituted with R³⁰*;
   R⁴ in each occurrence is independently selected from H, halo, —CN, C₁₋₆alkyl, C₂₋₆alkenyl, C₂₋₆alkynyl, carbocyclyl, heterocyclyl, —OR^{4d}, —SR^{4d}, —N(R^{4d})₂, —N(R^{4d})—C(O)—R^{4e}, —NO₂, —C(O)—H, —C(O)—R^{4e}, —C(O)₂R^{4d}, —C(O)—N(R^{4d})₂, —O—C(O)—N(R^{4d})₂, —N(R^{4a})—C(O)₂R^{4d}, —S(O)—R^{4e}, —S(O)₂—R^{4e}, —S(O)₂—N(R^{d})₂, —N(R^{4a})—S(O)₂—R^{4e}, and —C(R^{4a})═N—OR^{4d}, wherein said C₁₋₆alkyl, C₂₋₆alkenyl, and C₂₋₆alkynyl in each occurrence are optionally and independently substituted with one or more R^{40x}, and wherein said carbocyclyl and heterocyclyl in each occurrence are optionally and independently substituted on carbon with one or more R⁴⁰, and wherein any —NH— moiety of said heterocyclyl is optionally substituted with R⁴⁰*;
   R^{4a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁴⁰, and wherein any —NH— moiety of said heterocyclyl is optionally substituted with R⁴⁰*;
   R^{4d} in each occurrence is independently selected from H, C₁₋₆alkyl, carbocyclyl, and non-aromatic heterocyclyl, wherein said C₁₋₆alkyl, carbocyclyl, and non-aromatic heterocyclyl in each occurrence are optionally and independently substituted on carbon with one or more R⁴⁰, and wherein any —NH— moiety of said heterocyclyl is optionally substituted with R⁴⁰*;
   R^{4e} in each occurrence is selected from C₁₋₆alkyl, C₂₋₆alkenyl, C₂₋₆alkynyl, carbocyclyl, and non-aromatic heterocyclyl, wherein said C₁₋₆alkyl, C₂₋₆alkenyl, C₂₋₆alkynyl, carbocyclyl, and non-aromatic heterocyclyl in each occurrence are optionally and independently substituted on carbon with one or more R⁴⁰, and wherein any —NH— moiety of said heterocyclyl is optionally substituted with R⁴⁰*;
   R⁵ is selected from heterocyclyl and —Si(R^{5b})₃, wherein said heterocyclyl is optionally substituted on carbon with one or more R⁵⁰, and wherein any —NH— moiety of said heterocyclyl is optionally substituted with R⁵⁰*;
   R⁵ in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁴⁰, and wherein any —NH— moiety of said heterocyclyl is optionally substituted with R⁵⁰*;
   R⁶ is non-aromatic heterocyclyl, wherein said non-aromatic heterocyclyl is optionally substituted on carbon with one or more R⁶⁰, and wherein any —NH— moiety of said non-aromatic heterocyclyl is optionally and independently substituted with R⁶⁰*;
   R⁷ is selected from halo, —CN, C₁₋₆alkyl, C₂₋₆alkenyl, C₂₋₆alkynyl, carbocyclyl, heterocyclyl, —OR^{7a}, —SR^{7a}, —N(R^{7a})₂, —N(R^{7a})—C(O)—R^{7b}, —N(R^{7a})—N(R^{7a})₂, —NO₂, —C(O)—H, —C(O)R^{7b}, —C(O)₂R^{7a}, —C(O)—N(R^{7a})₂, —O—C(O)—N(R^{7a})₂, —N(R^{7a})—C(O)₂R^{7a}, —N(R^{7a})—C(O)—N(R^{7a})₂, —O—C(O)—R^{7b}, —S(O)—R^{7b}, —S(O)₂—R^{7b}, —S(O)₂—N(R^{7a})₂, —N(R^{7a})—S(O)₂—R^{7b}, —C(R^{7a})═N—R^{7a}, and —C(R^{7a})═N—OR^{7a}, wherein said C₁₋₆alkyl, C₂₋₆alkenyl, C₂₋₆alkynyl, carbocyclyl, and heterocyclyl are optionally substituted on carbon with one or more R⁷⁰, and wherein any —NH— moiety of said heterocyclyl is optionally substituted with R⁷⁰*;
   R⁷* in each occurrence is independently selected from C₁₋₆alkyl, carbocyclyl, heterocyclyl, —C(O)—H, —C(O)—R^{7b}, —C(O)₂R^{7c}, —C(O)—N(R^{7a})₂, —S(O)—R^{7b}, —S(O)₂—R^{7b}, —S(O)₂—N(R^{7a})₂, —C(R^{7a})═N—R^{7a}, and —C(R^{7a})═N—OR^{7a}, wherein said C₁₋₆alkyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
   R^{7a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
   R^{7b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
   R^{7c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
   R¹⁰ in each occurrence is independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, N(R^{10a})—C(O)—R^{10b}, —N(R^{10a})—N(R^{10a})₂, —NO₂, —C(O)—H, —C(O)—R^{10b}, —C(O)₂R^{10a}, —C(O)—N(R^{10a})₂, —O—C(O)—N(R^{10a})₂, —N(R^{10a})—C(O)₂R^{10a}, —N(R^{10a})—C(O)—N(R^{10a})₂)—O—C(O)—R^{10b}, —S(O)—R^{10b}, —S(O)₂—R^{10b}, —S(O)₂—N(R^{10a})₂, —N(R^{10a})—S(O)₂—R^{10b}, —C(R^{10a})═N—R^{10a}, and —C(R^{10a})═N—OR^{10a};
   R¹⁰* in each occurrence is independently selected from C₁₋₆alkyl, carbocyclyl, heterocyclyl, —C(O)—H, —C(O)—R^{10b}, —C(O)₂R^{10c}, —C(O)—N(R^{10a})₂, —S(O)R^{10b}, —S(O)₂R^{10b}, —S(O)₂—N(R^{10a})₂, —C(R^{10a})═N—R^{10a}, and —C(R^{10a})═N—OR^{10a};
   R^{10a} in each occurrence is independently selected from H, C₁₋₆alkyl, carbocyclyl, and heterocyclyl;
   R^{10b} in each occurrence is independently selected from C₁₋₆alkyl, C₂₋₆alkenyl, C₂₋₆alkynyl, carbocyclyl, and heterocyclyl;
   R^{10c} in each occurrence is independently selected from C₁₋₆alkyl, carbocyclyl, and heterocyclyl;
   R²⁰ in each occurrence is independently selected from halo, —CN, C₁₋₆alkyl, C₂₋₆alkenyl, C₂₋₆alkynyl, carbocyclyl, heterocyclyl, —OR^{20a}, —SR^{20a}, —N(R^{20a})₂, —N(R^{20a})—C(O)—R^{20b}, —N(R^{20a})—N(R^{20a})₂, —NO₂, —C(O)—H, —C(O)—R^{20b}, —C(O)₂R^{20a}, —C(O)—N(R^{20a})₂, —O—C(O)—N(R^{20a})₂, —N(R^{20a})—C(O)₂R^{20a}, —N(R^{20a})—C(O)—N(R^{20a})₂, —O—C(O)—R^{20b}, —S(O)—R^{20b}, —S(O)₂—R^{20b}, —S(O)₂—N(R^{20a})₂, —N(R^{20a})—S(O)₂—R^{20b}, —C(R^{20a})═N—R^{20a}, and —C(R^{20a})═N—OR^{20a};
   R²⁰* in each occurrence is independently selected from C₁₋₆alkyl, carbocyclyl, heterocyclyl, —C(O)—H, —C(O)—R^{20b}, —C(O)₂R^{20c}, —C(O)—N(R^{20a})₂, —S(O)—R^{20b}, —S(O)₂—R^{20b}, —S(O)₂—N(R^{20a})₂, —C(R^{20a})═N—R^{20a}, and —C(R^{20a})═N—OR^{20a};
   R^{20a} in each occurrence is independently selected from H, C₁₋₆alkyl, carbocyclyl, and heterocyclyl;
   R^{20b} in each occurrence is independently selected from C₁₋₆alkyl, C₂₋₆alkenyl, C₂₋₆alkynyl, carbocyclyl, and heterocyclyl;
   R^{20c} in each occurrence is independently selected from C₁₋₆alkyl, C₂₋₆alkenyl, C₂₋₆alkynyl, carbocyclyl, and heterocyclyl;
   R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})—C(O)—R^{30b}, —N(R^{30a})—N(R^{30a})₂, —NO₂, —C(O)—H, —C(O)—R^{30b}, —C(O)₂R^{30a}, —C(O)—N(R^{30a})₂, —O—C(O)—N(R^{30a})₂, —N(R^{30a})—C(O)₂R^{30a}, —N(R^{30a})—C(O)—N(R^{30a})₂, —O—C(O)—R^{30b}), —S(O)—R^{30b}, —S(O)₂—R^{30b}, —S(O)₂—N(R^{30a})₂, —N(R^{30a})—S(O)₂—R^{30b}, —Si(R^{30b})₃, —C(R^{30a})═N—R^{30a}, and —C(R^{30a})═N—OR^{30a};
   R³⁰* in each occurrence is independently selected from C₁₋₆alkyl, carbocyclyl, heterocyclyl, —C(O)—H, —C(O)—R^{30b}, —C(O)₂R^{30c}, —C(O)—N(R^{30a})₂, —S(O)—R^{30b}, —S(O)₂—R^{30b}, —S(O)₂—N(R^{30a})₂, —C(R^{30a})═N—R^{30a}, and —C(R^{30a})═N—OR^{30a};
   R^{30a} in each occurrence is independently selected from H, C₁₋₆alkyl, carbocyclyl, and heterocyclyl;
   R^{30b} in each occurrence is independently selected from C₁₋₆alkyl, C₂₋₆alkenyl, C₂₋₆alkynyl, carbocyclyl, and heterocyclyl;
   R^{30c} in each occurrence is independently selected from C₁₋₆alkyl, C₂₋₆alkenyl, C₂₋₆alkynyl, carbocyclyl, and heterocyclyl;
   R⁴⁰ in each occurrence is independently selected from halo, —CN, C₁₋₆alkyl, C₂₋₆alkenyl, C₂₋₆alkynyl, carbocyclyl, heterocyclyl, —OR^{40a}, —SR^{40a}, —N(R^{40a})₂, —N(R^{40a})—C(O)—R^{40b}, —N(R^{40a})—N(R^{40a})₂, —NO₂, —C(O)—H, —C(O)—R^{40b}, —C(O)₂R^{40a}, —C(O)—N(R^{40a})₂, —O—C(O)—N(R^{40a})₂, —N(R^{40a})—C(O)₂R^{40a}, —N(R^{40a})—C(O)—N(R^{40a})₂, —O—C(O)—R^{40b}), —S(O)—R^{40b}, —S(O)₂—R^{40b}), —S(O)₂—N(R^{40a})₂, —N(R^{40a})—S(O)₂—R^{40b}, —C(R^{40a})═N—R^{40a}, and —C(R^{40a})═N—OR^{40a};
   R⁴⁰* in each occurrence is independently selected from C₁₋₆alkyl, carbocyclyl, heterocyclyl, —C(O)—H, —C(O)—R^{40b}, —C(O)₂R^{40c}, —C(O)—N(R^{40a})₂, —S(O)—R^{40b}, —S(O)₂—R^{40b}, —S(O)₂—N(R^{40a})₂, —C(R^{40a})═N—R^{40a}, and —C(R^{40a})═N—OR^{40a};
   R^{40a} in each occurrence is independently selected from H, C₁₋₆alkyl, carbocyclyl, and heterocyclyl
   R^{40b} in each occurrence is independently selected from C₁₋₆alkyl, C₂₋₆alkenyl, C₂₋₆alkynyl, carbocyclyl, and heterocyclyl;
   R^{40c} in each occurrence is independently selected from C₁₋₆alkyl, carbocyclyl, and heterocyclyl;
   R^{40x} in each occurrence is independently selected from halo, —CN, C₁₋₆alkyl, C₂₋₆alkenyl, C₂₋₆alkynyl, carbocyclyl, —OR^{40a}, —SR^{40a}, —N(R^{40a})₂, —N(R^{40a})—C(O)—R^{40b}, —N(R^{40a})—N(R^{40a})₂, —NO₂, —C(O)—H, —C(O)—R^{40b}, —C(O)₂R^{40a}, —C(O)—N(R^{40a})₂, —O—C(O)—N(R^{40a})₂, —N(R^{40a})—C(O)₂R^{40a}, —N(R^{40a})—C(O)—N(R^{40a})₂, —O—C(O)—R^{40b}, —S(O)—R^{40b}, —S(O)₂—R^{40b}, —S(O)₂—N(R^{40a})₂, —N(R^{40a})—S(O)₂—R^{40b}, —C(R^{40a})═N—R^{40a}, and —C(R^{40a})═N—OR^{40a};
   R⁵⁰ in each occurrence is independently selected from halo, —CN, C₁₋₆alkyl, C₂₋₆alkenyl, C₂₋₆alkynyl, carbocyclyl, heterocyclyl, —OR^{50a}, —SR^{50a}, —N(R^{50a})₂, —N(R^{50a})—C(O)—R^{50b}, —N(R^{50a})—N(R^{50a})₂, —NO₂, —C(O)—H, —C(O)—R^{50b}, —C(O)₂R^{50a}, —C(O)—N(R^{50a})₂, —O—C(O)—N(R^{50a})₂, —N(R^{50a})—C(O)₂R^{50a}, —N(R^{50a})—C(O)—N(R^{50a})₂, —O—C(O)—R^{50b}, —S(O)—R^{50b}, —S(O)₂—R^{50b}, —S(O)₂—N(R^{50a})₂, —N(R^{50a})—S(O)₂—R^{50b}, —Si(R^{50b})₃, —C(R^{50a})═N(R^{50a}), and —C(R^{50a})═N(OR^{50a});
   R⁵⁰* in each occurrence is independently selected from C₁₋₆alkyl, carbocyclyl, heterocyclyl, —C(O)—H, —C(O)—R^{50b}, —C(O)₂R^{50c}, —C(O)—N(R^{50a})₂, —S(O)—R^{50b}, —S(O)₂—R^{50b}, —S(O)₂—N(R^{50a})₂, —C(R^{50a})═N—R^{50a}, and —C(R^{50a})═N—OR^{50a};
   R^{50a} in each occurrence is independently selected from H, C₁₋₆alkyl, carbocyclyl, and heterocyclyl;
   R^{50b} in each occurrence is independently selected from C₁₋₆alkyl, C₂₋₆alkenyl, C₂₋₆alkynyl, carbocyclyl, and heterocyclyl;
   R^{50c} in each occurrence is independently selected from C₁₋₆alkyl, carbocyclyl, and heterocyclyl;
   R⁶⁰ in each occurrence is independently selected from halo, —CN, C₁₋₆alkyl, C₂₋₆alkenyl, C₂₋₆alkynyl, carbocyclyl, heterocyclyl, —OR^{60a}, —SR^{60a}, —N(R^{60a})₂, —N(R^{60a})—C(O)—R^{60b}, —N(R^{60a})—N(R^{60a})₂, —NO₂, —C(O)—H, —C(O)—R^{60b}, —C(O)₂R^{60a}, —C(O)—N(R^{60a})₂, —O—C(O)—N(R^{60a})₂, —N(R^{60a})—C(O)₂R^{60a}, —N(R^{60a})—C(O)—N(R^{60a})₂, —O—C(O)—R^{60b}), —S(O)—R^{60b}), —S(O)₂—R^{60b}, —S(O)₂—N(R^{60a})₂, —N(R^{60a})—S(O)₂—R^{60b}, —C(R^{60a})═N—R^{60a}, and —C(R^{60a})═N—OR^{60a};
   R⁶⁰* in each occurrence is independently selected from C₁₋₆alkyl, carbocyclyl, heterocyclyl, —C(O)—H, —C(O)—R^{60b}, —C(O)₂R^{60c}, —C(O)—N(R^{60a})₂, —S(O)—R^{60b}, —S(O)₂—R^{60b}, —S(O)₂—N(R^{60a})₂, —C(R^{60a})═N—R^{60a}, and —C(R^{60a})═N—OR^{60a};
   R^{60a} in each occurrence is independently selected from H, C₁₋₆alkyl, carbocyclyl, and heterocyclyl;
   R^{60b} in each occurrence is independently selected from C₁₋₆alkyl, C₂₋₆alkenyl, C₂₋₆alkynyl, carbocyclyl, and heterocyclyl;
   R^{60c} in each occurrence is independently selected from C₁₋₆alkyl, carbocyclyl, and heterocyclyl;
   R⁷⁰ in each occurrence is independently selected from halo, —CN, C₁₋₆alkyl, C₂₋₆alkenyl, C₂₋₆alkynyl, carbocyclyl, heterocyclyl, —OR^{70a}, —SR^{70a}, —N(R^{70a})₂, —N(R^{70a})—C(O)—R^{70b}, —N(R^{70a})—N(R^{70a})₂, —NO₂, —C(O)—H, —C(O)—R^{70b}, —C(O)₂R^{70a}, —C(O)—N(R^{70a})₂, —O—C(O)—N(R^{70a})₂, —N(R^{70a})—C(O)₂R^{70a}, —N(R^{70a})—C(O)—N(R^{70a})₂, —O—C(O)—R^{70b}, —S(O)—R^{70b}, —S(O)₂—R^{70b}, —S(O)₂—N(R^{70a})₂, —N(R^{70a})—S(O)₂—R^{70b}, —C(R^{70a})═N—R^{70a}, and —C(R^{70a})═N—R^{70a};
   R⁷⁰* in each occurrence is independently selected from C₁₋₆alkyl, carbocyclyl, heterocyclyl, —C(O)—H, —C(O)—R^{70b}, —C(O)₂R^{70c}, —C(O)—N(R^{70a})₂, —S(O)—R^{70b}, —S(O)₂—R^{70b}, —S(O)₂—N(R^{70a})₂, —C(R^{70a})═N—R^{70a}, and —C(R^{70a})═N—R^{70a};
   R^{70a} in each occurrence is independently selected from H, C₁₋₆alkyl, carbocyclyl, and heterocyclyl;
   R^{70b} in each occurrence is independently selected from C₁₋₆alkyl, C₂₋₆alkenyl, C₂₋₆alkynyl, carbocyclyl, and heterocyclyl;
   R^{70c} in each occurrence is independently selected from C₁₋₆alkyl, carbocyclyl, and heterocyclyl;
   W in each occurrence is independently selected from —O—, —S—, —N(R^{3a})—, —N(R^{3a})C(O)—, —C(O)—, —C(O)₂—, —C(O)—N(R^{3a})—, —O—C(O)—N(R^{3a})—, —N(R^{3a})—C(O)₂—, —S(O)—, —S(O)₂—, —S(O)₂—, and —N(R^{3a})—S(O)₂—; and
   X in each occurrence is independently selected from C₁₋₆alkylene, C₂₋₆alkenylene, and C₂₋₆alkynylene, wherein said C₁₋₆alkylene, C₂₋₆alkenylene, and C₂₋₆alkynylene, in each occurrence are optionally and independently substituted one or more R⁴⁰.

In another aspect, Ring A is a 6-membered non-aromatic heterocyclic ring, wherein
1) said 6-membered non-aromatic heterocyclic ring optionally contains, in addition to the nitrogen, a member selected from —O— and —NH—;
2) said 6-membered non-aromatic heterocyclic ring is optionally substituted on carbon with one or more R⁷; and
3) any —NH— moiety of said 6-membered ring is optionally substituted with R⁷*;
   Ring B is a 6-membered aromatic heterocyclic ring;
   n is 0;
   R¹ is selected from H and C₁₋₆alkyl;
   R² is selected from H and C₁₋₆alkyl;
   R⁴ in each occurrence is independently selected from H and halo.
   R⁷ is C₁₋₆alkyl;
   R⁷* in each occurrence is independently selected from H and —C(O)₂R^{7c}; and
   R^{7c} is C₁₋₆alkyl.

In still another aspect, Ring A is a 6-membered non-aromatic heterocyclic ring, wherein
1) said 6-membered non-aromatic heterocyclic ring optionally contains, in addition to the nitrogen, a member selected from —O— and —NH—;
2) said 6-membered non-aromatic heterocyclic ring is optionally substituted on carbon with one or more R⁷; and
3) any —NH— moiety of said 6-membered ring is optionally substituted with R⁷*;
   Ring B is pyridine;
   n is 0;
   R¹ is selected from H and C₁₋₆alkyl;
   R² is selected from H and C₁₋₆alkyl;
   R⁴ in each occurrence is independently selected from H and halo.
   R⁷ is C₁₋₆alkyl;
   R⁷* in each occurrence is independently selected from H and —C(O)₂R^{7c}; and
   R^{7c} is C₁₋₆alkyl.

In yet another aspect, Ring A is selected from morpholine, piperazine, and piperidine, wherein
1) said morpholine, piperazine, and piperidine are optionally substituted on carbon with one or more R⁷; and
2) any —NH— moiety of said piperidine is optionally substituted with R⁷*;
   Ring B is selected from:

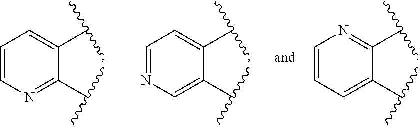

n is 0;
R¹ is selected from H and C₁₋₆alkyl;
R² is selected from H and C₁₋₆alkyl;
R⁴ in each occurrence is independently selected from H and halo.
R⁷ is C₁₋₆alkyl; and
R⁷* in each occurrence is independently selected from H and —C(O)₂R^{7c}; and
R^{7c} is C₁₋₆alkyl.

In a further aspect, Ring A is selected from 1-t-butoxycarbonylpiperazine, 2,6-dimethylmorpholine, 3,5-dimethylpiperidine piperidine, and piperazine;

Ring B, R³, R⁴, and n may together form a member selected from:

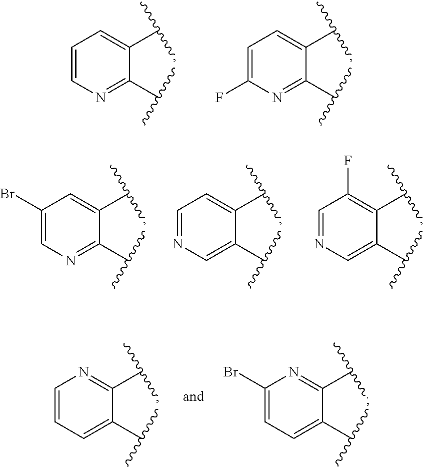

R¹ is selected from H and methyl; and
R² is selected from H and methyl.

In still a further aspect, Ring A is selected from morpholine, piperazine, and piperidine, wherein said morpholine, piperazine, and piperidine are optionally substituted on carbon with one or more R⁷, and wherein a —CH₂— group of said morpholine, piperazine, and piperidine can optionally be replaced by —C(O)—;

Ring B is pyridine;
n is 0 or 1;

### R¹ is H;

### R² is H;

R³ in each occurrence is independently selected from —X—R⁵ and —C(NH₂)═N—OH;
R⁴ in each occurrence is independently selected from H, —CN, halo, phenyl, and 5- or 6-membered heteroaryl, wherein said phenyl and 5- or 6-membered heteroaryl in each occurrence are optionally substituted with one or more R⁴⁰, and wherein any —NH— moiety of said 5- or 6-membered heteroaryl is optionally substituted with R⁴⁰*;
R⁵ in each occurrence is independently selected from phenyl and 5- or 6-membered heteroaryl, wherein said phenyl and 5- or 6-membered heteroaryl in each occurrence are optionally and independently substituted with one or more R⁵⁰;
R⁷ is C₁₋₆alkyl;
R⁴⁰ in each occurrence is independently selected from halo, C₁₋₆alkyl, phenyl, 5- or 6-membered heterocyclyl, —OR^{40a}, and —N(R^{40a})₂;
R⁴⁰* is C₁₋₆alkyl;
R^{40a} in each occurrence is independently selected from H and C₁₋₆alkyl;

### R⁵⁰ is −0 ea;

R^{50a} is C₁₋₆alkyl; and
X is ethyne-1,2-diyl.

In yet a further aspect, Ring A is selected from morpholine, piperazine, and piperidine, wherein said morpholine, piperazine, and piperidine are optionally substituted on carbon with one or more R⁷, and wherein a —CH₂— group of said morpholine, piperazine, and piperidine can optionally be replaced by —C(O)—;

Ring B is pyridine;
n is 0;

### R¹ is H;

### R² is H;

R⁴ in each occurrence is independently selected from H, —CN, halo, phenyl, and 5- or 6-membered heteroaryl, wherein said phenyl and 5- or 6-membered heteroaryl in each occurrence is optionally substituted with one or more R⁴⁰, and wherein any —NH— moiety of said 5- or 6-membered heteroaryl is optionally substituted with R⁴⁰*;
R⁷ is C₁₋₆alkyl;
R⁴⁰ in each occurrence is independently selected from halo, C₁₋₆alkyl, phenyl, 5- or 6-membered heterocyclyl, —OR^{40a}, and —N(R^{40a})₂;
R⁴⁰* is C₁₋₆alkyl; and
R^{40a} in each occurrence is independently selected from H and C₁₋₆alkyl.

In one aspect, Ring A is selected from morpholine, piperazine, and piperidine, wherein said morpholine, piperazine, and piperidine are optionally substituted on carbon with one or more R⁷, and wherein a —CH₂— group of said morpholine, piperazine, and piperidine can optionally be replaced by —C(O)—;

Ring B is pyridine;
n is 1;

### R¹ is H;

### R² is H;

R³ in each occurrence is independently selected from —X—R⁵ and —C(N(R^{3a})₂)═N—OR^{3y};

### R^{3a} is H;

### R^{3y} is H;

R⁴ in each occurrence is independently selected from H and halo;
R⁵ in each occurrence is independently selected from phenyl and 5- or 6-membered heteroaryl, wherein said phenyl and 5- or 6-membered heteroaryl in each occurrence are optionally and independently substituted with one or more R⁵⁰;
R⁷ is C₁₋₆alkyl;

### R⁵⁰ is —OR^{50a};

R^{50a} is C₁₋₆alkyl; and
X is ethyne-1,2-diyl.

In another aspect, Ring A is selected from 2,6-dimethylmorpholine, 3,5-dimethylpiperidine, 6-methylpiperazin-2-one, and piperidine;

Ring B is pyridine;
n is 0 or 1;

### R¹ is H;

### R² is H;

R³ is selected from —C(NH₂)═N—OH, 4-methoxyphenylethynyl, and pyrazin-2-ylethynyl; and
R⁴ in each occurrence is independently selected from H, —CN, bromo, chloro, fluoro, iodo, 1H-benzimidazol-2-yl, 1-benzofuran-2-yl, 1,3-benzothiazol-2-yl, 1-benzothien-2-yl, 5-chloropyridin-2-yl, 2-(dimethyylamino)pyrimidin-5-yl, 3,5-dimethylisoxazol-4-yl, 2,4-dimethyl-1,3-thiazol-5-yl, 4-fluorophenyl, furan-2-yl, furan-3-yl, 1H-imidazol-2-yl, 1H-imidazol-4-yl, 2-methoxyphenyl, 3-methyoxypyrazin-2-yl, 6-methoxypyrazin-2-yl, 4-methoxypyridin-3-yl, 2-methoxy-1,3-thiaol-4-yl, 1-methyl-1H-imidazol-2-yl, 1-methyl-1H-imidazol-4-yl, 1-methyl-1H-imidazol-5-yl, 2-methylphenyl, 1-methyl-1H-pyrazol-4-yl, 1-methyl-1H-pyrazol-5-yl, 1-methyl-1H-tetrazol-5-yl, 2-methyl-2H-tetrazol-5-yl, 5-methyl-1,3,4-thiadiazoly-2-yl, 3-methylthiophen-2-yl, 4-methylthiophen-3-yl, 5-methylthiophen-2-yl, 6-(morpholin-4-yl)pyridin-3-yl, 5-methyl-1,2,4-oxathiadiazol-3-yl, 1,3-oxazol-2-yl, phenyl, pyrazin-2-yl, 1H-pyrazol-4-yl, 1H-pyrazol-5-yl, 5-(1H-pyrazol-5-yl)thiophen-2-yl, pyridazin-4-yl, pyridin-2-yl, pyridin-3-yl, pyridin-4-yl, pyrimidin-2-yl, pyrimidin-5-yl, quinolin-2-yl, quinolin-8-yl, 1,3,4-thiadiazol-2-yl, 1,3-thiazol-2-yl, 1,3-thiazol-4-yl, thiazol-5-yl, thiophen-2-yl, and 5-(1H-tetrazol-5-yl)thiophen-2-yl.

In still a further aspect, the present invention provides a compound of Formula (I), or a pharmaceutically acceptable salt thereof, illustrated by the Examples, each of which provides a further independent aspect of the invention.

### Biological Activity

Typical compounds of Formula (I) are believed to inhibit bacterial DNA gyrase and are therefore of interest for their antibacterial effects. The inventive compounds are believed to be active against a variety of bacterial organisms, including both Gram positive and Gram negative aerobic and anaerobic bacteria.

These properties may be assessed using, for example, the testing methods shown below.

### Bacterial Susceptibility Testing Methods

Compounds may be tested for antimicrobial activity by susceptibility testing in liquid media in a 96 well format. Compounds may be dissolved in dimethylsulfoxide and tested in 10 doubling dilutions in the susceptibility assays. The organisms used in the assay may be grown overnight on suitable agar media and then suspended in a liquid medium appropriate for the growth of the organism. The suspension may be a 0.5 McFarland and a further 1 in 10 dilution may be advantageously made into the same liquid medium to prepare the final organism suspension in 100 μL. Plates may be incubated under appropriate conditions at 37° C. for 24 hours prior to reading. The Minimum Inhibitory Concentration (MIC) is intended to refer to the lowest drug concentration able to reduce growth by 80% or more. Compounds may be evaluated against organisms such as Gram-positive species, including Staphylococcus aureus, Streptococcus pneumoniae, Streptococcus pyogenes, and Enterococcus faecium; and Gram-negative species including Haemophilus influenzae, Escherichia coli and Moraxella catarrhalis.

Representative antibacterial activity for compounds of the instant invention is demonstrated by the table below.

### Antibacterial Activity

MIC (μg/mL)

| Bacteria | Example 18 | Example 19 |
|---|---|---|
| Hin737 | 32 | 16 |
| Hin446 | 16 | 4 |
| Hin158 | 8 | 1 |
| Eco524 | 32 | 2 |
| Mca445 | — | 8 |
| Sau517 | — | 32 |

### DNA Gyrase Supercoiling Activity Fluorescence Polarisation Assay

In a black, 384-well polystyrene assay plate, 30 microliters/well of 5 nM Escherichia coli DNA gyrase A/B tetramer and 130 micrograms/ml of topologically relaxed plasmid containing the triplex-forming sequence TTCTTCTTCTTCTTCTTCTTCTTCTTC in an assay buffer consisting of 35 mM Tris-HCl (pH 7.5), 24 mM KCl, 4 mM MgCl₂, 2 mM dithiothreitol, 1.8 mM spermidine, 5% (v/v) glycerol, 200 nM bovine serum albumin, 0.8% dimethylsulfoxide, and 0.3 mM ATP may be incubated at ambient temperature for (typically 30 minutes) in the absence or presence of 5-10 different concentrations of test compound. The supercoiling reactions may be quenched by the addition of 10 microliters/well of 40 nM oligodeoxynucleotide probe in 3× triplex-forming buffer consisting of 150 mM NaCl, and 150 mM sodium acetate at pH 3.5. The oligodeoxynucleotide probe may be 5′-BODIPY-FL-labeled TTCTTCTTC. After 60 minutes, the fluorescence anisotropy of the BODIPY-FL may be measured in a Tecan Ultra plate reader, using 485 nm excitation and 535 nm emission filters equipped with polarizers. The IC₅₀ may be determined by nonliner regression using two control reactions. The first contains no test compound but 0.8% DMSO (100% activity) while the second control reaction contains 5 uM Ciprofloxacin and 0.8% DMSO (0% activity).

When tested in an in-vitro assay based on the DNA gyrase supercoiling activity fluorescence polarisation assay described above, the E. coli DNA gyrase supercoiling IC₅₀ assay inhibitory activity of the following Examples was measured at the indicated IC₅₀. A dash indicates that an IC₅₀ was not provided for that particular compound.

### Examples 1 to 10

| Example | IC₅₀ (μM) |
|---|---|
| 1 | 7 |
| 2 | 9 |
| 3 | 11 |
| 4 | >100* |
| 5 | >100* |
| 6 | >83* |
| 7 | 3 |
| 8 | >83* |
| 9 | 3 |
| 10 | >83* |

### Examples 11 to 20

| Example | IC₅₀ (μM) |
|---|---|
| 11 | 30 |
| 12 | 21 |
| 13 | 25 |
| 14 | 11 |
| 15 | — |
| 16 | 7 |
| 17 | 6 |
| 18 | 51 |
| 19 | 11 |
| 20 | 25 |

### Examples 21 to 30

| Example | IC₅₀ (μM) |
|---|---|
| 21 | 10 |
| 22 | 4 |
| 23 | 6 |
| 24 | >5* |
| 25 | 9 |
| 26 | 4 |
| 27 | 5 |
| 28 | 2 |
| 29 | 9 |
| 30 | 12 |

### Examples 31 to 40

| Example | IC₅₀ (μM) |
|---|---|
| 31 | 6 |
| 32 | 24 |
| 33 | 48 |
| 34 | 15 |
| 35 | >40* |
| 36 | 2 |
| 37 | 12 |
| 38 | 12 |
| 39 | — |
| 40 | 6 |

### Examples 41 to 50

| Example | IC₅₀ (μM) |
|---|---|
| 41 | 5 |
| 42 | 4 |
| 43 | 12 |
| 44 | >10* |
| 45 | 30 |
| 46 | 10 |
| 47 | >83* |
| 48 | 17 |
| 49 | 30 |
| 50 | 8 |

### Examples 51 to 60

| Example | IC₅₀ (μM) |
|---|---|
| 51 | — |
| 52 | 5 |
| 53 | 16 |
| 54 | >83* |
| 55 | 21 |
| 56 | — |
| 57 | 9 |
| 58 | >83* |
| 59 | 50 |
| 60 | 6 |

### Examples 61 to 70

| Example | IC₅₀ (μM) |
|---|---|
| 61 | 18 |
| 62 | 8 |
| 63 | 35 |
| 64 | >83* |
| 65 | >10* |
| 66 | >83* |
| 67 | — |
| 68 | 8 |
| 69 | 29 |
| 70 | 3 |

### Examples 71 to 80

| Example | IC₅₀ (μM) |
|---|---|
| 71 | 16 |
| 72 | >5* |
| 73 | 3 |
| 74 | 4 |
| 75 | 8 |
| 76 | 21 |
| 77 | 8 |
| 78 | 2 |
| 79 | 17 |
| 80 | >5* |

### Examples 81 to 90

| Example | IC₅₀ (μM) |
|---|---|
| 81 | 8 |
| 82 | >83* |
| 83 | >83* |
| 84 | 7 |
| 85 | 8 |
| 86 | 61 |
| 87 | 4 |
| 88 | 2 |
| 89 | 11 |
| 90 | 21 |

### Examples 91 to 100

| Example | IC₅₀ (μM) |
|---|---|
| 91 | — |
| 92 | 20 |
| 93 | >83* |
| 94 | 7 |
| 95 | 2 |
| 96 | 3 |
| 97 | 44 |
| 98 | 51 |
| 99 | 44 |
| 100 | 7 |

### Examples 101 to 220

| Example | IC₅₀ (μM) |
|---|---|
| 101 | 2 |
| 102 | 16 |
| 103 | 4 |
| 104 | 22 |
| 105 | 19 |
| 106 | — |
| 107 | 6 |
| 108 | 6 |
| 109 | 14 |
| 110 | >83* |

### Examples 111 to 120(b)

| Example | IC₅₀ (μM) |
|---|---|
| 111 | 23 |
| 112 | >83* |
| 113 | 22 |
| 114 | 2 |
| 115 | >20* |
| 116 | 33 |
| 117 | 1 |
| 118 | 19 |
| 119 | 21 |
| 120(a) | 12 |
| 120(b) | 14 |

### Examples 121 to 123

| Example | IC₅₀ (μM) |
|---|---|
| 121 | 9 |
| 122 | 10 |
| 123 | 9 |

An asterisk indicates that in the particular assay used, no IC₅₀ was obtained for the designated Example at or below the indicated concentration.

In one aspect there is provided a compound of Formula (I), or a pharmaceutically acceptable salt thereof, for use as a medicament.

In one aspect, the terms “infection” and “bacterial infection” may refer to a bacterial infection caused by Acinetobacter baumanii. In another aspect, the terms “infection” and “bacterial infection” may refer to a bacterial infection caused by Aeromis hydrophila. In still another aspect, the terms “infection” and “bacterial infection” may refer to a bacterial infection caused by Bacillus anthracia. In yet another aspect, the terms “infection” and “bacterial infection” may refer to a bacterial infection caused by Bacteroides fragilis. In a further aspect, the terms “infection” and “bacterial infection” may refer to a bacterial infection caused by Bordatella pertussis. In still a further aspect, the terms “infection” and “bacterial infection” may refer to a bacterial infection caused by Burkholderia cepacia. In yet a further aspect, the terms “infection” and “bacterial infection” may refer to a bacterial infection caused by Chlamyida pneumoniae. In one aspect, the terms “infection” and “bacterial infection” may refer to a bacterial infection caused by Citrobacter freundii. In another aspect, the terms “infection” and “bacterial infection” may refer to a bacterial infection caused by Clostridium difficile. In still another aspect, the terms “infection” and “bacterial infection” may refer to a bacterial infection caused by Enterobacter cloacae. In yet another aspect, the terms “infection” and “bacterial infection” may refer to a bacterial infection caused by Enterococcus faecalis. In a further aspect, the terms “infection” and “bacterial infection” may refer to a bacterial infection caused by Enterococcus faecium. In still a further aspect, the terms “infection” and “bacterial infection” may refer to a bacterial infection caused by Enterobacter aerogenes. In yet a further aspect, the terms “infection” and “bacterial infection” may refer to a bacterial infection caused by Escherichia coli. In one aspect, the terms “infection” and “bacterial infection” may refer to a bacterial infection caused by Fusobacterium necrophorum. In another aspect, the terms “infection” and “bacterial infection” may refer to a bacterial infection caused by Haemophilus influenzae. In still another aspect, the terms “infection” and “bacterial infection” may refer to a bacterial infection caused by Haemophilus parainfluenzae. In yet another aspect, the terms “infection” and “bacterial infection” may refer to a bacterial infection caused by Haemophilus somnus. In a further aspect, the terms “infection” and “bacterial infection” may refer to a bacterial infection caused by Klebsiella oxytoca. In still a further aspect, the terms “infection” and “bacterial infection” may refer to a bacterial infection caused by Klebsiella pneumoniae. In yet a further aspect, the terms “infection” and “bacterial infection” may refer to a bacterial infection caused by Legionella pneumophila. In one aspect, the terms “infection” and “bacterial infection” may refer to a bacterial infection caused by Listeria monocytogenes. In another aspect, the terms “infection” and “bacterial infection” may refer to a bacterial infection caused by Moraxella catarrhalis. In still another aspect, the terms “infection” and “bacterial infection” may refer to a bacterial infection caused by Morganella morganii. In yet another aspect, the terms “infection” and “bacterial infection” may refer to a bacterial infection caused by Mycoplasma pneumoniae. In a further aspect, the terms “infection” and “bacterial infection” may refer to a bacterial infection caused by Neisseria gonorrhoeae. In still a further aspect, the terms “infection” and “bacterial infection” may refer to a bacterial infection caused by Neisseria meningitidis. In yet a further aspect, the terms “infection” and “bacterial infection” may refer to a bacterial infection caused by Pasteurella multocida. In one aspect, the terms “infection” and “bacterial infection” may refer to a bacterial infection caused by Proteus mirabilis. In another aspect, the terms “infection” and “bacterial infection” may refer to a bacterial infection caused by Proteus vulgaris. In still another aspect, the terms “infection” and “bacterial infection” may refer to a bacterial infection caused by Pseudomonas aeruginosa. In yet another aspect, the terms “infection” and “bacterial infection” may refer to a bacterial infection caused by Salmonella typhi. In a further aspect, the terms “infection” and “bacterial infection” may refer to a bacterial infection caused by Salmonella typhimurium. In still a further aspect, the terms “infection” and “bacterial infection” may refer to a bacterial infection caused by Serratia marcesens. In yet a further aspect, the terms “infection” and “bacterial infection” may refer to a bacterial infection caused by Shigella flexneria. In one aspect, the terms “infection” and “bacterial infection” may refer to a bacterial infection caused by Shigella dysenteriae. In another aspect, the terms “infection” and “bacterial infection” may refer to a bacterial infection caused by Staphylococcus aureus. In still another aspect, the terms “infection” and “bacterial infection” may refer to a bacterial infection caused by Staphylococcus epidermidis. In yet another aspect, the terms “infection” and “bacterial infection” may refer to a bacterial infection caused by Staphylococcus haemolyticus. In a further aspect, the terms “infection” and “bacterial infection” may refer to a bacterial infection caused by Staphylococcus intermedius. In still a further aspect, the terms “infection” and “bacterial infection” may refer to a bacterial infection caused by Staphylococcus saprophyticus. In yet a further aspect, the terms “infection” and “bacterial infection” may refer to a bacterial infection caused by Stenotrophomonas maltophila. In one aspect, the terms “infection” and “bacterial infection” may refer to a bacterial infection caused by Streptococcus agalactiae. In another aspect, the terms “infection” and “bacterial infection” may refer to a bacterial infection caused by Streptococcus mutans. In a still another aspect, the terms “infection” and “bacterial infection” may refer to a bacterial infection caused by Streptococcus pneumoniae. In yet another aspect, the terms “infection” and “bacterial infection” may refer to a bacterial infection caused by Streptococcus pyrogenes.

In one aspect, the terms “infection and “bacterial infection” may refer to a bacterial infection caused by a bacteria of the genus Aeromonas. In another aspect, the terms “infection and “bacterial infection” may refer to a bacterial infection caused by a bacteria of the genus Acinetobacter. In still another aspect, the terms “infection and “bacterial infection” may refer to a bacterial infection caused by a bacteria of the genus Bacillus. In yet another aspect, the terms “infection and “bacterial infection” may refer to a bacterial infection caused by a bacteria of the genus Bacteroides. In a further aspect, the terms “infection and “bacterial infection” may refer to a bacterial infection caused by a bacteria of the genus Bordetella. In still a further aspect, the terms “infection and “bacterial infection” may refer to a bacterial infection caused by a bacteria of the genus Burkholderia. In yet a further aspect, the terms “infection and “bacterial infection” may refer to a bacterial infection caused by a bacteria of the genus Chlamydophila. In one aspect, the terms “infection and “bacterial infection” may refer to a bacterial infection caused by a bacteria of the genus Citrobacter. In another aspect, the terms “infection and “bacterial infection” may refer to a bacterial infection caused by a bacteria of the genus Clostridium. In still another aspect, the terms “infection and “bacterial infection” may refer to a bacterial infection caused by a bacteria of the genus Enterobacter. In yet another aspect, the terms “infection and “bacterial infection” may refer to a bacterial infection caused by a bacteria of the genus Enterococcus. In a further aspect, the terms “infection and “bacterial infection” may refer to a bacterial infection caused by a bacteria of the genus Escherichia. In still a further aspect, the terms “infection and “bacterial infection” may refer to a bacterial infection caused by a bacteria of the genus Flavobacterium. In yet a further aspect, the terms “infection and “bacterial infection” may refer to a bacterial infection caused by a bacteria of the genus Fusobacterium. In one aspect, the terms “infection and “bacterial infection” may refer to a bacterial infection caused by a bacteria of the genus Haemophilus. In one aspect, the terms “infection and “bacterial infection” may refer to a bacterial infection caused by a bacteria of the genus Klebsiella. In another aspect, the terms “infection and “bacterial infection” may refer to a bacterial infection caused by a bacteria of the genus Legionella. In still another aspect, the terms “infection and “bacterial infection” may refer to a bacterial infection caused by a bacteria of the genus Listeria. In yet another aspect, the terms “infection and “bacterial infection” may refer to a bacterial infection caused by a bacteria of the genus Morganella. In a further aspect, the terms “infection and “bacterial infection” may refer to a bacterial infection caused by a bacteria of the genus Moraxella. In still a further aspect, the terms “infection and “bacterial infection” may refer to a bacterial infection caused by a bacteria of the genus Mycoplasma. In yet a further aspect, the terms “infection and “bacterial infection” may refer to a bacterial infection caused by a bacteria of the genus Neisseria. In one aspect, the terms “infection and “bacterial infection” may refer to a bacterial infection caused by a bacteria of the genus Pasteurella. In another aspect, the terms “infection and “bacterial infection” may refer to a bacterial infection caused by a bacteria of the genus Peptococci. In still another aspect, the terms “infection and “bacterial infection” may refer to a bacterial infection caused by a bacteria of the genus Peptostreptococci. In yet another aspect, the terms “infection and “bacterial infection” may refer to a bacterial infection caused by a bacteria of the genus Prevotella. In a further aspect, the terms “infection and “bacterial infection” may refer to a bacterial infection caused by a bacteria of the genus Proteus. In still a further aspect, the terms “infection and “bacterial infection” may refer to a bacterial infection caused by a bacteria of the genus Pseudomonas. In still another aspect, the terms “infection and “bacterial infection” may refer to a bacterial infection caused by a bacteria of the genus Salmonella. In yet a further aspect, the terms “infection and “bacterial infection” may refer to a bacterial infection caused by a bacteria of the genus Serratia. In one aspect, the terms “infection and “bacterial infection” may refer to a bacterial infection caused by a bacteria of the genus Shigella. In yet another aspect, the terms “infection and “bacterial infection” may refer to a bacterial infection caused by a bacteria of the genus Staphylococcus. In another aspect, the terms “infection and “bacterial infection” may refer to a bacterial infection caused by a bacteria of the genus Stenotrophomonas. In still another aspect, the terms “infection and “bacterial infection” may refer to a bacterial infection caused by a bacteria of the genus Streptococcus.

In one aspect, the terms “infection” and “bacterial infection” may refer to a gynecological infection. In another aspect the terms “infection” and “bacterial infection” may refer to a respiratory tract infection (RTI). In still another, the terms “infection” and “bacterial infection” may refer to a sexually transmitted disease. In yet another aspect, the terms “infection” and “bacterial infection” may refer to a urinary tract infection. In a further aspect, the terms “infection” and “bacterial infection” may refer to acute exacerbation of chronic bronchitis (ACEB). In yet a further aspect, the terms “infection” and “bacterial infection” may refer to acute otitis media. In one aspect, the terms “infection” and “bacterial infection” may refer to acute sinusitis. In another aspect, the terms “infection” and “bacterial infection” may refer to an infection caused by drug resistant bacteria. In still another aspect, the terms “infection” and “bacterial infection” may refer to catheter-related sepsis. In yet another aspect, the terms “infection” and “bacterial infection” may refer to chancroid. In a further aspect, the terms “infection” and “bacterial infection” may refer to chlamydia. In still a further aspect, the terms “infection” and “bacterial infection” may refer to community-acquired pneumonia (CAP). In yet a further aspect, the terms “infection” and “bacterial infection” may refer to complicated skin and skin structure infection. In one aspect, the terms “infection” and “bacterial infection” may refer to uncomplicated skin and skin structure infection. In another aspect, the terms “infection” and “bacterial infection” may refer to endocarditis. In still another aspect, the terms “infection” and “bacterial infection” may refer to febrile neutropenia. In yet another aspect, the terms “infection” and “bacterial infection” may refer to gonococcal cervicitis. In a further aspect, the terms “infection” and “bacterial infection” may refer to gonococcal urethritis. In still a further aspect, the terms “infection” and “bacterial infection” may refer to hospital-acquired pneumonia (HAP). In yet another aspect, the terms “infection” and “bacterial infection” may refer to osteomyelitis. In a further aspect, the terms “infection” and “bacterial infection” may refer to sepsis. In still a further aspect, the terms “infection” and “bacterial infection” may refer to syphilis.

In one aspect, there is provided the use of a compound of Formula (I), or a pharmaceutically acceptable salt thereof, in the manufacture of a medicament for the production of a bacterial DNA gyrase inhibitory effect, in a warm-blooded animal such as man.

In another aspect, there is provided the use a compound of Formula (I), or a pharmaceutically acceptable salt thereof, in the manufacture of a medicament for the treatment of a bacterial infection in a warm-blooded animal such as man.

In still another aspect, there is provided the use of a compound of Formula (I), or a pharmaceutically acceptable salt thereof, in the manufacture of a medicament for the treatment of urinary tract infections, pneumonia, prostatitis, skin and soft tissue infections, and intra-abdominal infections, in a warm-blooded animal such as man.

In yet another aspect, there is provided a method for producing a bacterial DNA gyrase inhibitory effect in a warm-blooded animal such as man, said method comprising administering to said animal an effective amount of a compound of Formula (I), or a pharmaceutically acceptable salt thereof.

In a further aspect, there is provided a method for treating a bacterial infection in a warm-blooded animal such as man, said method comprising administering to said animal an effective amount of a compound of formula (I), or a pharmaceutically acceptable salt thereof.

In still a further aspect, there is provided a method for treating urinary tract infections, pneumonia, prostatitis, skin and soft tissue infections, and intra-abdominal infections, in a warm-blooded animal such as man, said method comprising administering to said animal an effective amount of a compound of Formula (I), or a pharmaceutically acceptable salt thereof.

In yet a further aspect, there is provided a compound of Formula (I), or a pharmaceutically acceptable salt thereof, for use in producing a bacterial DNA gyrase inhibitory effect in a warm-blooded animal such as man.

In one aspect, there is provided a compound of Formula (I), or a pharmaceutically acceptable salt thereof, for use in treating a bacterial infection in a warm-blooded animal, such as man.

In another aspect, there is provided a compound of Formula (I), or a pharmaceutically acceptable salt thereof, for use in treating urinary tract infections, pneumonia, prostatitis, skin and soft tissue infections, and intra-abdominal infections, in a warm-blooded animal such as man.

In still another aspect, there is provided a pharmaceutical composition comprising a compound of Formula (I), or a pharmaceutically acceptable salt thereof, and at least one pharmaceutically acceptable carrier, diluent, or excipient.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients well known in the art. Thus, compositions intended for oral use may contain, for example, one or more coloring, sweetening, flavoring and/or preservative agents.

Suitable pharmaceutically acceptable excipients for a tablet formulation include, for example, inert diluents such as lactose, sodium carbonate, calcium phosphate or calcium carbonate; granulating and disintegrating agents such as corn starch or algenic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form or in the form of nano or micronized particles together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives such as ethyl or propyl p-hydroxybenzoate; anti-oxidants such as ascorbic acid); coloring agents; flavoring agents; and/or sweetening agents such as sucrose, saccharine or aspartame.

Oily suspensions may be formulated by suspending the active ingredient in a vegetable oil such as arachis oil, olive oil, sesame oil or coconut oil or in a mineral oil such as liquid paraffin. The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 4 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

In addition to the compounds of the present invention, the pharmaceutical composition of this invention may also contain or be co-administered (simultaneously, sequentially or separately) with one or more known drugs selected from other clinically useful classes of antibacterial agents (for example, macrolides, quinolones, β-lactams or aminoglycosides) and/or other anti-infective agents (for example, an antifungal triazole or amphotericin). These may include carbapenems, for example meropenem or imipenem, to broaden the therapeutic effectiveness. Compounds of this invention may also contain or be co-administered with bactericidal/permeability-increasing protein (BPI) products or efflux pump inhibitors to improve activity against gram negative bacteria and bacteria resistant to antimicrobial agents.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. Accordingly, the optimum dosage may be determined by the practitioner who is treating any particular patient.

In addition to its use in therapeutic medicine, the compound of Formulas (I) and its pharmaceutically acceptable salts are also useful as pharmacological tools in the development and standardization of in vitro and in vivo test systems for the evaluation of the effects of inhibitors of DNA gyrase in laboratory animals such as cats, dogs, rabbits, monkeys, rats and mice, as part of the search for new therapeutic agents.

If not commercially available, the necessary starting materials for the procedures such as those described herein may be made by procedures which are selected from standard organic chemical techniques, techniques which are analogous to the synthesis of known, structurally similar compounds, or techniques which are analogous to the described procedure or the procedures described in the Examples.

It is noted that many of the starting materials for synthetic methods as described herein are commercially available and/or widely reported in the scientific literature, or could be made from commercially available compounds using adaptations of processes reported in the scientific literature. The reader is further referred to Advanced Organic Chemistry, 5^{th} Edition, by Jerry March and Michael Smith, published by John Wiley & Sons 2001, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, published by John Wiley and Sons, 1991) and as described hereinabove.

Compounds of Formula (I) may be prepared in a variety of ways. Process A shown below illustrates a method for synthesizing compounds of Formula (I) (wherein Ring A, Ring B, R¹, R², R³, R⁴, and n, unless otherwise defined, are as defined hereinabove). The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents, which are compatible with the reaction conditions, will be readily apparent to one skilled in the art and alternate methods must then be used. The Scheme and Processes are not intended to present an exhaustive list of methods for preparing the compounds of Formula (I); rather, additional techniques of which the skilled chemist is aware may be also be used for the compounds' synthesis. The claims are not intended to be limited to the structures shown in the Schemes and Processes.

The skilled chemist will be able to use and adapt the information contained and referenced within the above references, and accompanying Examples therein and also the Examples and Scheme herein, to obtain necessary starting materials and products.

In one aspect, compounds of Formula (I), or pharmaceutically acceptable salts thereof, may be prepared by:

### Process A—Reacting a Compound of Formula (A1):

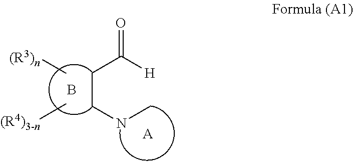

with a compound of Formula (A2):

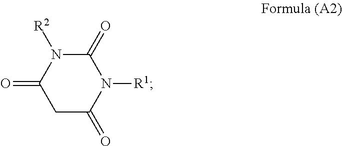

and thereafter if necessary:
i) converting a compound of Formula (I) into another compound of Formula (I);
ii) removing any protecting groups; and/or
iii) forming a pharmaceutically acceptable salt.

The reaction of Process A may be carried out in one or two separate reaction steps by reaction of a compound of Formula (A1) with a compound of Formula (A2) under standard Knoevenagel reaction conditions to form an intermediate olefin. Solvents suitable for such a reaction include alcohols such as methanol, isopropanol, and butanol, hydrocarbon solvents such as toluene and benzene and ethereal solvents such as dioxane and dimethoxyethane. Typical temperatures can range from about 60° C. to about 120° C. The Knoevenagel reaction may be catalyzed by a base such as triethylamine or pyrrolidine or an organic salt such as piperidinium acetate. Oftentimes under the reaction conditions, the intermediate olefin (Knoevenagel adduct) rearranges to a compound of Formula (A1), the rearrangement sometimes referred to as the “tertiary amine effect.” If the rearrangement does not occur, the temperature of the reaction may be increased and/or solvents can be exchanged to more polar solvents such as dimethylformamide and dimethylsulfoxide. Increased reaction temperature may then range from about 70° C. to about 180° C.

Scheme 1 depicts a procedure by which compounds of Formula (A1) may be prepared.

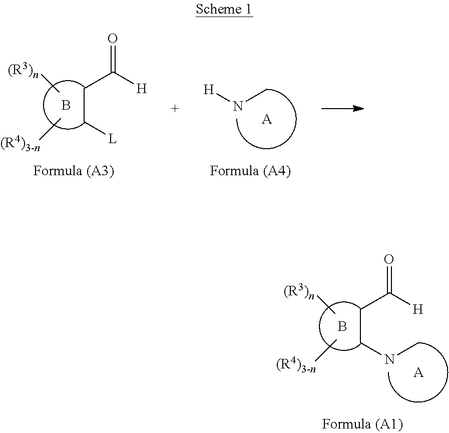

A compound of Formula (A3) may be reacted in a suitable solvent with a compound of Formula (A4) to provide a compound of Formula (A1). L is a leaving group, as defined hereinabove. The reaction may advantageously take place in the presence of an amine base, examples of which include triethylamine and diisopropylamine; an aromatic base, examples of which include pyridine, 4,6-dimethylpyridine, and dimethylaminopyridine; or an inorganic base, examples of which include sodium carbonate or potassium carbonate. Examples of suitable solvents include polar aprotic solvents such as acetonitrile, dimethylformamide, and dimethylsulfoxide; etheral solvents such as dioxane, tetrahydrofuran, and dimethoxyethane; or protic solvents, such as methanol and ethanol. The reaction may be performed at a temperature from about O° C. to about 150° C.

Compounds of Formula (A3) and (A4) are commercially available compounds, or they are known in the literature, or they are prepared by standard processes known in the art.

In any of the above-mentioned pharmaceutical compositions, processes, methods, uses, medicaments, and manufacturing features of the instant invention, any of the alternate embodiments of the compounds of the invention described herein also apply.

### EXAMPLES

The invention will now be further described with reference to the following illustrative examples in which, unless stated otherwise:
(i) temperatures are given in degrees Celsius (° C.); operations are carried out at room temperature or ambient temperature, that is, in a range of 18-25° C.;
(ii) organic solutions were dried over anhydrous magnesium sulfate; evaporation of organic solvent was carried out using a rotary evaporator under reduced pressure (4.5-30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC or liquid chromatography/mass spectroscopy (LC/MS) and reaction times are given for illustration only;
(v) final products have satisfactory proton nuclear magnetic resonance (NMR) spectra and/or mass spectra data;
(vi) yields are given for illustration only and are not necessarily those which can be obtained by diligent process development; preparations were repeated if more material was required;
(vii) when given, NMR data is in the form of delta values for major diagnostic protons, given in part per million (ppm) relative to tetramethylsilane (TMS) as an internal standard, determined at 300 MHz in DMSO-d₆ unless otherwise stated;
(viii) chemical symbols have their usual meanings;
(ix) solvent ratio was given in volume:volume (v/v) terms.
(x) the following abbreviations have been used:
   DMF N,N-dimethylformamide;
   THF tetrahydrofuran;
   DCM dichloromethane;
   DMAP 4-dimethylaminopyridine;
   DMSO dimethylsulphoxide;
   DIPEA N,N-diisopropylethylamine; and
   EtOAc ethyl acetate;
(xi) an ISCO Combiflash refers to flash chromatography on silica gel using Isco Combiflash® separation system: RediSep normal phase flash column, flow rate, 30-40 ml/min.
(xii) where a compound name is preceded by “rel”, as in, for example, “rel-(6aS,7S,9R)-1′,3′,7,9-Tetramethyl-6a,7,9,10-tetrahydro-1′H,5H-spiro[[1,4]oxazino[4,3-a][1,7]naphthyridine-6,5′-pyrimidine]-2′,4′,6′(3′H)-trione,” it is to be understood that such a designation implies that the indicated compound is present in the form of a racemic mixture.

### Intermediate 1

### 2-Bromo-5-fluoroisonicotinaldehyde

A solution of n-butyllithium, 2.5 M in hexanes (26.6 ml, 66.41 mmol) was added slowly to a solution of diisopropylamine (17.35 ml, 121.76 mmol) in THF (100 mL) cooled to below −70° C. The solution was warmed to −10° C. and re-cooled to below −70° C. A solution of 2-bromo-5-fluoropyridine (9.74 g, 53.4 mmol) in THF (15 mL) was added slowly keeping the temperature below −60° C. The mixture was stirred at −70° C. or below for 2 hours before DMF (8.14 ml, 105.16 mmol) was added slowly keeping the temperature below −60° C. After stirring for 1 hour at −70° C. or below, HCl, 4N in dioxane (55.3 ml, 221.38 mmol) was added slowly keeping the temperature below −60° C. After the addition was complete, the mixture was warmed to room temperature and diluted with EtOAc, before being washed with water and brine. The combined aqueous layers were extracted with additional EtOAc, which was washed with water and brine. The combined EtOAc layers were dried (MgSO₄) and concentrated to give a mobile liquid.

About ⅔ of the liquid was chromatographed on silica gel (100% hexanes followed by gradient elution to 100% CH₂Cl₂) to afford 7.75 g of the title compound as a solid.

¹H NMR (CDCl₃): 7.9 (s, 1H), 8.5 (s, 1H), 10.35 (s, 1H)

### Intermediate 2

### 4,6-Dichloropyridine-3-carbaldehyde

To the solution of methyl 4,6-dichloropyridine-3-carboxylate (20 g, 91.32 mmol) in CH₂Cl₂ (200 ml) at −78° C. was added DIBAL-H (1.5 N in CH₂Cl₂) (100.4 mmol, 64 ml) dropwise and stirred for 3 h at −78° C. The reaction mixture was quenched with 1.5 N HCl at −78° C. and was allowed to stir at room temperature for 1 hour. The reaction mixture was extracted with CH₂Cl₂, which was washed with brine and dried (NaSO₄). The solvent was removed and the residue was purified by flash column chromatography using ethyl acetate-petroleum ether as eluent to give product as a white solid. Yield: 12 g (73%).

MS (ES) MH⁺: 177.2 for C₆H₃Cl₂NO.

### Intermediate 3

### (5-Bromo-2-chloropyridin-3-yl)methanol

5-Bromo-2-chloronicotinic acid (1.0 g, 4.23 mmol) and thionyl chloride (50 ml, 685.04 mmol) were combined and heated to reflux. The reaction was stirred for 2 hours. The solution was cooled to room temperature and concentrated, chasing the anhydrous CH₂Cl₂. The resulting residue was added to a solution of sodium borohydride (0.576 g, 15.23 mmol) in water (50 ml) at 10° C. The reaction mixture was allowed to warm to room temperature and stir overnight. The reaction mixture was diluted with ethyl acetate and extracted twice with ethyl acetate. The combined organic extracts were dried over magnesium sulfate, filtered and evaporated.

MS (ES) MH⁺: 222 for C₆H₅BrClNO.

¹H NMR: 4.52 (d, 2H), 5.72 (t, 1H), 8.07 (s, 1H), 8.48 (s, 1H)

The following Intermediates were prepared by the procedure described in Intermediate 3 from the starting materials (SM) indicated.

### Intermediate 4

### (2,6-Difluoropyridin-3-yl)methanol

The title compound was prepared from 2,6-difluoronicotinic acid using a procedure similar to the one described for the synthesis of Intermediate 3.

MS (ES) MH⁺: 146 for C₆H₅F₂NO.

¹H NMR: 4.5 (d, 2H), 5.5 (t, 1H), 7.2 (d, 1H), 8.1 (dd, 1H).

### Intermediate 5

### 3-((2R,6S)-2,6-Dimethylmornholino)-5-fluoropicolinic acid

A solution of 3,5-difluoropicolinic acid (5.33 g, 33.50 mmol), cis-2,6-dimethylmorpholine (4.13 ml, 33.50 mmol) and DIEA (11.70 ml, 67.01 mmol) in THF (30 ml) was stirred at room temperature for 1 day. Additional cis-2,6-dimethylmorpholine (4.13 ml, 33.50 mmol) was added and the mixture was stirred at room temperature for 4 days. Additional cis-2,6-dimethylmorpholine (4.13 ml, 33.50 mmol) was added and the mixture was stirred at room temperature for an additional 2 days. Additional cis-2,6-dimethylmorpholine (2 ml) was added and the mixture was stirred at room temperature for another day. The mixture was diluted with water and brought to about pH=4 with 1N HCl. After saturating with NaCl, the aqueous solution was extracted 5 times with EtOAc and 5 times with THF. The combined organic layers were dried (MgSO₄) and concentrated to give 8.9 g of product as a white solid.

MS (ES) (M-H)⁻: 253 for C₁₂H₁₅FN₂O₃.

¹H NMR (DMSO-d⁶): 1.1 (d, 6H), 2.5 (m, 2H), 3.2 (d, 2H), 3.7 (m 2H), 7.5 (d, 1H), 8.1 (s, 1H), 13.3 (s, broad, 1H).

### Intermediate 6

### (3-((2R,6S)-2,6-Dimethylmorpholino)-5-fluoropyridin-2-yl)methanol

Ethyl chloroformate (2.9 ml, 30 mmol) was added to a solution of 3-((2R,6S)-2,6-dimethylmorpholino)-5-fluoropicolinic acid (Intermediate 5, 6.31 g, 24.82 mmol) and TEA (4.15 ml, 29.78 mmol) in 60 ml THF cooled in an ice water bath. After completion of the addition, the reaction mixture was allowed to warm to room temperature with stirring for 1 hour. Lithium borohydride (1.406 g, 64.53 mmol) was added portionwise and the mixture was stirred for 1 hour. The mixture was quenched first with water and then with 1N HCl. It was then taken up in EtOAc and aqueous Na₂CO₃. The organic layer was separated and washed with brine. The combined aqueous layers were extracted with EtOAc 3 times, each extract being washed with brine. The combined EtOAc layers were dried (MgSO₄) and concentrated to give an oil that slowly solidified. The material was chromatographed on silica gel (30% EtOAc in CH₂Cl₂ followed by gradient elution to 100% EtOAc) to give 2.85 g of the product as the major component.

MS (ES) MH⁺: 241 for C₁₂H₁₇FN₂O₂.

¹H NMR (DMSO-d⁶): 1.1 (d, 6H), 2.4 (t, 2H), 3.1 (d, 2H), 3.7-3.8 (m, 2H), 4.5 (d, 2H), 5.1 (t, 1H), 7.4 (d, 1H), 8.2 (s, 1H).

### Intermediate 7

### 5-Bromo-2-chloronicotinaldehyde

(5-Bromo-2-chloropyridin-3-yl)methanol (Intermediate 3, 0.67 g, 3.01 mmol) and pyridinium chlorochromate (0.779 g, 3.61 mmol) were combined in anhydrous dichloromethane (10 ml) and stirred at room temperature. The reaction was stirred for 2 hours. The reaction mixture was diluted with diethyl ether and filtered. The filtrate was concentrated to a tan solid. The solid was suspended in methanol and deposited onto Isolute and dried. Purification by normal phase Isco column (40%-100% dichloromethane/hexane) afforded the desired compound as a white solid (0.24 g).

MS (ES) MH⁺: 220 for C₆H₃BrClNO.

¹H NMR: 8.40 (s, 1H), 8.85 (s, 1H), 10.18 (s, 1H)

Intermediates 8 and 9 were prepared from the indicated starting materials using a procedure similar to the one described for the synthesis of Intermediate 7.

### Intermediate 8

### 2,6-Difluoro-5-(5-methyl-1,3,4-thiadiazol-2-yl)nicotinaldehyde

Starting Material: Intermediate 134.

MS (ES) MH⁺: 242 for C₉H₅F₂N₃OS.

¹H NMR: 2.9 (s, 3H), 9.2 (t, 1H), 10.2 (s, 1H).

### Intermediate 9

### 6-Bromo-4-chloro-3-((2R,6S)-2,6-dimethylmornholino)-5-fluoropicolinaldehyde

Starting Material: Intermediate 136.

MS (ES) MH⁺: 353 for C₁₂H₁₃BrClFN₂O₂.

¹H NMR: 1.2 (d, 6H), 2.9 (d, 2H), 3.05 (t, 1H), 3.9-4.0 (m, 2H), 9.95 (s, 1H).

### Intermediate 10

### 3-(Diethoxymethyl)-2,6-difluoropyridine

A solution of 2,6-difluoronicotinaldehyde (5 g, 34.94 mmol), triethyl orthoformate (8.72 ml, 52.41 mmol) and p-toluenesulfonic acid (0.562 mL, 3.49 mmol) in ethanol (50 mL) was heated at reflux overnight. Solvent was removed and the residue was diluted with EtOAc and washed with aqueous K₂CO₃ and brine. Drying (MgSO₄) and removal of solvent gave an oil. The oil was distilled under high vacuum conditions bulb-to-bulb to give 2.85 g of product as a clear, colorless oil.

¹H NMR: 1.25 (t, 3H), 3.5-3.7 (m 2H), 5.6 (s, 1H), 6.85 (d, 2H), 8.1 (dd, 1H).

### Intermediate 11

### (2R,6S)-4-(2-((tert-Butyldiphenylsilyloxy)methyl)-4-chloro-5-fluoropyridin-3-yl)-2,6-dimethylmorpholine

A solution of diisopropylamine (0.755 ml, 5.30 mmol) in THF (20 ml) was cooled in a dry ice-acetone bath. A solution of n-butyllithium (2.5 M in hexanes) (1.955 ml, 4.89 mmol) was added and the mixture was warmed to 0° C. and recooled in a dry ice-acetone bath. The solution was added to a second solution of (2R,6S)-4-(2-((tert-butyldiphenylsilyloxy)methyl)-5-fluoropyridin-3-yl)-2,6-dimethylmorpholine (Intermediate 141, 1.95 g, 4.07 mmol) in 15 ml THF cooled in a dry ice-acetone bath. The mixture was stirred for 10 min before the solution was transferred via cannula to a solution of hexachloroethane (0.6 ml, 5.3 mmol) in 15 ml THF also cooled in a dry ice-acetone bath. The mixture was allowed to warm to room temperature before being diluted with EtOAc and washed with water and brine. Combined aqueous layers were extracted again with EtOAc, which was washed with brine. Drying (MgSO₄) of the combined EtOAc extracts and removal of solvent gave a gummy solid that was chromatographed on silica gel (50% hexanes in CH₂Cl₂ followed by gradient elution to 100% CH₂Cl₂) to give 2 major components, the first eluting one (1.1 g) corresponding to desired product. MS (ES) MH⁺: 513 for C₂₈H₃₄ClFN₂O₂Si.

¹H NMR (CDCl₃): δ 1.0 (s, 9H), 1.15 (d, 6H, 2.4 (t, 2H), 3.0 (d, 2H), 3.6-3.7 (m, 2H), 4.8 (s, 2H), 7.0 (d, 1H), 7.4 (m, 6H), 7.7 (m, 4H), 8.2 (s, 1H).

### Intermediate 12

### 3-(Diethoxymethyl)-2,6-difluoro-5-iodopyridine

A solution of diisopropylamine (2.80 mL, 19.68 mmol) in tetrahydrofuran (30 mL) was cooled below −70° C. n-Butyllithium (6.30 mL, 15.74 mmol) was added slowly and the solution was warmed to room temperature before being recooled to below −70° C. A solution of 3-(diethoxymethyl)-2,6-difluoropyridine (Intermediate 10, 2.85 g, 13.12 mmol) in 10 ml THF was added slowly and the mixture was stirred with cooling by dry-ice/acetone for 90 minutes. A solution of iodine (4.00 g, 15.74 mmol) in 10 ml THF was added slowly and the mixture was slowly warmed to room temperature. The solution was treated with aqueous NaHSO₃ for 10 min before being diluted with EtOAc and washed with 1 N HCl and brine. Drying (MgSO₄) and removal of solvent gave an oil that slowly solidified affording 4.3 g of product. ¹H NMR: 1.25 (t, 3H), 3.5-3.7 (m 2H), 5.6 (s, 1H), 8.4 (s, 1H).

Intermediates 13 and 14 were prepared from the indicated starting materials using a procedure similar to the one described for the synthesis of Intermediate 12.

### Intermediate 13

### 3-((tert-Butyldiphenylsilyloxy)methyl)-2,6-difluoro-5-iodopyridine

Starting Material: Intermediate 140 and I₂.

MS (ES) MH⁺: 509 for C₂₂H₂₂F₂INOSi.

¹H NMR: 1.0 (s, 9H), 4.7 (s, 2H), 7.5 (m, 6H), 7.6 (m, 4H), 8.3 (t, 1H).

### Intermediate 14

### (2R,6S)-4-(6-Bromo-2-((tert-butyldiphenylsilyloxy)methyl)-4-chloro-5-fluoropyridin-3-yl)-2,6-dimethylmorpholine

Starting Material: Intermediate 11

MS (ES) MH⁺: 593 for C₂₈H₃₃.

BrClFN₂O₂Si; ¹H NMR (CDCl₃): 1.06 (s, 9H), 1.1 (d, 6H), 2.7 (m 2H), 3.1 (m, 2H), 3.6 (m 2H), 4.8 (s, 2H), 7.4 (m, 6H), 7.7 (m, 4H).

### Intermediate 15

### 2,6-Difluoro-5-iodonicotinaldehyde

A solution of 3-(diethoxymethyl)-2,6-difluoro-5-iodopyridine (Intermediate 12, 4.32 g, 12.59 mmol) and HCl (1N in water) (50 ml, 50.00 mmol) in THF (50 ml) was stirred at room temperature overnight. LC-MS shows incomplete reaction. Reaction was stirred at room temperature for another day. Work-up by diluting with water and treating with NaHCO₃. The mixture was extracted with EtOAc, which was washed with brine. The combined aqueous layers were extracted with EtOAc, which was washed with brine. The combined EtOAc layers were dried (MgSO₄) and concentrated to give a solid that was purified by chromatography on silica gel (50% hexanes in CH₂Cl₂ followed by gradient elution to 100% CH₂Cl₂) to afford 2.1 g of product.

¹H NMR: 8.7 (7, 1H), 10.2 (s, 1H).

### Intermediate 16

### 2-[(2R,6S)-2,6-Dimethylmornholin-4-yl]nicotinaldehyde

2-chloronicotinaldehyde (1.0 g, 7.1 mmol) was suspended in anhydrous acetonitrile. To this was added cis-2,6-dimethylmorpholine (1.3 mL, 10.6 mmol, Lancaster) and diisopropylethylamine (2.5 mL, 14.2 mmol). After heating at reflux for 18 hours, the reaction was cooled to room temperature and concentrated to a yellow oil which was purified by flash column (gradient elution 0-50% EtOAc/CH₂Cl₂) to yield pure product as a yellow oil (0.69 g).

MS (ES) MH⁺: 221 for C₁₂H₁₆N₂O₂.

¹H NMR: 1.1 (d, 6H) 2.7 (t, 2H) 3.7 (d, 4H) 7.0 (t, 1H), 8.1 (d, 1H), 8.4 (d, 1H), 9.9 (s, 1H).

Intermediates 17 to 30 were prepared from the indicated starting materials using a procedure similar to the one described for the synthesis of Intermediate 16.

### Intermediate 17

### 6-Bromo-3-((2R,6S)-2,6-dimethylmorpholino)picolinaldehyde

Starting Material: 6-Bromo-3-fluoropicolinaldehyde and (2R,6S)-dimethylmorpholine.

MS (ES) MH⁺: 300 for C₁₂H₁₅BrN₂O₂.

¹H NMR: 1.1 (d, 6H) 2.6 (t, 2H) 3.3 (t, 2H) 3.75 (d, 2H) 7.6 (d, 1H), 7.7 (d, 1H), 9.8 (s, 1H).

### Intermediate 18

### 2-Bromo-5-((2R,6S)-2,6-dimethylmorpholino)isonicotinaldehyde

Starting Material: 2-bromo-5-fluoroisonicotinaldehyde and (2R,6S)-2,6-dimethylmorpholine.

MS (ES) MH⁺: 299 for C₁₂H₁₅BrN₂O₂.

¹H NMR: 1.1 (d, 1H), 2.7 (t, 2H), 3.2 (d, 2H), 3.8 (m, 1H), 7.7 (s, 1H), 8.4 (s, 1H), 10.1 (s, 1H).

### Intermediate 19

### 2-Bromo-5-((3S,5R)-3,5-dimethylpiperidin-1-yl)isonicotinaldehyde

Starting Material: 2-bromo-5-fluoroisonicotinaldehyde and (2R,6S)-2,6-dimethylpiperidine.

MS (ES) MH⁺: 297 for C₁₂H₁₅BrN₂O₂.

¹H NMR: 1.1 (d, 1H), 2.7 (t, 2H), 3.2 (d, 2H), 3.8 (m, 1H), 7.7 (s, 1H), 8.4 (s, 1H), 10.1 (s, 1H).

### Intermediate 20

### 3-((2R,6S)-2,6-Dimethylmorpholino)-5-fluoroisonicotinaldehyde

Starting Material: 3,5-Difluoroisonicotinaldehyde and (2R,6S)-2,6-dimethylmorpholine.

MS (ES) MH⁺: 239 for C₁₂H₁₅FN₂O₂.

¹H NMR: 1.09 (d, 6H), 2.70 (t, 2H), 3.23 (d, 2H), 3.79 (m, 2H), 8.30 (s, 1H), 8.42 (s, 1H), 10.15 (s, 1H).

### Intermediate 21

### 3-Fluoro-5-(piperidin-1-yl)isonicotinaldehyde

Starting Material: 3,5-Difluoroisonicotinaldehyde and piperidine. MS (ES) MH⁺: 209 for C₁₁H₁₃FN₂O.

¹H NMR: 1.58 (m, 2H), 1.68 (m, 4H), 3.14 (m, 4H), 8.25 (s, 1H), 8.41 (s, 1H), 10.07 (s, 1H).

### Intermediate 22

### 2-(3,5-Dimethylpiperidin-1-yl)nicotinaldehyde

Starting Material: 2-chloronicotinaldehyde and 3,5-dimethylpiperidine.

MS (ES) MH⁺: 219 for C₁₃H₁₈N₂O.

¹H NMR: 0.75 (q, 1H), 0.9 (m, 6H), 1.8 (m, 2H), 1.8 (m, 1H), 3.1 (m, 1H), 3.4 (dd, 1H), 3.7 (d, 2H), 6.9 (dd, 1H), 8.0 (d, 1H), 8.3 (d, 1H), 9.9 (s, 1H).

### Intermediate 23

### 6-Bromo-3-(3,5-dimethylpiperidin-1-yl)picolinaldehyde

Starting Material: 6-Bromo-3-fluoropicolinaldehyde and 3,5-dimethylpiperidine.

MS (ES) MH⁺: 298 for C₁₃H₁₇BrN₂O.

¹H NMR: 0.7 (q, 1H), 0.8 (m, 6H), 1.4 (t, 1H), 1.8 (m, 3H), 2.1 (m, 1H), 2.4 (d, 1H), 2.8 (dd, 1H), 3.1 (dd, 1H), 3.3 (s, 1H), 7.6 (q, 2H), 9.8 (s, 1H).

### Intermediate 24

### 5-Bromo-2-((2S,6R)-2,6-dimethyl-morpholin-4-yl)-pyridine-3-carbaldehyde

Starting Material: Intermediate 7 and (2R,6S)-2,6-dimethylmorpholine.

MS (ES) MH⁺: 299 for C₁₂H₁₅BrN₂O₂.

¹H NMR: 1.1 (s, 6H), 2.7 (t, 2H), 3.7 (m, 4H), 8.2 (s, 1H), 8.45 (s, 1H), 9.85 (s, 1H).

### Intermediate 25

### 5-Bromo-2-(piperidin-1-yl)nicotinaldehyde

Starting Material: Intermediate 7 and piperidine.

MS (ES) MH⁺: 269 for C₁₁H₁₃BrN₂O

### Intermediate 26

### 5-Bromo-2-((3S,5R)-3,5-dimethylpiperidin-1-yl)nicotinaldehyde

Starting Material: Intermediate 7 and (3S,5R)-3,5-dimethylpiperidine hydrochloride.

MS (ES) MH⁺: 297 for C₁₃H₁₇BrN₂O.

### Intermediate 27

### 2-chloro-3-((2R,6S)-2,6-dimethylmorpholino)isonicotinaldehyde

Starting Material: 2-chloro-3-fluoroisonicotinaldehyde and (2R,6S)-2,6-dimethylmorpholine.

MS (ES) MH⁺: 255 for C₁₂H₁₅ClN₂O₂.

¹H NMR: 1.1 (d, 6H), 3.0 (m, 4H), 3.8 (m, 2H), 7.6 (s, 1H), 8.3 (s, 1H), 10.4 (s, 1H).

### Intermediate 28

### 2-chloro-3-((3S,5R)-3,5-dimethylpiperidin-1-yl)isonicotinaldehyde

Starting Material: 2-chloro-3-fluoroisonicotinaldehyde and (3S,5R)-3,5-dimethylpiperidine hydrochloride.

MS (ES) MH⁺: 253 for C₁₃H₁₇ClN₂O.

¹H NMR: 0.7 (q, 1H), 0.8 (d, 6H), 1.8 (m, 4H), 2.8 (t, 2H), 3.1 (d, 2H), 7.6 (d, 1H), 8.3 (d, 1H), 10.3 (s, 1H).

### Intermediate 29

### 2-chloro-3-(piperidin-1-yl)isonicotinaldehyde

Starting Material: 2-chloro-3-fluoroisonicotinaldehyde and piperidine.

MS (ES) MH⁺: 225 for C₁₁H₁₃ClN₂O.

¹H NMR: 1.6 (m, 6H), 3.2 (m, 4H), 7.5 (d, 1H), 8.3 (d, 1H), 10.4 (s, 1H).

### Intermediate 30

### 6-chloro-4-[(2R,6S)-2,6-dimethylmoryholin-4-yl]pyridine-3-carbaldehyde

Starting Material: Intermediate 2 and (2R,6S)-2,6-dimethylmorpholine.

MS (ES) MH⁺: 255.4 for C₁₂H₁₅ClN₂O₂.

¹H NMR (400 MHz, CDCl₃) δ: 1.2 (d, 6H), 2.75 (t, 2H), 3.3 (d, 2H), 3.9 (m, 2H), 6.8 (s, 1H), 8.5 (s, 1H), 9.9 (s, 1H).

### Intermediate 31

### 4-[2R,6S)-2,6-Dimethylmoryholin-4-yl]-6-(methylsulfanyl)pyridine-3-carbaldehyde

To a solution of 6-chloro-4-[(2R,6S)-2,6-dimethylmorpholin-4-yl]pyridine-3-carbaldehyde (Intermediate 30, 1.5 g, 5.9 mmol) in dioxane (20 ml) was added sodium thiomethoxide 21% solution in water (2.4 ml, 7.0 mmol) at 0° C. and the solution was heated to 100° C. for 14 hours. The solvent was removed under vacuum. The residue was dissolved in ethyl acetate, washed with water followed by brine and then dried over anhydrous sodium sulfate. It was filtered and the filtrate was evaporated under reduced pressure. The residue thus obtained was purified over silica gel using a gradient of ethyl acetate in petroleum ether to give product. Yield: 1.1 g, (75%).

MS (ES) MH⁺: 267.2 for C₁₃H₁₈N₂O₂S.

¹H NMR (400 MHz, CDCl₃) δ: 1.2 (d, 6H), 2.6 (s, 3H), 2.65 (m, 2H), 3.3 (d, 2H), 3.9 (m, 2H), 6.6 (s, 1H), 8.6 (s, 1H), 9.9 (s, 1H).

### Intermediate 32

### 4-[(2R,6S)-2,6-Dimethylmorpholin-4-yl]-6-methoxypyridine-3-carbaldehyde

To a solution of 6-chloro-4-[(2R,6S)-2,6-dimethylmorpholin-4-yl]pyridine-3-carbaldehyde (Intermediate 30, 3 g, 11.8 mmol) in methanol was added sodium methoxide (1.9 g, 35.5 mmol) at 0° C. and the solution was heated to 80° C. for 14 hours. The solvent was removed under vacuum. The residue was dissolved in ethyl acetate, washed with water followed by brine and then dried over anhydrous sodium sulfate. It was filtered and the filtrate was evaporated under reduced pressure. The residue thus obtained was purified over silica gel using a gradient of ethyl acetate in petroleum ether to give product. Yield 2.7 g, (87%).

MS (ES) MH⁺: 251.2 for C₁₃H₁₈N₂O₃.

¹H NMR (400 MHz, CDCl₃) δ: 1.2 (d, 6H), 2.65 (t, 2H), 3.3 (d, 2H), 3.9 (m, 3H), 4.0 (s, 3H), 6.1 (s, 1H), 8.4 (s, 1H), 9.9 (s, 1H).

### Intermediate 33

### 6-Fluoro-5-iodo-2-(3-methyl-5-oxopiperazin-1-yl)nicotinaldehyde

A solution of 6-methylpiperazin-2-one (233 mg, 2.04 mmol) in DMF (2 ml) was added slowly to a solution of 2,6-difluoro-5-iodonicotinaldehyde (Intermediate 15, 500 mg, 1.86 mmol) and 2,6-lutidine (0.238 ml, 2.04 mmol) in DMF (2 ml). The reaction was stirred at room temperature overnight. LC-MS shows mostly product MH⁺=364. The mixture was diluted with water and extracted EtOAc, which was washed with brine. Combined aqueous layers were 5 more times extracted with EtOAc, and each extract was washed with brine. Combined EtOAc layers were dried (MgSO₄) and concentrated to give an oil that partially solidified. Purification was carried out by reverse phase HPLC (35-50% CH₃CN in water gradient over 15 minutes) to afford 140 mg of product as a white solid.

MS (ES) MH⁺: 364 for C₁₁H₁₁FN₃O₂.

¹H NMR: 1.1 (d, 3H), 3.2 (m, 2H), 3.7 (m, 2H), 3.9 (s and m, total of 3H), 8.2 (s, 1H), 8.6 (d, 1H), 9.8 (s, 1H).

### Intermediate 34

### 2-((2R,6S)-2,6-Dimethylmornholino)-6-fluoro-5-(5-methyl-1,3,4-thiadiazol-2-yl)nicotinaldehyde

2,6-Difluoro-5-(5-methyl-1,3,4-thiadiazol-2-yl)nicotinaldehyde (Intermediate 8, 0.31 g, 1.3 mmol) and (2R,6S)-2,6-dimethylmorpholine (0.16 mL, 1.3 mmol) were combined in acetonitrile (10 mL). DIEA (0.22 mL, 1.3 mmol) was added and the reaction was stirred at room temperature for 5 minutes. LC/MS indicates starting material was consumed and there is a mixture of two regioisomers. The reaction mixture was diluted with water and extracted three times with ethyl acetate. The combined organic extracts were dried over magnesium sulfate, filtered and evaporated. TLC under various conditions gave no resolution of the two regioisomers. HPLC with gradient 20-50 ACN:H₂O gave separation of the two peaks. The sample was run on the Gilson with the same gradient as HPLC. The first test injection was 0.075 g on the small column and the remainder (0.306 g) was injected in a single injection on the large column. The second peak from the Gilson run was identified as desired product and the first peak was identified as the byproduct. The pure fractions were concentrated to yield product as a white solid (0.091 g, 21% yield).

MS (ES) MH⁺: 337 for C₁₅H₁₇FN₄O₂S.

¹H NMR: 1.1 (s, 6H), 2.8 (s, 3H), 2.9 (d, 2H), 3.6 (m, 2H), 3.9 (d, 2H), 9.0 (d, 1H), 9.9 (s, 1H).

### Intermediate 35

### 2-((3S,5R)-3,5-Dimethylpiperidin-1-yl)-6-fluoro-5-(5-methyl-1,3,4-thiadiazol-2-yl)nicotinaldehyde

The title compound was prepared from Intermediate using a procedure similar to the one described for the synthesis of Intermediate 34.

MS (ES) MH⁺: 335 for C₁₆H₁₉FN₄OS.

¹H NMR: 0.8 (m, 1H), 0.9 (d, 6H), 1.8 (m, 3H), 2.7 (dd, 2H), 2.8 (s, 3H), 3.9 (d, 2H), 8.9 (d, 1H), 9.9 (s, 1H).

### Intermediate 36

### 2-((2S,6R)-2,6-Dimethyl-mornholin-4-yl)-5-(4,4,5,5-tetramethyl[1,3,2,1]-dioxaborolan-2-yl)-pyridine-3-carbaldehyde

To a stirred and degassed solution of 5-bromo-2-(2S,6R)-2,6-dimethyl-morpholin-4-yl)-pyridine-3-carbaldehyde (Intermediate 24, 1.0 g, 3.3 mmol) in dioxane (10 mL) was added potassium acetate (1.63 g, 16.8 mmol), bis(pinacolato)diboron (1.73 g, 6.8 mmol), and 1,1′-Bis(diphenylphosphino)ferrocene palladiumdichloride-dichloromethane adduct (324 mg, 0.39 mmol), sequentially, and the mixture refluxed for 12 hours. It was cooled to room temperature, filtered through silica pad and concentrated. The residue was purified by silica get column using a gradient of ethylacetate in pet.ether to give product as off yellow solid. Yield: 2.0 g (86%).

MS (ES) MH⁺: 347 for C₁₈H₂₇BN₂O₄.

¹H NMR (300 MHz, CD₃OD) δ: 1.2-1.3 (m, 12H), 1.3 (s, 6H), 2.9 (t, 2H), 3.9 (d, 2H), 8.3 (s, 1H), 8.6 (s, 1H), 9.9 (s, 1H).

### Intermediate 37

### 6′-((2S,6R)-2,6-Dimethyl-mornholin-4-yl)-[2,3′]bipyridinyl-5′-carbaldehyde

To a de-gassed solution of 5-bromo-2-((2S,6R)-2,6-dimethyl-morpholin-4-yl)-pyridine-3-carbaldehyde (Intermediate 24, 250 mg, 0.83 mmol) in anhydrous THF (10 mL), was added 2-tri-n-butylstannyl pyridine (370 mg, 1.0 mmol) followed by dichlorobis(triphenylphosphine)-palladium(II) (60 mg, 0.08 mmol) under N₂. The reaction mixture heated at 75° C. for 14 h, cooled to room temperature, and solvent was removed. The residue was purified by flash chromatography over silica gel column using a gradient of ethyl acetate in petroleum ether to give product as a yellow solid. Yield: 110 mg (56%).

MS (ES) MH+: 298.3 for C₁₇H₁₉N₃O₂.

¹H NMR (300 MHz, DMSO-d₆) δ: 1.1 (m, 6H), 2.6 (t, 2H), 3.2 (d, 2H), 3.9 (t, 2H), 7.5 (d, 1H), 7.7 (d, 1H), 8.1 (d, 1H), 8.4 (d, 1H), 8.6 (d, 1H), 9.2 (s, 1H), 10.0 (s, 1H).

Intermediates 38 to 71 were prepared from the indicated starting material using a procedure similar to the one described for the synthesis of Intermediate 37.

### Intermediate 38

### 2-[(2R,6S)-2,6-Dimethylmornholin-4-yl]-5-(pyrimidin-2-yl)pyridine-3-carbaldehyde

Starting Material: Intermediate 24 and 2-tri-n-butylstannyl pyrimidine.

MS (ES) MH⁺: 299.2 for C₁₆H₁₈N₄O₂.

### Intermediate 39

### 2-[(2R,6S)-2,6-Dimethylmorpholin-4-yl]-5-(pyrazin-2-yl)pyridine-3-carbaldehyde

Starting Material: Intermediate 24 and 2-tri-n-butylstannyl pyrazine.

MS (ES) MH⁺: 299.2 for C₁₆H₁₈N₄O₂.

### Intermediate 40

### 5-[(2R,6S)-2,6-Dimethylmorpholin-4-yl]-2,2′-bipyridine-4-carbaldehyde

Starting Material: Intermediate 18 and 2-tri-n-butylstannyl pyridine.

MS (ES) MH⁺: 298.2 for C₁₇H₁₉N₃O₂.

### Intermediate 41

### 5-[(2R,6S)-2,6-Dimethylmorpholin-4-yl]-2-(pyrazin-2-yl)pyridine-4-carbaldehyde

Starting Material: Intermediate 18 and 2-tri-n-butylstannyl pyrazine.

MS (ES) MH⁺: 299.2 for C₁₆H₁₈N₄O₂.

### Intermediate 42

### 5-[(2R,6S)-2,6-Dimethylmorpholin-4-yl]-2,2′-bipyridine-6-carbaldehyde

Starting Material: Intermediate 17 and 2-tri-n-butylstannyl pyridine.

MS (ES) MH⁺: 298.2 for C₁₇H₁₉N₃O₂.

### Intermediate 43

### 3-[(2R,6S)-2,6-Dimethylmorpholin-4-yl]-6-(pyrazin-2-yl)pyridine-2-carbaldehyde Starting

Material: Intermediate 17 and 2-tri-n-butylstannyl pyrazine.

MS (ES) MH⁺: 299.2 for C₁₆H₁₈N₄O₂.

### Intermediate 44

### 2-[(2R,6S)-2,6-Dimethylmorpholin-4-yl]-5-(4-fluorophenyl)pyridine-3-carbaldehyde

Starting Material: Intermediate 24 and 4-tri-n-butylstannyl fluorobenzene.

MS (ES) MH⁺: 315.2 for C₁₈H₁₉FN₂O₂.

### Intermediate 45

### 3-[(2R,6S)-2,6-Dimethylmorpholin-4-yl]-6-(1,3-thiazol-2-yl)pyridine-2-carbaldehyde

Starting Material: Intermediate 17 and 2-tri-n-butylstannyl thiazole.

MS (ES) MH⁺: 304.2 for C₁₅H₁₇N₃O₂S.

### Intermediate 46

### 5-[(2R,6S)-2,6-Dimethylmorpholin-4-yl]-2-(1,3-thiazol-2-yl)pyridine-4-carbaldehyde

Starting Material: Intermediate 18 and 2-tri-n-butylstannyl thiazole.

MS (ES) MH⁺: 304.2 for C₁₅H₁₇N₃O₂S.

### Intermediate 47

### 5-(1,3-Benzothiazol-2-yl)-2-[(2R,6S)-2,6-Dimethylmorpholin-4-yl]pyridine-3-carbaldehyde

Starting Material: Intermediate 24 and 2-tri-n-butylstannyl benzothiazole.

MS (ES) MH⁺: 355.2 for C₁₉H₁₉N₃O₂S.

### Intermediate 48

### 6-(1,3-Benzothiazol-2-yl)-3-[(2R,6S)-2,6-Dimethylmorpholin-4-yl]pyridine-2-carbaldehyde

Starting Material: Intermediate 17 and 2-tri-n-butylstannyl benzothiazole.

MS (ES) MH⁺: 355.2 for C₁₉H₁₉N₃O₂S.

### Intermediate 49

### 2-(1,3-Benzothiazol-2-yl)-5-[(2R,6S)-2,6-Dimethylmorpholin-4-yl]pyridine-4-carbaldehyde

Starting Material: Intermediate 18 and 2-tri-n-butylstannyl benzothiazole.

MS (ES) MH⁺: 355.2 for C₁₉H₁₉N₃O₂S.

### Intermediate 50

### 2-[(2R,6S)-2,6-Dimethylmorpholin-4-yl]-5-(1-methyl-1H-imidazol-5-yl)pyridine-3-carbaldehyde

Starting Material: Intermediate 24 and 5-tri-n-butylstannyl-1-methyl-1H-imidazole.

MS (ES) MH⁺: 301.1 for C₁₆H₂₀N₄O₂.

### Intermediate 51

### 3-[(2R,6S)-2,6-Dimethylmorpholin-4-yl]-6-(1-methyl-1H-imidazol-5-yl)pyridine-2-carbaldehyde

Starting Material: Intermediate 17 and 5-tri-n-butylstannyl-1-methyl-1H-imidazole.

MS (ES) MH⁺: 301.1 for C₁₆H₂₀N₄O₂.

### Intermediate 52

### 2-[(2R,6S)-2,6-Dimethylmorpholin-4-yl]-5-(3-methoxypyrazin-2-yl)pyridine-3-carbaldehyde

Starting Material: Intermediate 24 and 2-tri-n-butylstannyl-3-methoxypyrazine.

MS (ES) MH⁺: 329.1 for C₁₇H₂₀N₄O₃.

### Intermediate 53

### 5-[(2R,6S)-2,6-Dimethylmorpholin-4-yl]-2-(3-methoxypyrazin-2-yl)pyridine-4-carbaldehyde

Starting Material: Intermediate 18 and 2-tri-n-butylstannyl-3-methoxypyrazine.

MS (ES) MH⁺: 329.1 for C₁₇H₂₀N₄O₃.

### Intermediate 54

### 2-[(2R,6S)-2,6-Dimethylmorpholin-4-yl]-5-(pyridazin-4-yl)pyridine-3-carbaldehyde

Starting Material: Intermediate 24 and 4-tri-n-butylstannyl pyridazine.

MS (ES) MH⁺: 299.2 for C₁₆H₁₈N₄O₂.

### Intermediate 55

### 3-[(2R,6S)-2,6-Dimethylmorpholin-4-yl]-6-(pyridazin-4-yl)pyridine-2-carbaldehyde

Starting Material: Intermediate 17 and 4-tri-n-butylstannyl pyridazine.

MS (ES) MH⁺: 299.2 for C₁₆H₁₈N₄O₂.

### Intermediate 56

### 5-[(2R,6S)-2,6-Dimethylmorpholin-4-yl]-2-(pyridazin-4-yl)pyridine-4-carbaldehyde

Starting Material: Intermediate 18 and 4-tri-n-butylstannyl pyridazine.

MS (ES) MH⁺: 299.2 for C₁₆H₁₈N₄O₂.

### Intermediate 57

### 2-[(2R,6S)-2,6-Dimethylmorpholin-4-yl]-5-(1,3-oxazol-2-yl)pyridine-3-carbaldehyde

Starting Material: Intermediate 24 and 2-tri-n-butylstannyl oxazole.

MS (ES) MH⁺: 288.2 for C₁₅H₁₇N₃O₃.

### Intermediate 58

### 3-[(2R,6S)-2,6-Dimethylmorpholin-4-yl]-6-(1,3-oxazol-2-yl)pyridine-2-carbaldehyde

Starting Material: Intermediate 17 and 2-tri-n-butylstannyl oxazole.

MS (ES) MH⁺: 288.2 for C₁₅H₁₇N₃O₃.

### Intermediate 59

### 5-[(2R,6S)-2,6-Dimethylmorpholin-4-yl]-2-(1,3-oxazol-2-yl)pyridine-4-carbaldehyde

Starting Material: Intermediate 18 and 2-tri-n-butylstannyl oxazole.

MS (ES) MH⁺: 288.2 for C₁₅H₁₇N₃O₃.

### Intermediate 60

### 2-[(2R,6S)-2,6-Dimethylmorpholin-4-yl]-5-(1,3-thiazol-4-yl)pyridine-3-carbaldehyde

Starting Material: Intermediate 24 and 4-tri-n-butylstannyl-1,3-thiazole.

MS (ES) MH⁺: 304.2 for C₁₅H₁₇N₃O₂S.

### Intermediate 61

### 3-[(2R,6S)-2,6-Dimethylmorpholin-4-yl]-6-(1,3-thiazol-4-yl)pyridine-2-carbaldehyde

Starting Material: Intermediate 17 and 4-tri-n-butylstannyl-1,3-thiazole.

MS (ES) MH⁺: 304.2 for C₁₅H₁₇N₃O₂S.

### Intermediate 62

### 5-[(2R,6S)-2,6-Dimethylmorpholin-4-yl]-2-(1,3-thiazol-4-yl)pyridine-4-carbaldehyde

Starting Material: Intermediate 18 and 4-tri-n-butylstannyl-1,3-thiazole.

MS (ES) MH⁺: 304.2 for C₁₅H₁₇N₃O₂S.

### Intermediate 63

### 2-[(2R,6S)-2,6-Dimethylmorpholin-4-yl]-5-(6-methoxypyrazin-2-yl)pyridine-3-carbaldehyde

Starting Material: Intermediate 24 and 2-tri-n-butylstannyl-6-methoxypyrazine.

MS (ES) MH⁺: 329.1 for C₁₇H₂₀N₄O₃.

### Intermediate 64

### 3-[(2R,6S)-2,6-Dimethylmorpholin-4-yl]-6-(6-methoxypyrazin-2-yl)pyridine-2-carbaldehyde

Starting Material: Intermediate 17 and 2-tri-n-butylstannyl-6-methoxypyrazine.

MS (ES) MH⁺: 329.1 for C₁₇H₂₀N₄O₃.

### Intermediate 65

### 5-[(2R,6S)-2,6-Dimethylmorpholin-4-yl]-2-(6-methoxypyrazin-2-yl)pyridine-4-carbaldehyde

Starting Material: Intermediate 18 and 2-tri-n-butylstannyl-6-methoxypyrazine.

MS (ES) MH⁺: 329.1 for C₁₇H₂₀N₄O₃.

### Intermediate 66

### 5-[2-(Dimethylamino)pyrimidin-5-yl]-2-[(2R,6S)-2,6-dimethylmorpholin-4-yl]pyridine-3-carbaldehyde

Starting Material: Intermediate 24 and 5-tri-n-butylstannyl-2-(dimethylamino)pyrimidine.

MS (ES) MH⁺: 452.2 for C₁₈H₂₃N₅O₂.

### Intermediate 67

### 2-[(2R,6S)-2,6-Dimethylmorpholin-4-yl]-5-(2-methoxy-1,3-thiazol-4-yl)pyridine-3-carbaldehyde

Starting Material: Intermediate 24 and 4-tri-n-butylstannyl-2-methoxy-1,3-thiazole.

MS (ES) MH⁺: 334.2 for C₁₆H₁₉N₃O₃S.

### Intermediate 68

### 2-[(2R,6S)-2,6-Dimethylmorpholin-4-yl]-5-(2,5-dimethyl-1,3-thiazol-4-yl)pyridine-3-carbaldehyde

Starting Material: Intermediate 24 and 4-tri-n-butylstannyl-2,5-dimethyl-1,3-thiazole.

MS (ES) MH⁺: 332.2 for C₁₇H₂N₃O₂S;

### Intermediate 69

### 3-[(2R,6S)-2,6-Dimethylmorpholin-4-yl]-6-(2,5-dimethyl-1,3-thiazol-4-yl)pyridine-2-carbaldehyde

Starting Material: Intermediate 17 and 4-tri-n-butylstannyl-2,5-dimethyl-1,3-thiazole. MS (ES) MH⁺: 332.2 for C₁₇H₂₁N₃O₂S.

### Intermediate 70

### 6-[(2R,6S)-2,6-Dimethylmorpholin-4-yl]-6′-(morpholin-4-yl)-3,3′-bipyridine-5-carbaldehyde

Starting Material: Intermediate 24 and 3-tri-n-butylstannyl-6-morpholinepyridine.

MS (ES) MH⁺: 383.2 for C₂₁H₂₆N₄O₃.

### Intermediate 71

### 5-[(2R,6S)-2,6-Dimethylmorpholin-4-yl]-6′-(morpholin-4-yl)-2,3′-bipyridine-4-carbaldehyde

Starting Material: Intermediate 18 and 3-tri-n-butylstannyl-6-morpholinepyridine.

MS (ES) MH⁺: 383.2 for C₂₁H₂₆N₄O₃.

### Intermediate 72

### 5-(1-Benzofuran-2-yl)-2-[(2R,6S)-2,6-dimethylmorpholin-4-yl]pyridine-3-carbaldehyde

To a stirred and degassed solution of 5-bromo-2-[(2R,6S)-2,6-dimethylmorpholin-4-yl]pyridine-3-carbaldehyde (Intermediate 24, 250 mg, 0.83 mmol) in acetonitrile:water (4:1) mixture (10 mL), was added sodium carbonate (65 mg, 0.61 mmol), benzofuran-2-boronic acid (153 mg, 0.91 mmol), 2-dicyclohexylphosphino 2′,4′6′-triisopropylbiphenyl (X-phos) (88 mg, 0.18 mmol) and tris(dibenzylideneacetone) dipalladium(0) (56 mg, 0.061 mmol), sequentially and the reaction mixture was heated to 85° C. for 12 hours. The reaction mixture was cooled to room temperature and concentrated. The residue thus obtained was purified over silica gel column using a gradient of ethyl acetate in pet. ether to give product as yellow solid. Yield: 169 mg (63%).

MS (ES) MH⁺: 337.2 for C₂₀H₂₀N₂O₃.

Intermediates 73 to 109 were prepared from the indicated starting materials using a procedure similar to the one described for the synthesis of Intermediate 72.

### Intermediate 73

### 2-[(2R,6S)-2,6-Dimethylmorpholin-4-yl]-5-(1-methyl-1H-pyrazol-5-yl)pyridine-3-carbaldehyde

Starting Material: Intermediate 24 and 1-methyl-1H-pyrazole-5-boronic acid pinacol ester.

MS (ES) MH⁺: 337.2 for C₂₀H₂₀N₂O₃.

### Intermediate 74

### 5-chloro-6′-[(2R,6S)-2,6-Dimethylmorpholin-4-yl]-2,3′-bipyridine-5′-carbaldehyde

Starting Material: Intermediate 24 and 5-chloropyridine-2-boronic acid.

MS (ES) MH⁺: 332.8 for C₁₇H₁₈ClN₃O₂.

### Intermediate 75

### 6-[(2R,6S)-2,6-Dimethylmorpholin-4-yl]-4′-methoxy-3,3′-bipyridine-5-carbaldehyde

Starting Material: Intermediate 24 and 4-methoxy-pyridine-3-boronic acid.

MS (ES) MH⁺: 328.2 for C₁₈H₂₁N₃O₃.

### Intermediate 76

### 2-[(2R,6S)-2,6-Dimethylmorpholin-4-yl]-5-(1H-pyrazol-4-yl)pyridine-3-carbaldehyde

Starting Material: Intermediate 24 and pyrazole-4-boronic acid.

MS (ES) MH⁺: 287.4 for C₁₅H₁₈N₄O₄.

### Intermediate 77

### 2-[(2R,6S)-2,6-Dimethylmorpholin-4-yl]-5-(pyrimidin-5-yl)pyridine-3-carbaldehyde

Starting Material: Intermediate 24 and pyrimidine-5-boronic acid.

MS (ES) MH⁺: 299.2 for C₁₆H₁₈N₄O₂.

### Intermediate 78

### 6-[(2R,6S)-2,6-Dimethylmorpholin-4-yl]-3,3′-bipyridine-5-carbaldehyde

Starting Material: Intermediate 24 and pyridine-3-boronic acid.

MS (ES) MH⁺: 298.2 for C₁₇H₁₉N₃O₂.

### Intermediate 79

### 2-[(2R,6S)-2,6-Dimethylmorpholin-4-yl]-5-(quinolin-8-yl)pyridine-3-carbaldehyde

Starting Material: Intermediate 24 and quinoline-8-boronic acid.

MS (ES) MH⁺: 348.2 for C₂₁H₂₁N₃O₂.

### Intermediate 80

### 2-[(2R,6S)-2,6-Dimethylmorpholin-4-yl]-5-(furan-3-yl)pyridine-3-carbaldehyde

Starting Material: Intermediate 24 and furan-3-boronic acid.

MS (ES) MH⁺: 287.4 for C₁₆H₁₈N₂O₃.

### Intermediate 81

### 5-(3,5-Dimethylisoxazol-4-yl)-2-[(2R,6S)-2,6-Dimethylmorpholin-4-yl]pyridine-3-carbaldehyde

Starting Material: Intermediate 24 and 3,5-dimethylisoxazole-4-boronic acid.

MS (ES) MH⁺: 316.2 for C₁₇H₂₁N₃O₃.

### Intermediate 82

### 6-[(2R,6S)-2,6-Dimethylmorpholin-4-yl]-3,4′-bipyridine-5-carbaldehyde

Starting Material: Intermediate 24 and pyridine-4-boronic acid.

MS (ES) MH⁺: 298.2 for C₁₇H₁₉N₃O₂.

### Intermediate 83

### 2-[(2R,6S)-2,6-Dimethylmorpholin-4-yl]-5-(1-methyl-1H-pyrazol-4-yl)pyridine-3-carbaldehyde

Starting Material: Intermediate 24 and 1-methyl-1H-pyrazole-4-boronic acid pinacol ester.

MS (ES) MH⁺: 301.2 for C₁₆H₂₀N₄O₂.

### Intermediate 84

### 2-(1-Benzofuran-2-yl)-5-[(2R,6S)-2,6-Dimethylmorpholin-4-yl]pyridine-4-carbaldehyde

Starting Material: Intermediate 18 and Benzo[B]furan-2-boronic acid.

MS (ES) MH⁺: 337.2 for C₂₀H₂₀N₀O₃.

### Intermediate 85

### 5-[(2R,6S)-2,6-Dimethylmorpholin-4-yl]-2-(furan-2-yl)pyridine-4-carbaldehyde

Starting Material: Intermediate 18 and 2-Furanboronic acid.

MS (ES) MH⁺: 287.2 for C₁₆H₁₈N₂O₃.

### Intermediate 86

### 5-[(2R,6S)-2,6-Dimethylmorpholin-4-yl]-2-(1-methyl-1H-pyrazol-5-yl)pyridine-4-carbaldehyde

Starting Material: Intermediate 18 and 1-Methyl-1H-pyrazole-5-boronic acid pinacol ester.

MS (ES) MH⁺: 301.2 for C₁₆H₂₀N₄O₂.

### Intermediate 87

### 5-[(2R,6S)-2,6-Dimethylmorpholin-4-yl]-4′-methoxy-2,3′-bipyridine-4-carbaldehyde

Starting Material: Intermediate 18 and 4-methoxy-3-boronic acid.

MS (ES) MH⁺: 328.2 for C₁₈H₂₁N₃O₃.

### Intermediate 88

### 5-[(2R,6S)-2,6-Dimethylmorpholin-4-yl]-2-(1H-pyrazol-4-yl)pyridine-4-carbaldehyde

Starting Material: Intermediate 18 and pyrazole-4-boronic acid.

MS (ES) MH⁺: 287.2 for C₁₅H₁₈N₄O₂.

### Intermediate 89

### 5-[(2R,6S)-2,6-Dimethylmorpholin-4-yl]-2-(pyrimidin-5-yl)pyridine-4-carbaldehyde

Starting Material: Intermediate 18 and pyrimidine-5-boronic acid.

MS (ES) MH⁺: 299.2 for C₁₆H₁₈N₄O₂.

### Intermediate 90

### 5-[(2R,6S)-2,6-Dimethylmorpholin-4-yl]-2,3′-bipyridine-4-carbaldehyde

Starting Material: Intermediate 18 and pyridine-3-boronic.

MS (ES) MH⁺: 298.2 for C₁₇H₁₉N₃O₂.

### Intermediate 91

### 5-[(2R,6S)-2,6-Dimethylmorpholin-4-yl]-2-(quinolin-8-yl)pyridine-4-carbaldehyde

Starting Material: Intermediate 18 and quinoline-8-boronic acid.

MS (ES) MH⁺: 348.2 for C₂₁H₂₁N₃O₂.

### Intermediate 92

### 5-[(2R,6S)-2,6-Dimethylmorpholin-4-yl]-2-(furan-3-yl)pyridine-4-carbaldehyde

Starting Material: Intermediate 18 and furan-3-boronic acid.

MS (ES) MH⁺: 287.2 for C₁₆H₁₈N₂O₃.

### Intermediate 93

### 2-(3,5-Dimethylisoxazol-4-yl)-5-[(2R,6S)-2,6-dimethylmorpholin-4-yl]pyridine-4-carbaldehyde

Starting Material: Intermediate 18 and 3,5-dimethylisoxazole-4-boronic acid.

MS (ES) MH⁺: 316.2 for C₁₇H₂₁N₃O₃.

### Intermediate 94

### 5-[(2R,6S)-2,6-Dimethylmorpholin-4-yl]-2,4′-bipyridine-4-carbaldehyde

Starting Material: Intermediate 18 and pyridine-4-boronic acid.

MS (ES) MH⁺: 298.2 for C₁₇H₁₉N₃O₂.

### Intermediate 95

### 5-[(2R,6S)-2,6-Dimethylmorpholin-4-yl]-2-(1-methyl-1H-pyrazol-4-yl)pyridine-4-carbaldehyde

Starting Material: Intermediate 18 and 1-methyl-1H-pyrazol-4-boronic acid.

MS (ES) MH⁺: 298.2 for C₁₇H₁₉N₃O₂.

### Intermediate 96

### 6-(1-Benzofuran-2-yl)-3-[(2R,6S)-2,6-dimethylmorpholin-4-yl]pyridine-2-carbaldehyde

Starting Material: Intermediate 17 and 1-benzofuran-2-boronic acid.

MS (ES) MH⁺: 337.2 for C₂₀H₂₀N₂O₃.

### Intermediate 97

### 3-[(2R,6S)-2,6-Dimethylmorpholin-4-yl]-6-(1-methyl-1H-pyrazol-5-yl)pyridine-2-carbaldehyde

Starting Material: Intermediate 17 and 1-methyl-1H-pyrazole-5-boronic acid pinacol ester.

MS (ES) MH⁺: 301.2 for C₁₆H₂₀N₄O₂.

### Intermediate 98

### 5-[(2R,6S)-2,6-Dimethylmorpholin-4-yl]-4′-methoxy-2,3′-bipyridine-6-carbaldehyde

Starting Material: Intermediate 17 and 4-methoxy-pyridine-3-boronic acid.

MS (ES) MH⁺: 328.2 for C₁₈H₂₁N₃O₃.

### Intermediate 99

### 3-[(2R,6S)-2,6-Dimethylmorpholin-4-yl]-6-(1H-pyrazol-4-yl)pyridine-2-carbaldehyde

Starting Material: Intermediate 17 and 1H-pyrazole-4-boronic acid pinacol ester. MS (ES) MH⁺: 287.2 for C₁₅H₁₈N₄O₂.

### Intermediate 100

### 3-[(2R,6S)-2,6-Dimethylmorpholin-4-yl]-6-(pyrimidin-5-yl)pyridine-2-carbaldehyde

Starting Material: Intermediate 17 and pyrimidine-5-boronic acid.

MS (ES) MH⁺: 299.2 for C₁₆H₁₈N₄O₂.

### Intermediate 101

### 5-[(2R,6S)-2,6-Dimethylmorpholin-4-yl]-2,3′-bipyridine-6-carbaldehyde

Starting Material: Intermediate 17 and pyridine-3-boronic acid.

MS (ES) MH⁺: 298.4 for C₁₇H₁₉N₃O₂.

### Intermediate 102

### 3-[(2R,6S)-2,6-Dimethylmorpholin-4-yl]-6-(quinolin-8-yl)pyridine-2-carbaldehyde

Starting Material: Intermediate 17 and quinoline-8-boronic acid.

MS (ES) MH⁺: 348.0 for C₂₁H₂₁N₃O₂.

### Intermediate 103

### 6-(3,5-Dimethylisoxazol-4-yl)-3-[(2R,6S)-2,6-dimethylmorpholin-4-yl]pyridine-2-carbaldehyde

Starting Material: Intermediate 17 and 3,5-dimethylisoxazole-4-boronic acid.

MS (ES) MH⁺: 298.4 for C₁₇H₁₉N₃O₂.

### Intermediate 104

### 3-[(2R,6S)-2,6-Dimethylmorpholin-4-yl]-6-(furan-3-yl)pyridine-2-carbaldehyde

Starting Material: Intermediate 17 and 3-furan boronic acid.

MS (ES) MH⁺: 287.2 for C₁₆H₁₈N₂O₃.

### Intermediate 105

### 3-[(2R,6S)-2,6-Dimethylmorpholin-4-yl]-6-(1-methyl-1H-pyrazol-4-yl)pyridine-2-carbaldehyde

Starting Material: Intermediate 17 and 1-methylpyrazole-4-boronic acid pinacol ester.

MS (ES) MH⁺: 301.2 for C₁₆H₂₀N₄O₂.

### Intermediate 106

### 5-[(2R,6S)-2,6-Dimethylmorpholin-4-yl]-2,4′-bipyridine-6-carbaldehyde

Starting Material: Intermediate 24 and pyridine-4-boronic acid.

MS (ES) MH⁺: 298.4 for C₁₇H₁₉N₃O₂.

### Intermediate 107

### 2-[(2R,6S)-2,6-Dimethylmorpholin-4-yl]-5-(2-methylphenyl)pyridine-3-carbaldehyde

Starting Material: Intermediate 24 and 2-methylphenylboronic acid.

MS (ES) MH⁺: 311.1 for C₁₉H₂₂N₂O₂.

### Intermediate 108

### 2-[(2R,6S)-2,6-Dimethylmorpholin-4-yl]-5-(2-methoxyphenyl)pyridine-3-carbaldehyde

Starting Material: Intermediate 24 and 2-methoxyphenylboronic acid.

MS (ES) MH⁺: 327.1 for C₁₉H₂₂N₂O₃.

### Intermediate 109

### 2-[(2R,6S)-2,6-Dimethylmorpholin-4-yl]-5-phenylpyridine-3-carbaldehyde

Starting Material: Intermediate 24 and phenylboronic acid.

MS (ES) MH⁺: 311.1 for C₁₈H₂₀N₂O₂.

### Intermediate 110

### 5-[(2R,6S)-2,6-Dimethylmorpholin-4-yl]-2-(trimethylstannanyl)pyridine-4-carbaldehyde

A stirred suspension of the 2-bromo-5-[(2R,6S)-2,6-dimethylmorpholin-4-yl]pyridine-4-carbaldehyde (Intermediate 18, 300 mg, 1.0 mmol), and tetrakis(triphenylphosphine)palladium(0) (115 mg, 0.1 mmol) in dioxane (5 mL) was prepared. The reaction vessel was evacuated and degassed with argon 4 times. The mixture was then placed under an atmosphere of nitrogen and warmed to 100° C. Hexamethylditin (0.65 mL, 3.0 mmol) reagent was added by syringe during the warming period. The reaction mixture was stirred at 100° C. for 1.5 hours. The solvent was removed under vacuum and the residue was extracted with ethyl acetate and the organic phase was washed with water and brine and dried over anhydrous sodium sulphate. The organic layer was filtered and the filtrate was evaporated under vacuum to give the title compound as a light brown liquid. The stannanyl reagent thus obtained, was taken for the next step without further purification.

MS (ES) MH⁺: 384.2 for C₁₅H₂₄N₂O₂Sn.

### Intermediate 111

### 5-[(2R,6S)-2,6-Dimethylmorpholin-4-yl]-2-(quinolin-2-yl)pyridine-4-carbaldehyde

A stirred suspension of the 5-[(2R,6S)-2,6-dimethylmorpholin-4-yl]-2-(trimethylstannanyl)pyridine-4-carbaldehyde (Intermediate T110, 350 mg, 0.91 mmol), and 2-bromoquinoline (379 mg, 1.82 mmol) in DMF-Toloune (1:1, 6 mL) was prepared. The reaction vessel was evacuated and degassed with argon 4 times and filled with nitrogen. The mixture was then placed under dark conditions and was added tetrakis(triphenylphosphine)palladium(0) (105 mg, 0.091 mmol). The reaction mixture was stirred at 100° C. for 14 hours. The solvent was removed under vacuum and the residue was extracted with ethyl acetate and the organic phase was washed with water and brine and dried over anhydrous sodium sulphate. The organic layer was filtered and the filtrate was evaporated under vacuum and the residue was purified by silica gel column chromatography using pet. ether and ethyl acetate as eluent to give the title compound a as light brown liquid. Yield: 120 mg (38%).

MS (ES) MH⁺: 348.2 for C₂₁H₂₁N₃O₂.

### Intermediate 112

### 5-[(2R,6S)-2,6-Dimethylmorpholin-4-yl]-2-(2-methoxy-1,3-thiazol-4-yl)pyridine-4-carbaldehyde

The title compound was prepared from Intermediate 110 and 4-bromo-2-methoxy-1,3-thiazole using a procedure similar to the one described for the synthesis of Intermediate 111.

MS (ES) MH⁺: 334.2 for C₁₆H₁₉N₃O₃S.

### Intermediate 113

### 5-cyano-2-((2R,6S)-2,6-dimethylmorpholino)nicotinaldehyde

A 25 mL flask was charged with 5-bromo-2-((2S,6R)-2,6-dimethyl-morpholin-4-yl)-pyridine-3-carbaldehyde (Intermediate 24, 12.7 g, 42.4 mmols), DMAC (120 mL), K₄[Fe(CN)₆].3H₂O (3.94 g; 9.3 mmols; 0.22 equiv), sodium carbonate (4.5 g; 42.4 mmols; 1.0 equiv), and Pd(OAc)₂ (0.14 g, 0.6 mmol, 0.015 equiv). The flask was evacuated and filled with nitrogen (two times) and heated to 120° C. for 14 h, the reaction mixture was cooled to room temperature and diluted with 100 mL of EtOAc. The resulting slurry was filtered through Celite and the filtrate was concentrated. The product was isolated by washing the filtrate with water (3×75 ml) and brine (50 ml). The organic layer was dried over Na₂SO₄, and the volatiles were removed in vacuum and the residue was purified by column chromatography gave the product (4.5 g, 45%).

MS (ES) MH⁺: 246.

¹H NMR (400 MHz, DMSO) δ: 0.85 (d, 3H), 1.2 (d, 2H), 2.8 (t, 2H), 3.6 (m, 2H), 4.0 (d, 2H), 8.5 (d, 1H), 8.65 (d, 1H), 9.75 (s, 1H).

### Intermediate 114

### 2-[(2R,6S)-2,6-Dimethylmorpholin-4-yl]-5-(1,3,4-thiadiazol-2-yl)pyridine-3-carbaldehyde

To a stirred and degassed solution of 2-((2S,6R)-2,6-dimethyl-morpholin-4-yl)-5-(4,4,5,5-tetramethyl[1,3,2]dioxaborolan-2-yl)-pyridine-3-carbaldehyde (Intermediate 36) (250 mg, 0.61 mmol) in 10 ml acetonitrile:water (4:1), was added sodium carbonate (65 mg, 0.61 mmol), 2-bromo-1,3,4 thiadiazole (120 mg, 0.73 mmol), 2-dicyclohexylphosphino 2′,4′6′-triisopropylbiphenyl (X-phos) (88 mg, 0.18 mmol) and tris(dibenzylideneacetone)dipalladium(0) (56 mg, 0.061 mmol), sequentially and the reaction mixture was heated to 90° C. for 12 hours. The reaction mixture was cooled to room temperature and concentrated. The residue thus obtained was purified over silica gel column using a gradient of ethyl acetate in pet. ether to give product as yellow solid. Yield: 230 mg.

MS (ES) MH⁺: 305.2 for C₁₄H₁₆N₄O₂S.

Intermediates 115 to 133 were prepared from the indicated starting materials using a procedure similar to the one described for the synthesis of Intermediate 114.

### Intermediate 115

### 2-[(2R,6S)-2,6-Dimethylmorpholin-4-yl]-5-(5-methyl-1,3,4-thiadiazol-2-yl)pyridine-3-carbaldehyde

Starting Material: Intermediate 36 and 2-bromo-5-methyl-1,3,4-thiadiazole.

MS (ES) MH⁺: 319.4 for C₁₅H₁₈N₄O₂S.

### Intermediate 116

### 2-[(2R,6S)-2,6-Dimethylmorpholin-4-yl]-5-(1,3-thiazol-2-yl)pyridine-3-carbaldehyde

Starting Material: Intermediate 36 and 2-bromothiazole.

MS (ES) MH⁺: 304.2 for C₁₅H₁₇N₃O₂S.

### Intermediate 117

### 2-[(2R,6S)-2,6-Dimethylmorpholin-4-yl]-5-(1,3-thiazol-5-yl)pyridine-3-carbaldehyde

Starting Material: Intermediate 36 and 4-bromothiazole.

MS (ES) MH⁺: 304.2 for C₁₅H₁₇N₃O₂S.

### Intermediate 118

### 2-[(2R,6S)-2,6-Dimethylmorpholin-4-yl]-5-(thiophen-2-yl)pyridine-3-carbaldehyde

Starting Material: Intermediate 36 and 2-bromothiophene.

MS (ES) MH⁺: 303.2 for C₁₆H₁₈N₂O₂S.

### Intermediate 119

### 5-(1-Benzothiophen-2-yl)-2-[(2R,6S)-2,6-dimethylmorpholin-4-yl]pyridine-3-carbaldehyde

Starting Material: Intermediate 36 and 2-bromobenzthiophene.

MS (ES) MH⁺: 353.4 for C₂₀H₂₀N₂O₂S.

### Intermediate 120

### 2-[(2R,6S)-2,6-Dimethylmorpholin-4-yl]-5-(5-methylthiophen-2-yl)pyridine-3-carbaldehyde

Starting Material: Intermediate 36 and 2-bromo-5-methylthiophene.

MS (ES) MH⁺: 317.4 for C₁₇H₂₀N₂O₂S.

### Intermediate 121

### 2-[(2R,6S)-2,6-Dimethylmorpholin-4-yl]-5-(3-methylthiophen-2-yl)pyridine-3-carbaldehyde

Starting Material: Intermediate 36 and 2-methylthiophene.

MS (ES) MH⁺: 317.2 for C₁₇H₂₀N₂O₂S.

### Intermediate 122

### 2-[(2R,6S)-2,6-Dimethylmorpholin-4-yl]-5-[5-(1H-tetrazol-5-yl)thiophen-2-yl]pyridine-3-carbaldehyde

Starting Material: Intermediate 36 and 5-(5-bromo-2-thienyl)-2H-tetrazole.

MS (ES) MH⁺: 371.4 for C₁₇H₁₈N₆O₂S.

### Intermediate 123

### 2-[(2R,6S)-2,6-Dimethylmorpholin-4-yl]-5-(1-methyl-1H-imidazol-2-yl)pyridine-3-carbaldehyde

Starting Material: Intermediate 36 and 2-bromo-1-methylimidazole.

MS (ES) MH⁺: 301.4 for C₁₆H₂₀N₄O₂.

### Intermediate 124

### 2-[(2R,6S)-2,6-Dimethylmorpholin-4-yl]-5-(1-methyl-1H-imidazol-4-yl)pyridine-3-carbaldehyde

Starting Material: Intermediate 36 and 4-bromo-1-methylimidazole.

MS (ES) MH⁺: 301.4 for C₁₆H₂₀N₄O₂.

### Intermediate 125

### 2-[(2R,6S)-2,6-Dimethylmorpholin-4-yl]-5-(4-methylthiophen-3-yl)pyridine-3-carbaldehyde

Starting Material: Intermediate 36 and 3-bromo-4-methylthiophene.

MS (ESP) MH⁺: 317.2 for C₁₇H₂₀N₂O₂S.

### Intermediate 126

### 2-[(2R,6S)-2,6-Dimethylmorpholin-4-yl]-5-(1H-imidazol-2-yl)pyridine-3-carbaldehyde

Starting Material: Intermediate 36 and 2-bromoimidazole.

MS (ESP) MH⁺: 287.4 for C₁₅H₁₈N₄O₂.

### Intermediate 127

### 2-[(2R,6S)-2,6-Dimethylmorpholin-4-yl]-5-[5-(1H-pyrazol-5-yl)thiophen-2-yl]pyridine-3-carbaldehyde

Starting Material: Intermediate 36 and 5-bromopyrazole.

MS (ESP) MH⁺: 368.4 for C₁₉H₂₀N₄O₂S.

### Intermediate 128

### 2-[(2R,6S)-2,6-Dimethylmorpholin-4-yl]-5-(1H-imidazol-4-yl)pyridine-3-carbaldehyde

Starting Material: Intermediate 36 and 4-bromoimidazole.

MS (ESP) MH⁺: 286.4 for C₁₅H₁₈N₄O₂.

### Intermediate 129

### 6′-[(2R,6S)-2,6-Dimethylmorpholin-4-yl]-2,3′-bipyridine-5′-carbaldehyde

Starting Material: Intermediate 36 and 2-bromopyridine.

MS (ES) MH⁺: 298.2 for C₁₇H₁₉N₃O₂.

### Intermediate 130

### 2-[(2R,6S)-2,6-Dimethylmorpholin-4-yl]-5-(quinolin-2-yl)pyridine-3-carbaldehyde

Starting Material: Intermediate 36 and 2-bromoquinoline.

MS (ES) MH⁺: 348.4 for C₂₁N₂₁N₃O₂

### Intermediate 131

### 5-(1H-Benzimidazol-2-yl)-2-[(2R,6S)-2,6-dimethylmorpholin-4-yl]pyridine-3-carbaldehyde

Starting Material: Intermediate 36 and 2-bromo-1H-benzimidazole.

MS (ES) MH⁺: 337.4 for C₁₉H₂₀N₄O₂.

### Intermediate 132

### 5-[4-(Dimethylamino)phenyl]-2-[(2R,6S)-2,6-dimethylmorpholin-4-yl]pyridine-3-carbaldehyde

Starting Material: Intermediate 36 and 4-bromo-N,N-dimethylaniline.

MS (ES) MH⁺: 340.2 for C₂₀H₂₅N₃O₂.

### Intermediate 133

### 2-[(2R,6S)-2,6-Dimethylmorpholin-4-yl]-5-(2,4-dimethyl-1,3-thiazol-5-yl)pyridine-3-carbaldehyde

Starting Material: Intermediate 36 and 5-bromo-2,4-dimethyl-1,3-thiazole.

MS (ES) MH⁺: 332.2 for C₁₇H₂₁N₃O₂S.

### Intermediate 134

### (2,6-Difluoro-5-(5-methyl-1,3,4-thiadiazol-2-yl)pyridin-3-yl)methanol

Acetyl chloride (6 mL) was added dropwise to MeOH (40 mL) and the HCl solution obtained was cooled to 20° C. A solution of 2-(5-((tert-butyldiphenylsilyloxy)methyl)-2,6-difluoropyridin-3-yl)-5-methyl-1,3,4-thiadiazole (Intermediate 137, 1.1 g, 2.3 mmol) in Et2O (10 mL) was added slowly. The reaction mixture was allowed to warm to room temperature. LC/MS after 30 minutes indicates formation of product and LC/MS after 3 hours indicates reaction is complete. The reaction mixture was diluted with water and extracted three times with ethyl acetate. The combined organic extracts were dried over magnesium sulfate, filtered and evaporated to a yellow solid. Isco column (0%-100% ethyl acetate/dichloromethane) afforded the desired compound as a light yellow solid (0.46 g, 81% yield).

MS (ES) MH⁺: 244 for C₉H₇F₂N₃OS.

¹H NMR: 2.8 (s, 3H), 4.6 (d, 2H), 5.7 (t, 1H), 8.9 (t, 1H).

Intermediates 135 and 136 were prepared from the indicated starting materials using a procedure similar to the one described for the synthesis of Intermediate 134.

### Intermediate 135

### (2,6-Difluoro-5-iodopyridin-3-yl)methanol

Starting Material: Intermediate 13.

MS (ES) MH⁺: 272 for C₆H₄F₂₁NO.

¹H NMR: 4.5 (d, 2H), 5.5 (t, 1H), 8.5 (t, 1H).

### Intermediate 136

### (6-Bromo-4-chloro-3-((2R,6S)-2,6-dimethylmornholino)-5-fluoropyridin-2-yl)methanol

Starting Material: Intermediate 14.

MS (ES) MH⁺: 355 for C₁₂H₁₅BrClFN₂O₂.

¹H NMR (DMSO-d⁶): 1.1 (d, 6H), 2.9 (m, 4H), 3.7-3.8 (m, 2H), 4.6 (d, 2H), 5.3 (s, broad, 1H).

### Intermediate 137

### 2-(5-((tert-Butyldiphenylsilyloxy)methyl)-2,6-difluoropyridin-3-yl)-5-methyl-1,3,4-thiadiazole

To a solution of N′-acetyl-5-((tert-butyldiphenylsilyloxy)methyl)-2,6-difluoronicotinohydrazide (Intermediate 138, 6.12 g, 12.7 mmol) and phosphorus pentasulfide (2.81 g, 12.7 mmol) in toluene (100 mL) was added hexamethyldisiloxane (4.30 mL, 20.2 mmol) and the reaction was heated to reflux. LC/MS after 2 hours indicates reaction was complete. The reaction mixture was allowed to cooled to room temperature and was diluted with acetone (30 mL). K₂CO₃ (5.97 mL, 31.6 mmol, Fisher) was added carefully and the reaction mixture was concentrated to a red oil. The reaction mixture was diluted with water and extracted three times with dichloromethane. The combined organic extracts were dried over magnesium sulfate, filtered and evaporated to yield an orange oil. Isco column (50%-100% dichloromethane/hexane) afforded the desired compound as a yellow oil (1.98 g, 33% yield).

MS (ES) MH⁺: 482 for C₂₅H₂₅F₂N₃OSSi.

¹H NMR: 1.0 (s, 9H), 2.8 (s, 3H), 4.9 (s, 2H), 7.5 (m, 6H), 7.6 (m, 4H), 8.9 (t, 1H).

### Intermediate 138

### N′-Acetyl-5-((tert-butyldiphenylsilyloxy)methyl)-2,6-difluoronicotinohydrazide

5-((tert-Butyldiphenylsilyloxy)methyl)-2,6-difluoronicotinic acid (Intermediate 139, 7.72 g, 18.1 mmol) was suspended in SOCl₂ (50 mL) and heated to reflux. The reaction was stirred for 2 hours. The solution was cooled to room temperature and concentrated. The residue was suspended in methylene chloride and concentrated. The residue was suspended in CH₂Cl₂ (50 mL) and acetic acid hydrazide (1.67 g, 22.6 mmol) and DIEA (3.9 mL, 22.6 mmol) were added. After stirring for 3 hours, the reaction was quenched with saturated ammonium chloride and extracted with dichloromethane. The combined organic extracts were dried over magnesium sulfate, filtered and evaporated to a brown oil (6.12 g, 70%).

MS (ES) MH⁺: 484 for C₂₅H₂₇F₂N₃O₃Si

### Intermediate 139

### 5-((tert-Butyldiphenylsilyloxy)methyl)-2,6-difluoronicotinic acid

A solution of n-butyllithium, 2.5 M in hexanes (11.3 mL, 28.2 mmol) was added slowly to a solution of diisopropylamine (7.4 mL, 51.6 mmol) in THF (50 mL) cooled to below −70° C. The solution was warmed to −10° C. and recooled to below −70° C. A solution of 3-((tert-butyldiphenylsilyloxy)methyl)-2,6-difluoropyridine (Intermediate 140, 9.0 g, 23.5 mmol) in THF (4 mL) was added slowly keeping the temperature below −60° C. The mixture was stirred at −70° C. or below for 2 hours before excess CO2 (dry ice) was slowly bubbled through the reaction. After stirring for 15 min at −70° C. or below the mixture was warmed to room temperature. The reaction mixture was diluted with water and washed twice with ethyl acetate. The organic extracts were dried over magnesium sulfate, filtered and evaporated to yield an orange oil (9.67 g, 96% yield).

MS (ES) MH⁺: 428 for C₂₃H₂₃F₂NO₃Si

### Intermediate 140

### 3-((tert-Butyldiphenylsilyloxy)methyl)-2,6-difluoropyridine

(2,6-Difluoropyridin-3-yl)methanol (Intermediate 4, 1.5 g, 10.2 mmol) and imidazole (0.73 g, 10.7 mmol) were combined in dichloromethane (50 mL) and cooled to 0° C. t-butyldiphenylchlorosilane (2.8 mL, 10.7 mmol) was added dropwise so the temperature never rose above 10° C. The reaction mixture was allowed to warm to room temperature and for 1 hour. LC/MS indicates reaction is complete. The reaction mixture was diluted with dichloromethane and poured into 1N HCl (150 mL) and the layers were separated. The organic portion was washed with sat NaHCO₃. The organic was dried with MgSO₄ and concentrated to a colorless oil (3.92 g, 100% yield).

MS (ES) MH⁺: 384 for C₂₂H₂₃F₂NOSi.

¹H NMR: 1.0 (s, 9H), 4.75 (s, 2H), 7.2 (d, 1H), 7.5 (m, 6H), 7.6 (m, 4H), 8.2 (t, 1H).

### Intermediate 141

### (2R,6S)-4-(2-((tert-Butyldiphenylsilyloxy)methyl)-5-fluoropyridin-3-yl)-2,6-dimethylmorpholine

The title compound was prepared from Intermediate 6 using a procedure similar to the one described for the synthesis of Intermediate 140.

MS (ES) MH⁺: 479 for C₂₈H₃₅FNO₂Si.

¹H NMR (CDCl₃): δ 1.0 (s, 9H), 1.15 (d, 6H, 2.4 (t, 2H), 3.0 (d, 2H), 3.6-3.7 (m, 2H), 4.8 (s, 2H), 7.0 (d, 1H), 7.4 (m, 6H), 7.7 (m, 4H), 8.2 (s, 1H).

### Intermediate 142

### 2-((2R,6S)-2,6-Dimethylmorpholino)-6-fluoronicotinaldehyde

To a solution of (2-((2R6S)-2,6-dimethylmorpholino)-6-fluoropyridin-3-yl)methanol (Intermediate 146, 0.22 g, 0.9 mmol) and NMO (0.16 g, 1.4 mmol) in 1:1 acetonitrile (5 mL)/dichloromethane (5 mL) was added TPAP (0.032 g, 0.09 mmol) and the reaction was stirred at room temperature. LC/MS after 15 minutes indicates conversion to product. The reaction mixture was filtered through plug of silica gel and washed through with EtOAc. The filtrate was concentrated to a yellow oil (0.16 g, 72%).

MS (ES) MH⁺: 239 for C₁₂H₁₅FN₂O₂.

¹H NMR: 1.1 (d, 6H), 2.7 (dd, 2H), 3.7 (m, 4H), 6.6 (d, 1H), 8.3 (t, 1H), 9.8 (s, 1H).

Intermediates 143 to 145 were prepared from the indicated starting materials using a procedure similar to the one described for the synthesis of Intermediate 142.

### Intermediate 143

### 2-((2R,6S)-2,6-Dimethylmorpholino)-6-fluoro-5-(pyridin-2-ylethynyl)nicotinaldehyde

Starting Material: Intermediate 148.

MS (ES) MH⁺: 340 for C₁₉H₁₈FN₃O₂.

¹H NMR: 1.1 (d, 6H), 1.8 (m, 3H), 2.8 (dd, 2H), 3.7 (m, 2H), 3.9 (d, 2H), 7.4 (dd, 1H), 7.6 (d, 1H), 7.9 (t, 1H), 8.5 (d, 1H), 8.6 (m, 1H), 9.8 (s, 1H).

### Intermediate 144

### 2-((2R,6S)-2,6-Dimethylmorpholino)-6-fluoro-5-(pyrazin-2-ylethynyl)nicotinaldehyde

Starting Material: Intermediate 149.

MS (ES) MH⁺: 341 for C₁₈H₁₇FN₄O₂.

¹H NMR: 1.1 (d, 6H), 2.8 (dd, 2H), 3.7 (m, 2H), 3.9 (d, 2H), 8.6 (d, 1H), 8.7 (dd, 2H), 8.9 (s, 1H), 9.8 (s, 1H).

### Intermediate 145

### 2-((2R,6S)-2,6-Dimethylmorpholino)-6-fluoro-5-((4-methoxyphenyl)ethynyl)nicotinaldehyde

Starting Material: Intermediate 150.

MS (ES) MH⁺: 369 for C₂₁H₂₁FN₂O₃.

¹H NMR: 1.1 (d, 6H), 2.8 (dd, 2H), 3.7 (m, 3H), 3.8 (s, 3H), 3.9 (d, 1H), 7.0 (d, 1H), 7.5 (d, 2H), 8.4 (d, 1H), 9.8 (s, 1H).

### Intermediate 146

### (2-((2R,6S)-2,6-dimethylmorpholino)-6-fluoropyridin-3-yl)methanol

To a solution of 2-((2R,6S)-2,6-dimethylmorpholino)-6-fluoronicotinic acid (1.1 g, 4.4 mmol, Intermediate 147) in THF (75 mL) at 0° C. was added NaBH₄ (0.60 g, 15.9 mmol) portionwise to reduce foaming. A solution of I₂ (1.9 g, 7.5 mmol, Fisher) in THF (75 mL) was then added dropwise so the temperature in the reaction did not rise above 10° C. The reaction mixture was heated at reflux overnight. After cooling to room temperature, MeOH (100 mL) was added slowly. Gas evolution was observed. The reaction mixture was concentrated to a yellow solid which was suspended in 1N NaOH (100 mL) and stirred at room temperature for 1 hr. The reaction mixture was diluted with ethyl acetate and extracted three times with ethyl acetate. The combined organic extracts were dried over magnesium sulfate, filtered and evaporated to yield a yellow oil. Isco column (0%-50% ethyl acetate/dichloromethane) afforded the desired compound as a yellow oil (0.22 g, 21% yield).

MS (ES) MH⁺: 241 for C₁₂H₁₇FN₂O₂.

¹H NMR: 1.1 (d, 6H), 2.4 (dd, 2H), 3.4 (d, 2H), 3.7 (m, 2H), 4.4 (d, 2H), 5.3 (t, 1H), 6.6 (d, 1H), 7.9 (t, 1H).

### Intermediate 147

### 2-((2R,6S)-2,6-Dimethylmorpholino)-6-fluoronicotinic acid

To a solution of 2,6-difluoronicotinic acid (1.0 g, 6.3 mmol) in THF (20 mL) at −78° C. was added LiHMDS, 0.9M in methylcyclohexane (7.7 mL, 6.9 mmol) and the reaction was stirred at −78° C. for 1 hour. In a separate flask, a solution of (2R,6S)-2,6-dimethylmorpholine (0.78 mL, 6.3 mmol) in THF (20 mL) was cooled to −78° C. LiHMDS, 0.9M in methylcyclohexane (7.7 mL, 6.9 mmol) was added and the reaction was stirred for 1 hour. The contents of the second flask were added slowly to the first flask, maintaining the temperature at −78° C. LC/MS after stirring overnight indicates reaction is complete. The reaction mixture was diluted with 1N HCl and extracted three times with ethyl acetate. The combined organic extracts were dried over magnesium sulfate, filtered and evaporated to a yellow solid (1.3 g, 83% yield).

MS (ES) MH⁺: 255 for C₁₂H₁₅FN₂O₃

¹H NMR: 1.1 (d, 6H), 2.6 (dd, 2H), 3.6 (m, 4H), 6.4 (d, 1H), 8.1 (t, 1H).

### Intermediate 148

### (2-((2R,6S)-2,6-dimethylmorpholino)-6-fluoro-5-(pyridin-2-ylethynyl)pyridin-3-yl)methanol

(2-((2R,6S)-2,6-Dimethylmorpholino)-6-fluoro-5-iodopyridin-3-yl)methanol (Intermediate 151, 0.094 g, 0.26 mmol), 2-ethynylpyridine (0.029 g, 0.28 mmol), CuI (2.445 mg, 0.01 mmol), Et3N (0.29 mL, 2.1 mmol), and dichlorobis(triphenylphosphine)palladium(II) (0.18 g, 0.26 mmol) were combined in anh. acetonitrile (5 mL) and heated to reflux. The reaction was stirred overnight. LC/MS indicates reaction is complete. The reaction was cooled to room temperature and filtered through celite. The filtrate was concentrated to a brown oil. Isco column (0%-100% ethyl acetate/dichloromethane) afforded the desired compound as a brown solid (0.046 g, 53% yield).

MS (ES) MH⁺: 342 for C₁₉H₂₀FN₃O₂.

¹H NMR: 1.1 (d, 6H), 2.6 (dd, 2H), 3.7 (d, 4H), 4.4 (d, 2H), 5.5 (t, 1H), 7.4 (dd, 1H), 7.6 (d, 1H), 7.9 (dd, 1H), 8.0 (d, 1H), 8.6 (s, broad, 1H).

Intermediates 149 and 150 were prepared from the indicated starting materials using a procedure similar to the one described for the synthesis of Intermediate 148.

### Intermediate 149

### (2-((2R,6S)-2,6-Dimethylmorpholino)-6-fluoro-5-(pyrazin-2-ylethynyl)pyridin-3-yl)methanol

Starting Material: Intermediate 151 and 2-ethynylpyrazine.

MS (ES) MH⁺: 343 for C₁₈H₁₉FN₄O₂

¹H NMR: 1.1 (d, 6H), 2.6 (dd, 2H), 3.7 (m, 4H), 4.4 (d, 2H), 5.5 (t, 1H), 8.0 (d, 1H), 8.6 (dd, 1H), 8.9 (s, 1H).

### Intermediate 150

### (2-((2R,6S)-2,6-Dimethylmornholino)-6-fluoro-5-((4-methoxyphenyl)ethynyl)pyridin-3-yl)methanol

Starting Material: Intermediate 151 and 1-ethynyl-4-methoxybenzene.

MS (ES) MH⁺: 371 for C₂₁H₂₃FN₂O₃

### Intermediate 151

### (2-((2R,6S)-2,6-Dimethylmorpholino)-6-fluoro-5-iodopyridin-3-yl)methanol

To a solution of 2-((2R,6S)-2,6-dimethylmorpholino)-6-fluoro-5-iodonicotinic acid (Intermediate 152, 1.5 g, 3.9 mmol) in THF (25 mL) at 0° C. was added BH₃ in THF, 1M (11.6 mL, 11.6 mmol) slowly. The reaction mixture was allowed to warm to room temperature and stir. After 1 hour of gentle heating, LC/MS indicates reaction is complete. The reaction mixture was diluted with the dropwise addition of water and extracted three times with ethyl acetate. The combined organic extracts were dried over magnesium sulfate, filtered and evaporated (1.15 g, 81% yield).

MS (ES) MH⁺: 367 for C₁₂H₁₆FIN₂O₂.

¹H NMR: 1.1 (d, 6H), 2.4 (dd, 2H), 3.4 (d, 2H), 3.7 (dd, 2H), 4.4 (d, 2H), 5.4 (t, 1H), 8.1 (d, 1H).

### Intermediate 152

### 2-((2R,6S)-2,6-Dimethylmorpholino)-6-fluoro-5-iodonicotinic acid

To a solution of 2,6-difluoro-5-iodonicotinic acid (Intermediate 153, 0.24 g, 0.84 mmol) in acetonitrile (5 mL) at 0° C. was added DIEA (0.15 mL, 0.84 mmol) followed by (2R,6S)-2,6-dimethylmorpholine (0.10 mL, 0.84 mmol) and the reaction was stirred at room temperature. LC/MS after 15 minutes indicates the reaction is complete. The reaction mixture was diluted with water and 1N HCl and extracted three times with ethyl acetate. The combined organic extracts were dried over magnesium sulfate, filtered and evaporated (0.20, 63% yield).

MS (ES) MH⁺: 381 for C₁₂H₁₄FIN₂O₃.

¹H NMR: 1.1 (d, 6H), 2.6 (dd, 2H), 3.6 (m, 2H), 3.7 (d, 2H), 8.2 (d, 1H).

### Intermediate 153

### 2,6-Difluoro-5-iodonicotinic acid

To a solution of (2,6-difluoro-5-iodopyridin-3-yl)methanol (Intermediate 135, 0.25 g, 0.90 mmol) in acetone (10 mL) at 0° C. was added a solution of CrO₃ (0.18 g, 1.8 mmol) in H₂SO₄ (1 ml,) and H2O (4 ml) and the reaction was warmed to room temperature. LC/MS after 1 hour indicates reaction is complete. The reaction mixture was diluted with water and extracted three times with dichloromethane. The combined organic extracts were dried over magnesium sulfate, filtered and evaporated to yield a white solid (0.24 g, 94% yield).

MS (ES) M-H⁻: 284 for C₆H₂F₂INO₂.

¹H NMR: 8.8 (t, 1H), 13.9 (s, 1H).

### Intermediate 154

### 5-[(1,3-di-tert-Butyl-2,4,6-trioxotetrahydropyrimidin-5(2H)-ylidene)methyl]-6-[(2R,6S)-2,6-dimethylmorpholin-4-yl]pyridine-3-carbonitrile

A solution of 5-cyano-2-((2R,6S)-2,6-dimethylmorpholino)nicotinaldehyde (Intermediate 113, 2 g, 6.6 mmol) and di-t-butylbarbituric acid (1.76 g, 7.3 mmol) in dry IPA (30 mL) was heated at 95° C. overnight, under N₂. The reaction mixture cooled to room temperature and filtered. The solid thus obtained was recrystallized with ethyl acetate-hexane to give product as a yellow solid. (Yield: 2.1 g, 55%).

MS (ES) MH⁺: 468.

¹H NMR (400 MHz) δ: 1.9-1.2 (m, 6H), 1.6 (s, 18H), 2.8 (t, 2H), 3.75-3.8 (m, 4H), 3.6 (m, 2H), 7.9 (s, 1H), 8.15 (s, 1H), 8.4 (s, 1H).

### Intermediate 155

### 1,3-di-tert-Butyl-5-({2-[(2R,6S)-2,6-dimethylmorpholin-4-yl]-5-(2H-tetrazol-5-yl)pyridin-3-yl}methylidene)pyrimidine-2,4,6(1H,3H,5H)-trione

A mixture of 5-[(1,3-di-tert-butyl-2,4,6-trioxotetrahydropyrimidin-5(2H)-ylidene)methyl]-6-[(2R,6S)-2,6-dimethylmorpholin-4-yl]pyridine-3-carbonitrile (Intermediate 154, 0.2 g, 0.42 mmol) and azidotributyltin (0.28 g, 0.84 mmol) in toluene (5 ml) was refluxed for 14 hours. The toluene was removed under vacuum and the residue was dissolved in ethyl acetate (15 ml) and washed with water (4×10 ml) and brine. The organic phase was concentrated and the residue was purified by column chromatography on silica gel (100% pet. ether gradient to 50% ethyl acetate) to give product (100 mg, 44%).

MS (ES) MH⁺: 511 (MH).

### Example 1

### (6aS,7S,9R)-rel-7,9-Dimethyl-3-(1,3,4-thiadiazol-2-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

A solution of 2-[(2R,6S)-2,6-dimethyl morpholin-4-yl]-5-(1,3,4-thiadiazol-2-yl)pyridine-3-carbaldehyde (Intermediate 114, 32 mg, 0.0107 mmol) and barbituric acid (16.4 mg, 0.0128 mmol) in anhydrous IPA (2 mL) was heated to 90° C. for 12 hours. The reaction mixture was cooled to room temperature and concentrated. The residue thus obtained was purified over neutral alumina column, using a gradient of ethyl acetate in pet ether to give product as solid. MS (ES) MH⁺: 415.0 for C₁₈H₁₈N₆O₄S; ¹H NMR (400 MHz, DMSO): δ 1.0 (d, 3H), 1.2 (d, 3H), 2.8 (m, 1H), 2.9 (d, 1H), 3.6 (m, 3H), 4.0 (d, 1H), 5.1 (dd, 1H), 7.8 (s, 1H), 8.6 (d, 1H), 9.5 (s, 1H), 11.7 (s, broad, 2H).

Examples 2 to 113 were prepared from pyrimidine-2,4,6(1H,3H,5H)-trione and the indicated starting material using a procedure similar to the one described for the synthesis of Example 1.

### Example 2

### 3-Bromo-7,9-Dimethyl-5,6a,7,8,9,10-hexahydro-1′H-spiro[pyrido[1,2-a][1,7]naphthyridine-6,5′-pyrimidine]-2′,4′,6′(3′H)-trione

Starting Material: Intermediate 19.

MS (ES) MH⁺: 407 for C₁₇H₁₉BrN₄O₃.

¹H NMR (300 MHz, DMSO): 0.7 (2d, 3H), 0.9 (2d, 3H, 4:1 ratio), 1.0 (q, 1H), 1.4-1.8 (m, 3H), 2.7 (m, 1H), 2.8 and 2.9 (2d, 1H, 4:1 ratio), 3.2 (m, 1H), 3.4 (m, 1H), 3.6 (m, 1H), 3.9-4.1 (m, 1H), 7.1 (2s, 1H, 4:1 ratio), 7.9 (2s, 1H, 4:1 ratio), 11.5 (2s, 1H, 4:1 ratio), 11.7 (2s, 1H, 4:1 ratio).

### Example 3

### (6aS,7S,9R)-rel-3-Bromo-7,9-dimethyl-6a,7,9,10-tetrahydro-1′H,5H-spiro[[1,4]oxazino[4,3-a][1,7]naphthyridine-6,5′-pyrimidine]-2′,4′,6′(3′H)-trione

Starting Material: Intermediate 18.

MS (ES) MH⁺: 409 for C₁₆H₁₇BrN₄O₄.

¹H NMR (300 MHz, DMSO): 0.95 (d, 3H), 1.1 (d, 3H), 2.7-3.0 (m, 2H), 3.4-3.7 (m, 4H), 4.1 (d, 1H), 7.1 (s, 1H), 8.0 (s, 1H), 11.6 (s, 1H), 11.8 (s, 1H).

### Example 4

### 1-chloro-5,6a,7,8,9,10-Hexahydro-1′H-spiro[pyrido[1,2-a][1,7]naphthyridine-6,5′-pyrimidine]-2′,4′,6′(3′H)-trione

Starting Material: Intermediate 29.

MS (ES) MH⁺: 335 for C₁₅H₁₅ClN₄O₃.

¹H NMR: 1.27-1.69 (m, 6H), 3.05 (m, 1H), 3.24 (d, 3H), 3.78 (m, 1H), 7.05 (d, 1H), 7.73 (d, 1H), 11.36 (s, 1H), 11.59 (s, 1H).

### Example 5

### 4-Fluoro-5,6a,7,8,9,10-Hexahydro-1′H-spiro[pyrido[1,2-a][1,7]naphthyridine-6,5′-pyrimidine]-2′,4′,6′(3′H)-trione

Starting Material: Intermediate 21.

MS (ES) MH⁺: 319 for C₁₅H₁₅FN₄O₃.

¹H NMR (300 MHz, DMSO): 1.05 (m, 1H), 1.4 (m, 3H), 1.6 (m, 1H), 1.7 (m, 1H), 3.0 (t, 1H), 3.5 (d, 1H), 4.1 (m, 1H), 7.8 (s, 1H), 8.1 (s, 1H), 11.2 (s, 1H), 11.3 (s, 1H).

### Example 6

### (6aS,7S,9R)-rel-4-Fluoro-7,9-dimethyl-6a,7,9,10-tetrahydro-1′H,5H-spiro[[1,4]oxazino[4,3-a][1,7]naphthyridine-6,5′-pyrimidine]-2′,4′,6′(3′H)-trione

Starting Material: Intermediate 20.

MS (ES) MH⁺: 349 for C₁₆H₁₇FN₄O₄.

¹H NMR (300 MHz, DMSO): 1.0 (d, 3H), 1.1 (d, 3H), 2.8 (t, 2H), 3.5 (m, 3H), 3.6 (m, 1H), 4.1 (d, 1H), 7.8 (s, 1H), 8.1 (s, 1H), 11.55 (s, 1H), 11.8 (s, 1H).

### Example 7

### (6aR,7R)-7,9-Dimethyl-5,6a,7,8,9,10-hexahydro-1′H-spiro[pyrido[1,2-a][1,8]naphthyridine-6,5′-pyrimidine]-2′,4′,6′(3′H)-trione

Starting Material: Intermediate 22.

MS (ES) MH⁺: 329 for C₁₇H₂₀N₄O₃.

¹H NMR (300 MHz, DMSO): 0.7 (d, 3H), 0.9 (d, 3H), 1.7 (m, 3H), 2.7 (m, 1H), 3.4 (m, 1H), 3.7 (d, 1H), 4.1 (d, 1H), 4.9 (m, 1H), 6.5 (m, 1H), 7.15 (m, 1H), 7.9 (s, 1H), 11.5 (s, 1H), 11.7 (s, 1H).

### Example 8

### (6aR,7R)-3-Bromo-7,9-dimethyl-5,6a,7,8,9,10-hexahydro-1′H-spiro[pyrido[1,2-a][1,5]naphthyridine-6,5′-pyrimidine]-2′,4′,6′(3′H)-trione

Starting Material: Intermediate 23.

MS (ES) MH⁺: 408 for C₁₇H₁₉BrN₄O₃.

¹H NMR (300 MHz, DMSO): 0.7 (d, 3H), 0.9 (d, 3H), 1.6 (m, 3H), 2.7 (m, 1H), 3.0 (d, 1H), 3.2 (d, 2H), 3.55 (d, 1H), 3.8 (d, 1H), 4.3 (s, 1H), 7.15 (q, 2H), 11.5 (s, 1H), 11.6 (s, 1H).

### Example 9

### (6aS,7S,9R)-rel-3-Bromo-7,9-dimethyl-6a,7,9,10-tetrahydro-1′H,5H-spiro[[1,4]oxazino[4,3-a][1,8]naphthyridine-6,5′-pyrimidine]-2′,4′,6′(3′H)-trione

Starting Material: Intermediate 24.

MS (ES) MH⁺: 409 for C₁₆H₁₇BrN₄O₄.

¹H NMR (300 MHz, DMSO): 0.9 (d, 3H), 1.1 (d, 3H), 2.7 (t, 1H), 2.9 (d, 1H), 3.4 (m, 2H), 3.46 (m, 2H), 3.8 (d, 1H), 4.9 (d, 1H), 7.35 (s, 1H), 8.0 (s, 1H), 11.6 (s, 1H), 11.9 (s, 1H).

### Example 10

### 3-Bromo-5,6a,7,8,9,10-hexahydro-1′H-spiro[pyrido[1,2-a][1,8]naphthyridine-6,5′-pyrimidine]-2′,4′,6′(3′H)-trione

Starting Material: Intermediate 25.

MS (ES) MH⁺: 379 for C₁₅H₁₅BrN₄O₃.

¹H NMR (300 MHz, DMSO): 1.2 (m, 2H), 1.5 (m, 3H), 1.7 (m, 1H), 2.7 (t, 1H), 2.95 (d, 1H), 3.2 (m, 1H), 3.6 (d, 1H), 4.7 (d, 1H), 7.5 (s, 1H), 8.0 (s, 1H), 11.3 (s, 1H), 11.3 (s, 1H).

### Example 11

### (6aR,7R)-3-Bromo-7,9-dimethyl-5,6a,7,8,9,10-hexahydro-1′H-spiro[pyrido[1,2-a][1,8]naphthyridine-6,5′-pyrimidine]-2′,4′,6′(3′H)-trione

Starting Material: Intermediate 26.

MS (ES) MH⁺: 407 for C₁₇H₁₉BrN₄O₃.

¹H NMR (300 MHz, DMSO): 0.7 (d, 3H), 0.73 (m, 1H), 0.9 (d, 3H), 1.7 (m, 3H), 2.4 (d, 1H), 2.80 (d, 1H), 3.75 (d, 1H), 4.7 (m, 2H), 4.8 (d, 1H), 7.3 (s, 1H), 8.0 (s, 1H), 11.5 (s, 1H), 11.7 (s, 1H).

### Example 12

### (6aS,7S,9R)-rel-1-chloro-7,9-dimethyl-6a,7,9,10-tetrahydro-1′H,5H-spiro[[1,4]oxazino[4,3-a][1,7]naphthyridine-6,5′-pyrimidine]-2′,4′,6′(3′H)-trione

Starting Material: Intermediate 27.

MS (ES) MH⁺: 365 for C₁₆H₁₇ClN₄O₄.

¹H NMR (300 MHz, DMSO): 0.9 (d, 3H), 1.1 (d, 3H), 2.9 (t, 1H), 3.0 (d, 1H), 3.4 (d, 1H), 3.5 (m, 1H), 3.8 (m, 2H), 4.35 (d, 2H), 6.95 (d, 1H), 7.6 (d, 1H), 11.5 (s, 1H), 11.8 (s, 1H).

### Example 13

### (6aR,7R,9S)-1-chloro-7,9-dimethyl-5,6a,7,8,9,10-hexahydro-1′H-spiro[pyrido[1,2-a][1,7]naphthyridine-6,5′-pyrimidine]-2′,4′,6′(3′H)-trione

Starting Material: Intermediate 28.

MS (ES) MH⁺: 363 for C₁₇H₁₉ClN₄O₃.

¹H NMR (300 MHz, DMSO): 0.65 (d, 3H), 0.9 (d, 3H), 1.8 (m, 2H), 2.0 (m, 1H), 2.64 (t, 1H), 3.0 (d, 1H), 3.42 (d, 1H), 3.65 (d, 1H), 4.14 (d, 1H), 6.9 (d, 1H), 7.6 (d, 1H), 11.4 (s, 1H), 11.75 (s, 1H).

### Example 14

### (6aS,9R)-rel-2-Fluoro-3-iodo-9-methyl-9,10-dihydro-1′H-spiro[pyrazino[1,2-a][1,8]naphthyridine-6,5′-pyrimidine]-2′,4′,6′,7(3′H,5H,6aH,8H)-tetraone

Starting Material: Intermediate 33.

MS (ES) MH⁺: 474 for C₁₅H₁₃FN₅O₄.

¹H NMR (300 MHz, DMSO): 1.1 (d, 3H), 3.1 (m, 1H), 3.35 (d, 1H), 3.5 (m, 1H), 3.9 (m, 2H), 4.3 (d, 1H), 7.80 (d, 1H), 8.1 (s, 1H), 11.5 (s, 1H), 11.7 (s, 1H).

### Example 15

### (6aS,9S)-rel-2-Fluoro-3-iodo-9-methyl-9,10-dihydro-1′H-spiro[pyrazino[1,2-a][1,8]naphthyridine-6,5′-pyrimidine]-2′,4′,6′,7(3′H,5H,6aH,8H)-tetraone

Starting Material: Intermediate 33.

MS (ES) MH⁺: 474 for C₁₅H₁₃FN₅O₄.

¹H NMR (300 MHz, DMSO): 1.1 (m, 3H), 2.8-3.2 (m, 2H), 3.4-3.5 (m, 1H), 3.65 (m, 1H), 4.5 (d, 1H), 4.9 (d, 1H), 7.6 (d, 1H), 8.4 (d, 2H), 11.3 (s, broad, 2H), 11.7 (s, 1H).

### Example 16

### (6aS,7S,9R)-rel-2-Fluoro-7,9-dimethyl-3-(5-methyl-1,3,4-thiadiazol-2-yl)-6a,7,9,10-tetrahydro-1′H,5H-spiro[[1,4]oxazino[4,3-a][1,8]naphthyridine-6,5′-pyrimidine]-2′,4′,6′(3′H)-trione

Starting Material: Intermediate 34.

MS (ES) MH⁺: 447 for C₁₉H₁₉FN₆O₄S.

¹H NMR (300 MHz, DMSO): 0.98 (d, 3H), 1.2 (d, 3H), 2.7 (s, 3H), 2.9 (m, 2H), 3.5 (m, 2H), 3.6 (d, 1H), 3.9 (d, 1H), 4.8 (s, 1H), 8.1 (d, 1H), 11.7 (s, 1H), 11.9 (s, 1H).

### Example 17

### (6aR,7R,9S)-2-Fluoro-7,9-dimethyl-3-(5-methyl-1,3,4-thiadiazol-2-yl)-5,6a,7,8,9,10-hexahydro-1′H-spiro[pyrido[1,2-a][1,8]naphthyridine-6,5′-pyrimidine]-2′,4′,6′(3′H)-trione

Starting Material: Intermediate 35.

MS (ES) MH⁺: 445 for C₂₀H₂₁FN₆O₃S.

¹H NMR (300 MHz, DMSO): 0.7 (d, 3H), 0.9 (d, 3H), 1.5 (m, 1H), 1.6 (m, 2H), 2.5 (d, 1H), 2.7 (s, 3H), 2.9 (d, 1H), 3.6 (d, 1H), 3.8 (d, 1H), 4.7 (d, 1H), 8.0 (d, 1H), 11.6 (s, 1H), 11.8 (s, 1H).

### Example 18

### (6aS,7S,9R)-rel-7,9-Dimethyl-6a,7,9,10-tetrahydro-1′H,5H-spiro[[1,4]oxazino[4,3-a][1,8]naphthyridine-6,5′-pyrimidine]-2′,4′,6′(3′H)-trione

Starting Material: Intermediate 16.

MS (ES) MH⁺: 330 for C₁₆H₁₈N₄O₄.

¹H NMR (300 MHz, DMSO): 0.95 (d, 3H) 1.15 (d, 3H) 2.69 (t, 1H) 2.91 (d, 1H) 3.46 (m, 2H) 3.82 (d, 1H) 5.01 (d, 1H), 6.49 (t, 1H), 7.16 (d, 1H), 7.94 (d, 1H), 11.53 (s, 1H), 11.83 (s, 1 H).

### Example 19

### (2R,4S,4aS)-rel-8-Bromo-2,4-dimethyl-2,4,4a,6-tetrahydro-1H,1′H-spiro[[1,4]oxazino[4,3-a][1,5]naphthyridine-5,5′-pyrimidine]-2′,4′,6′(3′H)-trione

STARTING Material: Intermediate 17.

MS (ES) MH⁺: 410 for C₁₆H₁₇BrN₄O₄.

¹H NMR (300 MHz, DMSO): 0.95 (d, 3H) 1.10 (d, 3H) 2.78 (t, 1H) 2.99 (d, 1H) 3.46 (m, 1H) 3.57 (m, 2H) 3.95 (d, 1H) 7.24 (s, 2H) 11.55 (s, 1H) 11.78 (s, 1H).

### Example 20

### (6aS,7S,9R)-rel-2-Fluoro-7,9-dimethyl-6a,7,9,10-tetrahydro-1′H,5H-spiro[[1,4]oxazino[4,3-a][1,8]naphthyridine-6,5′-pyrimidine]-2′,4′,6′(3′H)-trione

Starting Material: Intermediate 142.

MS (ES) MH⁺: 349 for C₁₆H₁₇FN₄O₄; ¹H NMR (300 MHz, DMSO): 1.0 (d, 3H) 1.1 (d, 3H) 2.7 (t, 1H) 2.9 (d, 1H) 3.4 (m, 3H) 3.8 (d, 1H) 4.7 (d, 1H) 6.1 (d, 1H) 7.3 (t, 1H), 11.6 (s, 1H) 11.9 (s, 1H).

### Example 21

### (6aS,7S,9R)-rel-2-fluoro-7,9-dimethyl-3-(pyridin-2-ylethynyl)-6a,7,9,10-tetrahydro-1′H,5H-spiro[[1,4]oxazino[4,3-a][1,8]naphthyridine-6,5′-pyrimidine]-2′,4′,6′(3′H)-trione

Starting Material: Intermediate 143.

MS (ES) MH⁺: 450 for C₂₃H₂₀FN₅O₄.

¹H NMR (300 MHz, DMSO): 0 (d, 3H) 1.2 (d, 3H) 2.8-2.9 (m, 3H) 3.5 (d, 2H) 3.9 (d, 1H) 4.7 (d, 1H) 7.4 (t, 1H) 7.5 (dd, 2H), 7.8 (d, 1H), 8.6 (d, 1H).

### Example 22

### (6aS,7S,9R)-rel-2-Fluoro-7,9-dimethyl-3-(pyrazin-2-ylethynyl)-6a,7,9,10-tetrahydro-1′H,5H-spiro[[4]oxazino[4,3-a][1,8]naphthyridine-6,5′-pyrimidine]-2′,4′,6′(3′H)-trione

Starting Material: Intermediate 144.

MS (ES) MH⁺: 451 for C₂₂H₁₉FN₆O₄.

¹H NMR (300 MHz, DMSO): 1.0 (d, 3H) 1.2 (d, 3H) 2.8-2.9 (m, 2H) 3.5 (d, 3H) 3.9 (d, 1H) 4.8 (d, 1H) 7.5 (d, 1H) 8.7 (d, 2H), 8.8 (s, 1H), 11.7 (s, 1H), 11.9 (s, 1H).

### Example 23

### (6aS,7S,9R)-rel-2-Fluoro-3-((4-methoxyphenyl)ethynyl)-7,9-dimethyl-6a,7,9,10-tetrahydro-1′H,5H-spiro[[1,4]oxazino[4,3-a][1,8]naphthyridine-6,5′-pyrimidine]-2′,4′,6′(3′H)-trione

Starting Material: Intermediate 145.

MS (ES) MH⁺: 479 for C₂₅H₂₃FN₄O₅.

¹H NMR (300 MHz, DMSO): 1.0 (d, 3H) 1.1 (d, 3H) 2.9 (dd, 2H) 3.5 (m, 3H) 3.8 (s, 3H), 3.9 (d, 1H) 4.7 (d, 1H) 7.0 (d, 2H) 7.4 (m, 3H), 11.7 (s, 1H), 11.9 (s, 1H).

### Example 24

### (6aS,7S,9R)-rel-7,9-Dimethyl-3-(5-methyl-1,3,4-thiadiazol-2-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 115.

MS (ES) MH⁺: 429.1 for C₁₉H₂₀N₆O₄S.

¹H NMR (400 MHz, DMSO) δ: 1.0 (d, 3H), 1.12 (d, 3H), 2.7 (s, 3H), 2.75 (m, 1H), 2.9 (d, 1H), 3.5 (m, 1H), 3.6 (m, 2H), 4.0 (d, 1H), 5.1 (dd, 1H), 7.7 (s, 1H), 8.5 (d, 1H).

### Example 25

### (6aS,7S,9R)-rel-7,9-Dimethyl-3-(1,3-thiazol-2-yl)-6a., 7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 116.

MS (ES) MH⁺: 414.2 for C₁₉H₁₉N₅O₄S.

¹H NMR (400 MHz, DMSO) δ: 1.0 (d, 3H), 1.2 (d, 3H), 2.8 (m, 1H), 2.9 (d, 1H), 3.6 (m, 3H), 3.9 (d, 1H), 5.3 (dd, 1H), 7.6 (d, 1H), 7.7 (s, 1H), 7.8 (d, 1H), 8.5 (d, 1H), 11.6 (s, 1H), 11.9 (s, 1H).

### Example 26

### (6aS,7S,9R)-rel-7,9-Dimethyl-3-(1,3-thiazol-5-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 117.

MS (ES) MH⁺: 414.2 for C₁₉H₁₉N₅O₄S.

¹H NMR (400 MHz, DMSO) δ: 1.0 (d, 3H), 1.15 (d, 3H), 2.7 (m, 1H), 3.0 (d, 1H), 3.5 (m, 3H), 3.90 (d, 1H), 5.02 (dd, 1H), 7.48 (s, 1H), 8.08 (s, 1H), 8.28 (d, 1H), 8.96 (s, 1H), 11.63 (brs, 1H), 11.86 (brs, 1H).

### Example 27

### (6aS,7S,9R)-rel-7,9-Dimethyl-3-(thiophen-2-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 118.

MS (ES) MH⁺: 413.2 (MH) for C₂₀H₂₀N₄O₄S.

¹H NMR (400 MHz, DMSO) δ: 1.0 (d, 3H), 1.2 (d, 3H), 2.5 (m, 1H), 2.9 (d, 1H), 3.5 (m, 3H), 3.9 (d, 1H), 5.0 (dd, 1H), 7.1 (m, 1H), 7.3 (d, 1H), 7.4 (m, 2H), 8.25 (s, 1H), 11.5 (m, 2H).

### Example 28

### (6aS,7S,9R)-rel-3-(1-Benzothiophen-2-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 119.

MS (ES) MH⁺: 463.3 for C₂₄H₂₂N₄O₄S.

¹H NMR (400 MHz, DMSO) δ: 1.0 (d, 3H), 1.2 (d, 3H), 2.7 (m, 1H), 3.0 (d, 1H), 3.5 (m, 3H), 3.9 (d, 1H), 5.1 (d, 1H), 7.4 (m, 2H), 7.6 (d, 1H), 7.8 (d, 1H), 7.9 (d, 1H), 8.4 (d, 1H), 11.6 (s, 1H), 11.9 (s, 1H).

### Example 29

### (6aS,7S,9R)-rel-7,9-Dimethyl-3-(5-methylthiophen-2-yl)-6a,7,9,10-tetrahydro-2′H,5H-spiro[1,4oxazino[4,3-a][1,8]naphthyridine-6,5′-pyrimidine]-2′,4′,6′(1′H,3′H)-trione

Starting Material: Intermediate 120.

MS (ES) MH⁺: 427.0 for C₂₁H₂₂N₄O₄S.

¹H NMR (400 MHz, CDCl₃) δ: 1.1 (d, 3H), 1.25 (d, 3H), 2.5 (s, 3H), 2.8 (m, 1H), 3.1 (d, 1H), 3.25 (d, 1H), 3.6 (m, 1H), 3.7 (m, 1H), 4.0 (d, 1H), 5.1 (dd, 1H), 6.7 (d, 1H), 6.9 (d, 1H), 7.2 (m, 1H), 7.9 (s, broad, 1H), 8.1 (s, broad, 1H), 8.3 (d, 1H).

### Example 30

### (6aS,7S,9R)-rel-7,9-Dimethyl-3-(3-methylthiophen-2-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 121.

MS (ES) MH⁺: 427.2 for C₂₁H₂₂N₄O₄S.

¹H NMR (400 MHz, CDCl₃) δ: 1.15 (d, 3H), 1.3 (d, 3H), 2.3 (s, 3H), 2.8 (m, 1H), 3.1 (d, 1H), 3.3 (d, 1H), 3.65 (m, 1H), 3.8 (m, 1H), 4.1 (d, 1H), 5.2 (dd, 1H), 6.9 (d, 1H), 7.2 (d, 1H), 7.7 (s, 1H), 7.8 (s, 1H), 8.0 (s, 1H), 8.2 (d, 1H).

### Example 31

### (6aS,7S,9R)-rel-7,9-Dimethyl-3-[5-(1H-tetrazol-5-yl)thiophen-2-yl]-6a,7,9,10-tetrahydro-2′H,5H-spiro[1,4-oxazino[4,3-a][1,8]naphthyridine-6,5′-pyrimidine]-2′,4′,6′(1′H,3′H)-tri one

Starting Material: Intermediate 122.

MS (ES) MH⁺: 481.2 for C₂₁H₂₀N₈O₄S.

¹H NMR (400 MHz, DMSO) δ: 1.0 (d, 3H), 1.2 (d, 3H), 2.7 (m, 1H), 3.0 (d, 1H), 3.5 (m, 3H), 3.9 (d, 1H), 5.0 (d, 1H), 7.1 (s, broad 1H), 7.2 (d, 1H), 7.3 (d, 1H), 7.5 (s, 1H), 8.3 (d, 1H), 11.7 (s, broad, 1H), 11.9 (s, broad, 1H).

### Example 32

### (6aS,7S,9R)-rel-7,9-Dimethyl-3-(1-methyl-1H-imidazol-2-yl)-6a., 7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 123.

MS (ES) MH⁺: 411.2 for C₂₀H₂₂N₆O₄.

¹H NMR (400 MHz, DMSO) δ: 1.0 (d, 3H), 1.2 (d, 3H), 2.7 (m, 1H), 2.9 (d, 1H), 3.45-3.5 (m, 3H), 3.7 (s, 3H), 3.9 (d, 1H), 5.0 (dd, 1H), 6.9 (d, 1H), 7.2 (d, 1H), 7.5 (s, 1H), 8.2 (d, 1H), 11.7 (s, broad, 2H).

### Example 33

### (6aS,7S,9R)-rel-7,9-Dimethyl-3-(1-methyl-1H-imidazol-4-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 124.

MS (ES) MH⁺: 411.2 for C₂₀H₂₂N₆O₄.

¹H NMR (400 MHz, DMSO) δ: 1.0 (d, 3H), 1.2 (d, 3H), 2.6 (m, 1H), 2.9 (d, 1H), 3.5 (m, 3H), 3.6 (s, 3H), 3.8 (d, 1H), 5.0 (dd, 1H), 7.4 (s, 1H), 7.5 (s, 1H), 7.6 (s, 1H), 8.3 (d, 1H), 11.7 (s, broad, 2H).

### Example 34

### (6aS,7S,9R)-rel-7,9-Dimethyl-3-(4-methylthiophen-3-yl)-6a,7,9,10-tetrahydro-2′H,5H-spiro[1,4-oxazino[4,3-a][1,8]naphthyridine-6,5′-pyrimidine]-2′,4′,6′(1′H,3′H-trione

Starting Material: Intermediate 125.

MS (ES) MH⁺: 427.2 (MH) for C₂₁H₂₂N₄O₄S.

¹H NMR (400 MHz, CDCl₃) δ: 1.15 (d, 3H), 1.3 (d, 3H), 2.2 (s, 3H), 2.8 (m, 1H), 3.1 (d, 1H), 3.3 (d, 1H), 3.7 (m, 1H), 3.8 (m, 1H), 4.1 (d, 1H), 5.2 (dd, 1H), 7.0 (m, 1H), 7.15 (m, 2H), 7.8 (s, 1H), 8.0 (s, 1H), 8.2 (d, 1H).

### Example 35

### (6aS,7S,9R)-rel-3-(1H-Imidazol-2-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 126.

MS (ES) MH⁺: 397.2 for C₁₉H₂₀N₆O₄.

¹H NMR (400 MHz, DMSO) δ: 1.0 (d, 3H), 1.2 (d, 3H), 2.7 (m, 1H), 2.9 (d, 1H), 3.5 (m, 3H), 3.9 (d, 1H), 5.0 (dd, 1H), 6.9 (s, 1H), 7.1 (s, 1H), 7.65 (s, 1H), 8.5 (d, 1H), 11.85 (s, broad, 2H), 12.25 (s, 1H).

### Example 36

### (6aS,7S,9R)-rel-7,9-Dimethyl-3-[5-(1H-pyrazol-5-yl)thiophen-2-yl]-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 127.

MS (ES) MH⁺: 479.2 for C₂₃H₂₂N₆O₄S.

¹H NMR (400 MHz, DMSO) δ: 1.0 (d, 3H), 1.2 (d, 3H), 2.7 (m, 1H), 3.0 (d, 1H), 3.5 (m, 3H), 3.9 (d, 1H), 5.0 (dd, 1H), 6.6 (s, 1H), 7.2 (d, 1H), 7.3 (d, 1H), 7.5 (d, 1H), 8.3 (d, 1H), 12.0 (m, 2H), 12.85 (s, broad, 1H).

### Example 37

### (6aS,7S,9R)-rel-3-(1H-Imidazol-4-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 128.

MS (ES) MH⁺: 397.0 for C₁₉H₂₀N₆O₄.

¹H NMR (400 MHz, DMSO) δ: 1.0 (d, 3H), 1.15 (d, 3H), 2.7 (m, 1H), 3.0 (d, 1H), 3.5 (m, 3H), 3.9 (d, 1H), 5.0 (d, 1H), 7.5 (d, 2H), 8.05 (s, 1H), 8.35 (s, 1H), 11.55 (s, 1H), 11.8 (s, 1H).

### Example 38

### (6aS,7S,9R)-rel-7,9-Dimethyl-3-(pyridin-2-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 37.

MS (ES) MH⁺: 408.2 for C₂₁H₂₁N₅O₄.

¹H NMR (400 MHz, DMSO) δ: 1.0 (d, 3H), 1.2 (d, 3H), 2.7 (m, 1H), 2.95 (d, 1H), 3.45-3.5 (m, 3H), 3.9 (d, 1H), 5.1 (dd, 1H), 7.2 (m, 1H), 7.8 (m, 2H), 7.9 (d, 1H), 8.5 (q, 1H), 8.7 (d, 1H), 11.8 (s, broad, 2H).

### Example 39

### (6aS,7S,9R)-rel-7,9-Dimethyl-3-(quinolin-2-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 130.

MS (ES) MH⁺: 458.2 for C₂₅H₂₃N₅O₄.

¹H NMR (400 MHz, DMSO) δ: 1.0 (d, 3H), 1.2 (d, 3H), 2.5 (t, 1H), 3.0 (d, 1H), 3.5 (m, 3H), 4.0 (d, 1H), 5.1 (dd, 1H), 7.5 (m, 1H), 7.7 (m, 2H), 7.9 (m, 2H), 8.0 (d, 1H), 8.1 (s, 1H), 8.35 (d, 1H), 8.8 (d, 1H), 11.5 (s, 1H), 11.8 (s, 1H).

### Example 40

### (6aS,7S,9R)-rel-3-(1H-Benzimidazol-2-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 131.

MS (ES) MH⁺: 447.2 for C₂₃H₂₂N₆O₄.

¹H NMR (400 MHz, DMSO) δ: 0.99 (d, 3H), 1.2 (d, 3H), 2.7 (q, 1H), 3.0 (d, 1H), 3.5-3.6 (m, 3H), 3.9 (d, 1H), 5.1 (dd, 1H), 7.5 (m, 1H), 7.7 (m, 1H), 7.9 (m, 2H), 8.0 (d, 1H), 8.1 (s, 1H), 8.3 (d, 1H), 8.4 (d, 1H), 11.6 (s, 1H), 11.9 (s, 1H).

### Example 41

### (6aS,7S,9R)-rel-7,9-Dimethyl-3-(1-methyl-1H-pyrazol-5-yl)-6a., 7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 73.

MS (ES) MH⁺: 411.1 for C₂₀H₂₂N₆O₄.

¹H NMR (400 MHz, DMSO) δ: 1.0 (d, 3H), 1.2 (d, 3H), 2.5 (m, 1H), 2.95 (d, 1H), 3.6-3.7 (m, 3H), 3.8 (s, 3H), 3.9 (d, 1H), 5.0 (dd, 1H), 6.3 (d, 1H), 7.3 (d, 1H), 7.4 (d, 1H), 8.1 (d, 1H), 11.6 (s, 1H), 11.9 (s, 1H).

### Example 42

### (6aS,7S,9R)-rel-3-(5-chloropyridin-2-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 74.

MS (ES) MH⁺: 442.1 for C₂₁H₂₀ClN₅O₄.

¹H NMR (400 MHz, DMSO) δ: 1.0 (d, 3H), 1.15 (d, 3H), 2.7 (m, 1H), 2.9 (d, 1H), 3.5 (m, 3H), 3.9 (d, 1H), 5.0 (d, 1H), 7.85 (m, 3H), 8.6-8.7 (m, 2H).

### Example 43

### (6aS,7S,9R)-rel-3-(4-Methoxypyridin-3-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 75.

MS (ES) MH⁺: 438.2 for C₂₂H₂₃N₅O₅.

¹H NMR (400 MHz, DMSO) δ: 1.0 (d, 3H), 1.15 (d, 3H), 2.7 (m, 1H), 3.0 (m, 1H), 3.4-3.5 (m, 3H), 3.8 (s, 3H), 3.9 (d, 1H), 5.0 (dd, 1H), 7.1 (d, 1H), 7.4 (d, 1H), 8.1 (d, 1H), 8.3 (s, 1H), 8.4 (d, 1H), 11.6 (s, 1H), 11.8 (s, 1H).

### Example 44

### (6aS,7S,9R)-rel-7,9-Dimethyl-3-(1H-pyrazol-4-yl)-6a., 7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 76.

MS (ES) MH⁺: 397.2 for C₁₉H₂₀N₆O₄.

¹H NMR (400 MHz, MeOD) δ: 1.0 (d, 3H), 1.2 (d, 3H), 2.7 (m, 1H), 3.0 (d, 1H), 3.3 (s, 1H), 3.6-3.7 (m, 3H), 3.9 (d, 1H), 7.45 (m, 1H), 7.85 (br, 2H), 7.90 (s, 1H), 8.2 (d, 1H).

### Example 45

### (6aS,7S,9R)-rel-7,9-Dimethyl-3-(pyrimidin-5-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 77.

MS (ES) MH⁺: 409.1 for C₂₀H₂₀N₆O₄.

¹H NMR (400 MHz, DMSO) δ: 1.0 (d, 3H), 1.2 (d, 3H), 2.7 (t, 1H), 3.0 (d, 1H), 3.40-3.55 (m, 3H), 3.9 (d, 1H), 5.0 (dd, 1H), 7.7 (d, 1H), 8.4 (d, 2H), 7.90 (d, 1H), 9.0 (s, 1H), 9.1 (s, 1H), 11.6 (s, 1H), 11.9 (s, 1H).

### Example 46

### (6aS,7S,9R)-rel-7,9-dimethyl-3-(pyridin-3-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 78.

MS (ES) MH⁺: 409.1 for C₂₁H₂₁N₅O₄.

¹H NMR (400 MHz, DMSO) δ: 1.0 (d, 3H), 1.2 (d, 3H), 2.7 (t, 1H), 3.0 (d, 1H), 3.5-3.6 (m, 3H), 3.9 (d, 1H), 5.0 (dd, 1H), 7.45 (dd, 1H), 7.6 (s, 1H), 8.0 (d, 1H), 8.4 (d, 1H), 8.5 (t, 1H), 8.9 (d, 1H), 11.6 (s, 1H), 11.9 (s, 1H).

### Example 47

### (6aS,7S,9R)-rel-7,9-Dimethyl-3-(quinolin-8-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 79.

MS (ES) MH⁺: 458.4 for C₂₅H₂₃N₅O₄.

¹H NMR (400 MHz, DMSO) δ: 1.0 (d, 3H), 1.2 (d, 3H), 2.7 (t, 1H), 3.0 (d, 1H), 3.5-3.6 (m, 3H), 3.9 (d, 1H), 5.1 (dd, 1H), 7.5-7.6 (m, 2H), 7.7 (q, 1H), 7.8 (m, 1H), 7.9 (t, 1H), 8.3 (m, 1H), 8.5 (t, 1H), 8.9 (d, 1H), 11.6 (s, 1H), 11.9 (s, 1H).

### Example 48

### (6aS,7S,9R)-rel-3-(Furan-3-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 80.

MS (ES) MH⁺: 397.2 for C₂₀H₂₀N₄O₅.

¹H NMR (400 MHz, MeOD) δ: 1.0 (d, 3H), 1.2 (d, 3H), 2.7 (t, 1H), 3.0 (d, 1H), 3.5-3.6 (m, 3H), 3.9 (d, 1H), 4.8 (s, 1H), 6.8 (s, 1H), 7.3 (s, 1H), 7.4 (s, 1H), 7.7 (s, 1H), 7.8 (m, 1H), 7.9 (t, 1H), 8.0 (s, 1H).

### Example 49

### (6aS,7S,9R)-rel-3-(3,5-Dimethylisoxazol-4-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 81.

MS (ES) MH⁺: 426.2 for C₂₁H₂₃N₅O₅.

¹H NMR (400 MHz, MeOD) δ: 1.0 (d, 3H), 1.2 (d, 3H), 2.1 (s, 3H), 2.2 (s, 3H), 2.7 (t, 1H), 3.0 (d, 1H), 3.5-3.6 (m, 3H), 3.9 (d, 1H), 4.8 (s, 1H), 7.1 (s, 1H), 7.8 (s, 1H).

### Example 50

### (6aS,7S,9R)-rel-7,9-Dimethyl-3-(pyridin-4-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 82.

MS (ES) MH⁺: 408.2 for C₂₁H₂₁N₅O₄.

¹H NMR (400 MHz, MeOD) δ: 1.0 (d, 3H), 1.2 (d, 3H), 2.7 (t, 1H), 3.0 (d, 1H), 3.5-3.6 (m, 3H), 3.9 (d, 1H), 5.0 (s, 1H), 7.3 (d, 3H), 8.45 (s, 1H), 8.5 (s, 2H).

### Example 51

### (6aS,7S,9R)-rel-7,9-Dimethyl-3-(pyrimidin-2-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 38.

MS (ES) MH⁺: 409.2 for C₂₀H₂₀N₆O₄.

¹H NMR (400 MHz, DMSO) δ: 1.0 (d, 3H), 1.2 (d, 3H), 2.75 (m, 1H), 2.95 (d, 1H), 3.55 (m, 3H), 3.9 (d, 1H), 5.1 (m, 1H), 7.3 (m, 1H), 8.1 (s, 1H), 8.8 (d, 2H), 8.9 (d, 1H).

### Example 52

### (6aS,7S,9R)-rel-7,9-Dimethyl-3-(pyrazin-2-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 39.

MS (ES) MH⁺: 409.2 for C₂₀H₂₀N₆O₄.

¹H NMR (400 MHz, DMSO) δ: 1.0 (d, 3H), 1.2 (d, 3H), 2.8 (m, 1H), 3.0 (d, 1H), 3.6 (m, 3H), 3.9 (d, 1H), 5.1 (dd, 1H), 7.9 (d, 1H), 8.5 (d, 1H), 8.6 (m, 1H), 8.8 (d, 1H), 9.1 (d, 1H), 11.6 (s, broad, 2H).

### Example 53

### (6aS,7S,9R)-rel-3-(1-Benzofuran-2-yl)-7,9-dimethyl-6a,7,9,10-tetrahydro-2′H,5H-spiro[1,4-oxazino[4,3-a][1,7]napthyridine-6,5′-pyrimidine]2′,4′,6′(1′H,3′H)-trione

Starting Material: Intermediate 84.

MS (ES) MH⁺: 447.2 for C₂₄H₂₂N₄O₅.

¹H NMR (400 MHz, DMSO) δ: 1.0 (d, 3H), 1.2 (d, 3H), 3.0 (m, 2H), 3.5 (m, 2H), 3.7 (m, 2H), 4.3 (d, 1H), 7.3 (m, 3H), 7.5 (s, 1H), 7.6 (d, 1H), 7.6 (m, 1H), 8.3 (s, 1H), 11.6 (s, 1H), 11.8 (s, 1H).

### Example 54

### (6aS,7S,9R)-rel-3-(Furan-2-yl)-7,9-dimethyl-6a,7,9,10-tetrahydro-2′H,5H-spiro[1,4-oxazino[4,3-a][1,7]naphthyridine-6,5′-pyrimidine]-2′,4′,6′(1′H3″H)-trione

Starting Material: Intermediate 85.

MS (ES) MH⁺: 397.2 for C₂₀H₂₀N₄O₅.

¹H NMR (400 MHz, DMSO) δ: 1.0 (d, 3H), 1.0 (d, 3H), 2.9 (m, 1H), 3.0 (d, 1H), 3.4 (d, 1H), 3.5 (m, 1H), 3.7 (m, 2H), 4.2 (dd, 1H), 6.55 (m, 1H), 6.8 (m, 1H), 7.2 (s, 1H), 7.7 (m, 1H), 8.2 (s, 1H).

### Example 55

### (6aS,7S,9R)-rel-7,9-Dimethyl-3-(pyridin-2-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 40.

MS (ES) MH⁺: 408.2 for C₂₁H₂₁N₅O₄.

¹H NMR (400 MHz, MeOD) δ: 1.05 (d, 3H), 1.1 (d, 3H), 3.1 (m, 1H), 3.3 (m, 2H), 3.7 (m, 1H), 3.8 (m, 1H), 3.9 (d, 1H), 4.15 (d, 1H), 7.4 (m, 1H), 7.9 (s, 1H), 7.95 (m, 1H), 8.1 (d, 1H), 8.2 (s, 1H), 8.6 (d, 1H).

### Example 56

### (6aS,7S,9R)-rel-7,9-Dimethyl-3-(quinolin-2-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 111.

MS (ES) MH⁺: 458.1 for C₂₅H₂₃N₅O

¹H NMR (400 MHz, DMSO) δ: 1.0 (d, 3H), 1.2 (d, 3H), 3.0 (m, 2H), 3.5 (m, 1H), 3.6 (m, 2H), 3.8 (d, 1H), 4.3 (d, 1H), 7.5 (t, 1H), 7.8 (m, 1H), 8.0 (m, 2H), 8.2 (s, 1H), 8.4 (d, 1H), 8.5 (d, 1H), 11.6 (s, 1H), 11.8 (s, 1H).

### Example 57

### (6aS,7S,9R)-rel-7,9-dimethyl-3-(1-methyl-1H-pyrazol-5-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 86.

MS (ES) MH⁺: 411.2 for C₂₀H₂₂N₆O₄.

¹H NMR (400 MHz, DMSO) δ: 1.0 (d, 3H), 1.2 (d, 3H), 2.9 (m, 1H), 3.0 (d, 1H), 3.4 (d, 1H), 3.5 (m, 1H), 3.7 (m, 2H), 4.0 (s, 3H), 4.3 (d, 1H), 6.5 (d, 1H), 7.3 (s, 1H), 7.4 (d, 1H), 8.3 (s, 1H), 11.55 (s, 1H), 11.8 (s, 1H).

### Example 58

### (6aS,7S,9R)-rel-3-(4-Methoxypyridin-3-yl)-7,9-dimethyl-6a,7,9,10-tetrahydro-2′H,5H-spiro[1,4-oxazino[4,3-a][1,7]naphthyridine-6,5′-pyrimidine]-2′,4′,6′(1″H3″H)-trione

Starting Material: Intermediate 87.

MS (ES) MH⁺: 438.2 for C₂₂H₂₃N₅O₅.

¹H NMR (400 MHz, DMSO) δ: 1.0 (d, 3H), 1.15 (d, 3H), 2.9 (m, 1H), 3.0 (d, 1H), 3.4 (d, 1H), 3.5 (m, 1H), 3.8 (m, 2H), 3.9 (s, 3H), 4.25 (d, 1H), 7.1 (d, 1H), 7.5 (s, 1H), 8.3 (s 1H), 8.4 (d, 1H), 8.8 (s, 1H), 11.5 (s, 1H), 11.8 (s, 1H).

### Example 59

### (6aS,7S,9R)-rel-7,9-Dimethyl-3-(1H-pyrazol-4-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 88.

MS (ES) MH⁺: 397.2 for C₁₉H₂₀N₆O₄.

¹H NMR (400 MHz, DMSO) δ: 0.95 (d, 3H), 1.1 (d, 3H), 2.8 (m, 1H), 3.0 (d, 1H), 3.3 (d, 1H), 3.6 (m, 3H), 4.1 (d, 1H), 7.2 (s, 1H), 7.8 (s, broad, 1H), 8.0 (s, broad, 1H), 8.1 (s, 1H), 11.6 (s, broad, 1H), 12.8 (s, 1H).

### Example 60

### (6aS,7S,9R)-rel-7,9-Dimethyl-3-(pyrimidin-5-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 88.

MS (ES) MH⁺: 409.2 for C₂₀H₂₀N₆O₄.

¹H NMR (400 MHz, DMSO) δ: 1.0 (d, 3H), 1.15 (d, 3H), 2.9 (m, 1H), 3.0 (d, 1H), 3.4 (d, 1H), 3.5 (m, 1H), 3.7 (m, 2H), 4.3 (d, 1H), 7.7 (s, 1H), 8.4 (s, 1H), 9.1 (d, 1H), 9.3 (m, 2H), 11.6 (s, 1H), 11.8 (s, 1H).

### Example 61

### (6aS,7S,9R)-rel-7,9-Dimethyl-3-pyridin-3-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 90.

MS (ES) MH⁺: 408.2 for C₂₁H₂₁N₅O₄.

¹H NMR (400 MHz, DMSO) δ: 1.0 (d, 3H), 1.15 (d, 3H), 2.9 (m, 1H), 3.0 (d, 1H), 3.4 (m, 1H), 3.55 (m, 1H), 3.7 (m, 2H), 4.3 (d, 1H), 7.4 (m, 1H), 7.6 (s, 1H), 8.25 (m, 1H), 8.35 (s, 1H), 8.5 (m, 1H), 9.1 (d, 1H), 11.60 (s, broad, 1H).

### Example 62

### (6aS,7S,9R)-rel-7,9-Dimethyl-3-(quinolin-8-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 91.

MS (ES) MH⁺: 458.2 for C₂₅H₂₃N₅O₄.

¹H NMR (400 MHz, DMSO) δ: 1.0 (d, 3H), 1.2 (d, 3H), 2.9 (m, 1H), 3.05 (d, 1H), 3.4 (d, 1H), 3.5 (m, 1H), 3.7 (m, 2H), 4.3 (d, 1H), 7.6 (m, 1H), 7.7 (m, 1H), 7.95 (m, 2H), 8.1 (m, 1H), 8.4 (m, 1H), 8.9 (m, 1H), 11.45 (s, broad, 2H).

### Example 63

### (6aS,7S,9R)-rel-3-(furan-3-yl)-7,9-dimethyl-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 92.

MS (ES) MH⁺: 397.2 for C₂₀H₂₀N₄O₅.

¹H NMR (400 MHz, DMSO) δ: 0.96 (d, 3H), 1.05 (d, 3H), 2.85 (m, 1H), 3.00 (d, 1H), 3.32 (d, 1H), 3.52 (m, 1H), 3.62 (m, 2H), 4.17 (d, 1H), 6.87 (s, 1H), 7.23 (s, 1H), 7.68 (d, 1H), 8.05 (s, 1H), 8.17 (s, 1H), 11.53 (s, 1H), 11.79 (s, 1H).

### Example 64

### (6aS,7S,9R)-rel-3-(3,5-Dimethylisoxazol-4-yl)-7,9-dimethyl-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 93.

MS (ES) MH⁺: 426.2 for C₂₁H₂₃N₅O₅.

¹H NMR (400 MHz, DMSO) δ: 1.0 (d, 3H), 1.15 (d, 3H), 2.3 (s, 3H), 2.5 (s, 3H), 2.9 (m, 1H), 3.0 (d, 1H), 3.4 (d, 1H), 3.5 (m, 1H), 3.65 (m, 2H), 4.2 (dd, 1H), 7.1 (s, 1H), 8.3 (s, 1H), 12.0 (m, 3H).

### Example 65

### (6aS,7S,9R)-rel-7,9-Dimethyl-3-(pyridin-4-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 94.

MS (ES) MH⁺: 406.2 for C₂₁H₂₁N₅O₄.

¹H NMR (400 MHz, DMSO) δ: 1.0 (d, 3H), 1.2 (d, 3H), 2.9 (m, 1H), 3.0 (d, 1H), 3.4 (d, 1H), 3.5 (m, 1H), 3.7 (m, 1H), 3.7 (d, 1H), 4.3 (d, 1H), 7.7 (s, 1H), 7.9 (d, 2H), 8.4 (s, 1H), 8.6 (m, 2H), 11.6 (s, 1H), 11.85 (s, 1H).

### Example 66

### (6aS,7S,9R)-rel-7,9-dimethyl-3-(1-methyl-1H-pyrazol-4-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 95.

MS (ES) MH⁺: 411.2 for C₂₀H₂₂N₆O₄.

¹H NMR (400 MHz, DMSO) δ: 1.0 (d, 3H), 1.2 (d, 3H), 2.8 (t, 1H), 3.0 (d, 1H), 3.3 (d, 1H), 3.5 (q, 1H), 3.6-3.65 (m, 2H), 3.8 (s, 3H), 3.9 (d, 1H), 4.1 (dd, 1H), 7.2 (s, 1H), 7.8 (s, 1H), 8.0 (s, 1H), 8.1 (s, 1H), 11.5 (s, 1H), 11.8 (s, 1H).

### Example 67

### (6aS,7S,9R)-rel-7,9-dimethyl-3-(pyrazin-2-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 41.

MS (ES) MH⁺: 409.2 for C₂₀H₂₀N₆O₄.

¹H NMR (400 MHz, DMSO) δ: 1.0 (d, 3H), 1.2 (d, 3H), 1.9 (s, 3H), 2.9 (m, 2H), 3.5 (m, 2H), 3.7 (m, 1H), 3.7 (m, 1H), 4.3 (d, 1H), 7.9 (s, 1H), 8.4 (s, 1H), 8.6 (dd, 2H), 9.4 (d, 1H), 11.8 (s, broad, 2H).

### Example 68

### (2R,4S,4aS)-rel-8-(1-Benzofuran-2-yl)-2,4-dimethyl-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 96.

MS (ES) MH⁺: 447.2 for C₂₄H₂₂N₄O₅.

¹H NMR (400 MHz, DMSO) δ: 0.9 (d, 3H), 1.15 (d, 3H), 2.90 (m, 1H), 3.1 (d, 1H), 3.4 (d, 2H), 3.6 (m, 1H), 3.7 (d, 1H), 4.1 (d, 1H), 7.2 (s, 1H), 7.25 (m, 2H), 7.4 (m, 1H), 7.6 (m, 2H), 7.7 (d, 2H), 11.6 (s, 1H), 11.8 (s, 1H).

### Example 69

### (2R,4S,4aS)-rel-2,4-Dimethyl-8-(pyridin-2-yl)-1,2,4,4a-tetrahydro-2′H,6H-spiro[1,4-oxazino[4,3-a][1,5]naphthyridine-5,5′-pyrimidine]-2′,4′,6′(1′H3″H)-trione

Starting Material: Intermediate 42.

MS (ES) MH⁺: 408.2 for C₂₁H₂₁N₅O₄.

¹H NMR (400 MHz, DMSO) δ: 1.1 (d, 3H), 1.2 (d, 3H), 2.85 (m, 1H), 3.2 (m, 1H), 3.5 (m, 2H), 3.6 (m, 1H), 3.7 (d, 1H), 4.1 (d, 1H), 7.3 (d, 1H), 7.35 (d, 1H), 7.8 (t, 1H), 8.1 (m, 2H), 8.55 (s, 1H), 11.6 (s, broad, 2H).

### Example 70

### (2R,4S,4aS)-rel-2,4-Dimethyl-8-(1-methyl-1H-pyrazol-5-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 97.

MS (ES) MH⁺: 411.2 for C₂₀H₂₂N₆O₄.

¹H NMR (400 MHz, DMSO-d₆) δ: 0.95 (d, 3H), 1.1 (d, 3H), 2.8 (t, 1H), 3.1 (d, 1H), 3.4 (d, 1H), 3.45 (m, 1H), 3.6 (m, 1H), 3.7 (d, 1H), 4.0 (s, 3H), 4.05 (m, 1H), 6.5 (d, 1H), 7.3 (d, 1H), 7.4 (d, 1H), 7.4 (d, 1H), 11.5 (s, 1H), 11.8 (s, 1H).

### Example 71

### (2R,4S,4aS)-rel-8-(4-Methoxypyridin-3-yl)-2,4-dimethyl-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 98.

MS (ES) (M-H)⁻: 436.2 for C₂₂H₂₃N₅O₅.

¹H NMR (400 MHz, DMSO-d₆) δ: 1.0 (d, 3H), 1.1 (d, 3H), 2.8 (q, 1H), 3.1 (d, 1H), 3.4 (d, 1H), 3.45 (, 1H), 3.5 (q, 1H), 3.7 (m, 2H), 3.9 (s, 3H), 4.0 (dd, 1H), 7.1 (d, 1H), 7.3 (d, 1H), 7.6 (d, 1H), 8.4 (d, 1H), 8.7 (d, 1H), 11.5 (s, 1H), 11.8 (s, 1H).

### Example 72

### (2R,4S,4aS)-rel-2,4-Dimethyl-8-(1H-pyrazol-4-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 99.

MS (ES) MH⁺: 438.2 for C₂₂H₂₃N₅O₅.

¹H NMR (400 MHz, DMSO-d₆) δ: 1.0 (d, 3H), 1.1 (d, 3H), 2.8 (t, 1H), 3.1 (d, 1H), 3.3 (s, 1H), 3.4 (q, 1H), 3.6 (m, 2H), 3.5 (q, 1H), 3.7 (m, 2H), 4.0 (dd, 1H), 7.2 (d, 1H), 7.4 (d, 1H), 7.85 (br, 1H), 8.0 (br, 1H), 8.3 (d, 1H), 11.5-11.8 (s, broad, 2H), 12.8 (s, 1H).

### Example 73

### (2R,4S,4aS)-rel-2,4-Dimethyl-8-(pyrimidin-5-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 100.

MS (ES) MH⁺: 409.2 for C₂₀H₂₀N₆O₄.

¹H NMR (400 MHz, DMSO-d₆) δ: 1.0 (d, 3H), 1.1 (d, 3H), 2.8 (t, 1H), 3.1 (d, 1H), 3.3 (s, 1H), 3.4 (q, 1H), 3.6 (m, 2H), 3.5 (q, 1H), 3.7 (d, 2H), 4.1 (dd, 1H), 7.4 (d, 1H), 7.9 (d, 1H), 9.0 (s, 1H), 9.2 (s, 2H), 11.6 (s, 1H), 11.8 (s, 1H).

### Example 74

### (2R,4S,4aS)-rel-2,4-Dimethyl-8-(pyridin-3-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 101.

MS (ES) MH⁺: 408.1 for C₂₁H₂₁N₅O₄.

¹H NMR (400 MHz, DMSO) δ: 1.0 (d, 3H), 1.2 (d, 3H), 2.8 (t, 1H), 3.1 (d, 1H), 3.3-3.4 (m, 3H), 3.7 (m, 1H), 4.1 (m, 1H), 7.4 (m, 2H), 7.8 (d, 1H), 8.2 (d, 1H), 8.4 (d, 1H), 9.1 (s, 1H), 11.55 (s, 1H), 11.8 (s, 1H).

### Example 75

### (2R,4S,4aS)-rel-2,4-Dimethyl-8-(quinolin-8-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 102.

MS (ES) MH⁺: 458.0 for C₂₅H₂₃N₅O₄.

¹H NMR (400 MHz, DMSO) δ: 1.0 (d, 3H), 1.2 (d, 3H), 2.9 (m, 1H), 3.05 (d, 1H), 3.4 (d, 1H), 3.5 (m, 1H), 3.7 (m, 2H), 4.1 (d, 1H), 7.35 (t, 1H), 7.55 (q, 1H), 7.65 (t, 2H), 7.9 (d, 1H), 8.1 (m, 2H), 8.4 (m, 1H), 8.9 (m, 1H), 11.55 (s, 1H), 11.8 (s, 1H).

### Example 76

### (2R,4S,4aS)-rel-8-(Furan-3-yl)-2,4-dimethyl-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 104.

MS (ES) MH⁺: 397.1 for C₂₀H₂₀N₄O₅.

¹H NMR (400 MHz, DMSO-d₆) δ 1.0 (d, 3H), 1.1 (d, 3H), 2.8 (t, 1H), 3.1 (d, 1H), 3.5 (d, 1H), 3.6 (d, 1H), 4.0 (d, 1H), 6.9 (s, 1H), 7.2 (d, 1H), 7.4 (d, 1H), 7.65 (s, 1H), 8.05 (s, 1H), 11.5 (s, 1H0, 11.8 (s, 1H).

### Example 77

### (2R,4S,4aS)-rel-8-(3,5-Dimethylisoxazol-4-yl)-2,4-dimethyl-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 103.

MS (ES) MH⁺: 426.2 for C₂₁H₂₃N₅O₅.

¹H NMR (400 MHz, DMSO) δ: 0.9 (d, 3H), 1.15 (d, 3H), 2.90 (m, 1H), 3.1 (d, 1H), 3.4 (d, 2H), 3.6 (m, 1H), 3.7 (d, 1H), 4.1 (d, 1H), 7.2 (s, 1H), 7.25 (m, 2H), 7.4 (m, 1H), 7.6 (m, 2H), 7.7 (d, 2H), 11.6 (s, 1H), 11.8 (s, 1H).

### Example 78

### (2R,4S,4aS)-rel-2,4-dimethyl-8-(pyridin-4-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 106.

MS (ES) MH⁺: 408.2 for C₂₁H₂₁N₅O₄.

¹H NMR (400 MHz, DMSO) δ: 1.0 (d, 3H), 1.15 (d, 3H), 2.9 (m, 1H), 3.15 (d, 1H), 3.5 (m, 2H), 3.6 (m, 1H), 3.7 (d, 1H), 4.1 (d, 1H), 7.3 (d, 1H), 7.9 (dd, 3H), 8.5 (s, 2H), 11.6 (s, 1H), 11.8 (s, 1H).

### Example 79

### (2R,4S,4aS)-rel-2,4-Dimethyl-8-(1-methyl-1H-pyrazol-4-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 105.

MS (ES) MH⁺: 411.2 for C₂₀H₂₂N₆O₄.

¹H NMR (400 MHz, CD₃OD) δ: 1.2 (d, 3H), 1.2 (d, 3H), 2.9 (t, 1H), 3.2 (d, 2H), 3.6 (q, 1H), 3.6 (m, 2H), 3.9 (s, 3H), 4.0 (d, 1H), 7.2 (d, 1H), 7.4 (d, 1H), 7.85 (d, 1H), 7.95 (s, 1H).

### Example 80

### (2R,4S,4aS)-rel-2,4-Dimethyl-8-(pyrazin-2-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 43.

MS (ES) MH⁺: 409.2 for C₂₀H₂₀N₆O₄.

¹H NMR (400 MHz, DMSO) δ: 1.0 (d, 3H), 1.2 (d, 3H), 2.9 (t, 1H), 3.1 (d, 1H), 3.5 (dd, 1H), 3.6 (m, 1H), 3.75 (d, 1H) 4.15 (d, 1H), 7.4 (d, 1H), 8.1 (d, 1H), 8.6 (dd, 2H), 9.3 (s, 1H), 11.6 (s, 1H), 11.8 (s, 1H).

### Example 81

### (6aS,7S,9R)-rel-3-(4-Fluorophenyl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 44.

MS (ES) MH⁺: 425.1 for C₂₂H₂₁FN₄O₄.

¹H NMR (400 MHz, CD₃OD) δ: 1.0 (d, 3H), 1.1 (d, 3H), 2.7 (t, 1H), 3.1 (d, 2H), 3.2 (s, 1H), 3.5 (m, 2H), 3.9 (d, 1H), 4.9 (d, 1H), 6.9 (d, 1H), 7.2 (m, 1H), 7.3-7.4 (m, 2H), 7.45 (s, 1H), 8.25 (s, 1H).

### Example 82

### (6aS,7S,9R)-rel-7,9-Dimethyl-3-(2-methylphenyl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 107.

MS (ES) MH⁺: 421.1 for C₂₃H₂₄N₄O₄.

¹H NMR (400 MHz, CDCl₃) δ: 1.2 (d, 3H), 1.3 (d, 3H), 2.3 (s, 3H), 2.8 (t, 1H), 3.0 (d, 1H), 3.3 (d, 1H), 3.6 (m, 1H), 3.8 (m, 1H), 4.0 (d, 1H), 5.15 (dd, 1H), 7.1 (s, 1H), 7.2 (q, 1H), 7.2-7.3 (m, 3H), 8.1 (d, 2H), 8.4 (s, 1H).

### Example 83

### (6aS,7S,9R)-rel-3-(2-Methoxyphenyl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 108.

MS (ES) MH⁺: 437.1 for C₂₃H₂₄N₄O₅.

¹H NMR (400 MHz, MeOD) δ: 1.0 (d, 3H), 1.1 (d, 3H), 2.7 (t, 3H), 3.0 (d, 1H), 3.1 (s, 1H), 3.5 (m, 1H), 3.9 (d, 1H), 4.8 (s, 1H), 6.9 (m, 2H), 7.1-7.2 (m, 2H), 7.3 (q, 1H), 8.0 (q, 1H).

### Example 84

### (6aS,7S,9R)-rel-7,9-Dimethyl-3-phenyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 109.

MS (ES) MH⁺: 407.2 for C₂₂H₂₂N₄O₄.

¹H NMR (400 MHz, DMSO) δ: 1.0 (d, 3H), 1.15 (d, 3H), 2.7 (t, 1H), 3.0 (d, 1H), 3.4-3.6 (m, 1H), 3.9 (d, 1H), 5.0 (dd, 1H), 7.3 (t, 1H), 7.4 (t, 2H), 7.5 (m, 2H), 8.3 (d, 1H).

### Example 85

### (2R,4S,4aS)-rel-2,4-Dimethyl-8-(1,3-thiazol-2-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 45.

MS (ES) MH⁺: 414.2 for C₁₉H₁₉N₄O₅S.

¹H NMR (400 MHz, CD₃OD) δ: 1.1 (d, 3H), 1.2 (d, 3H), 3.0 (dd, 1H), 3.65 (m, 1H), 3.8 (m, 1H), 3.9 (d, 1H), 4.1 (d, 1H), 7.3 (d, 1H), 7.5 (d, 1H), 7.8 (d, 1H), 7.9 (d, 1H).

### Example 86

### (6aS,7S,9R)-rel-7,9-Dimethyl-3-(1,3-thiazol-2-yl)-6a., 7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 46.

MS (ES) MH⁺: 414.2 for C₁₉H₁₉N₅O₄S.

¹H NMR (400 MHz, DMSO) δ: 1.1 (d, 3H), 1.15 (d, 3H), 2.9 (m, 1H), 3.5 (m, 2H), 3.65 (m, 1H), 3.75 (d, 1H), 4.3 (d, 1H), 7.6 (d, 1H), 7.7 (s, 1H), 7.8 (d, 1H), 8.3 (s, 1H), 11.7 (s, broad, 2H).

### Example 87

### (6aS,7S,9R)-rel-3-(1,3-Benzothiazol-2-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 47.

MS (ES) MH⁺: 464.2 for C₂₃H₂₁N₅O₄S.

¹H NMR (400 MHz, DMSO) δ: 1.0 (d, 3H), 1.2 (d, 3H), 2.8 (m, 1H), 3.0 (d, 1H), 3.5 (m, 3H), 4.0 (d, 1H), 5.1 (d, 1H), 7.4 (t, 1H), 7.5 (t, 1H), 7.85 (s, 1H), 7.9 (d, 1H), 8.1 (d, 1H), 8.6 (d, 1H), 11.6 (s, 1H), 11.9 (s, 2H).

### Example 88

### (2R,4S,4aS)-rel-8-(1,3-Benzothiazol-2-yl)-2,4-dimethyl-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 48.

MS (ES) MH⁺: 464.0 for C₂₃H₂₁N₅O₄S.

¹H NMR (400 MHz, MeOD) δ: 1.1 (d, 3H), 1.2 (d, 3H), 3.0 (dd, 1H), 3.3 (d, 1H), 3.4 (d, 1H), 3.65 (m, 1H), 3.8 (m, 1H), 3.9 (d, 1H), 4.0 (dd, 1H), 7.2 (d, 1H), 7.3 (t, 1H), 7.4 (t, 1H), 7.9 (d, 1H), 7.9 (d, 1H), 8.0 (d, 1H).

### Example 89

### (6aS,7S,9R)-rel-3-(1,3-benzothiazol-2-yl)-7,9-dimethyl-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 49.

MS (ES) MH⁺: 464.1 for C₂₃H₂₁N₅O₄S.

¹H NMR (400 MHz, DMSO) δ: 1.0 (d, 3H), 1.2 (d, 3H), 2.9 (m, 2H), 3.6 (m, 3H), 3.8 (d, 1H), 4.4 (d, 1H), 7.4 (m, 1H), 7.5 (m, 1H), 7.9 (s, 1H), 7.9 (d, 1H), 8.1 (d, 1H), 8.35 (s, 1H), 11.6 (s, 1H), 11.9 (s, 1H).

### Example 90

### (6aS,7S,9R)-rel-7,9-Dimethyl-3-(1-methyl-1H-imidazol-5-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 50.

MS (ES) MH⁺: 411.1 for C₂₀H₂₂N₆O₄. ¹H NMR (400 MHz, MeOD) δ: 1.1 (d, 3H), 1.2 (d, 3H), 2.8 (m, 1H), 3.1 (d, 1H), 3.6 (m, 1H), 3.6 (s, 3H), 3.7 (m, 1H), 4.0 (d, 1H), 5.0 (d, 1H), 6.95 (s, 1H), 7.3 (s, 1H), 7.7 (s, 1H), 8.0 (d, 1H).

### Example 91

### (2R,4S,4aS)-rel-2,4-Dimethyl-8-(1-methyl-1H-imidazol-5-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 51.

MS (ES) MH⁺: 411.1 for C₂₀H₂₂N₆O₄.

¹H NMR (400 MHz, DMSO) δ: 1.0 (d, 3H), 1.2 (d, 3H), 2.8 (m, 1H), 3.1 (d, 1H), 3.4 (m, 1H), 3.6 (m, 1H), 3.7 (d, 1H), 4.0 (s, 3H), 4.1 (d, 1H), 7.4 (d, 1H), 7.5 (d, 1H), 7.8 (s, 1H), 8.8 (s, 1H), 11.6 (s, 1H), 11.8 (s, 1H).

### Example 92

### (6aS,7S,9R)-rel-3-(3-Methoxypyrazin-2-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 52.

MS (ES) MH⁺: 439.1 for C₂₁H₂₂N₆O₅.

¹H NMR (400 MHz, DMSO) δ: 1.0 (d, 3H), 1.2 (d, 3H), 2.7 (m, 1H), 3.0 (d, 1H), 3.5 (m, 3H), 3.9 (d, 1H), 5.1 (dd, 1H), 7.9 (s, 1H), 8.0 (d, 1H), 8.2 (d, 1H), 8.8 (d, 1H), 11.6 (s, 1H), 11.9 (s, 1H).

### Example 93

### (6aS,7S,9R)-rel-3-(3-Methoxypyrazin-2-yl)-7,9-dimethyl-6a,7,9,10-tetrahydro-2′H,5H-spiro[1,4-oxazino[4,3-a][1,7]naphthyridine-6,5′-pyrimidine]-2′,4′,6′(1′H, 3H)-trione

Starting Material: Intermediate 53.

MS (ES) MH⁺: 439.1 for C₂₁H₂₂N₆O₅.

¹H NMR (400 MHz, DMSO) δ: 0.9 (d, 3H), 1.2 (d, 3H), 2.95 (m, 2H), 3.5-3.7 (m, 3H), 3.9 (s, 3H), 4.3 (d, 1H), 7.5 (s, 1H), 8.2 (t, 1H), 8.2 (d, 1H), 8.3 (s, 1H), 10.7 (s, broad, 2H).

### Example 94

### (6aS,7S,9R)-rel-7,9-Dimethyl-3-(pyridazin-4-yl)-6a,7,9,10-tetrahydro-2′H,5H-spiro[1,4-oxazino[4,3-a][1,8]naphthyridine-6,5′-pyrimidine]-2′,4′,6′(1′H, 3H)-trione

Starting Material: Intermediate 54.

MS (ES) MH⁺: 409.1 for C₂₀H₂₀N₆O₄.

¹H NMR (400 MHz, MeOD) δ: 1.1 (d, 3H), 1.3 (d, 3H), 2.8 (t, 1H), 3.1 (d, 1H), 3.2 (m, 1H), 3.7 (m, 1H), 4.1 (d, 1H), 5.1 (d, 1H), 7.4 (s, 1H), 7.9 (d, 1H), 8.5 (s, 1H), 9.1 (d, 1H), 9.5 (s, 1H).

### Example 95

### (2R,4S,4aS)-rel-2,4-Dimethyl-8-(pyridazin-4-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 55.

MS (ES) MH⁺: 409.1 for C₂₀H₂₀N₆O₄.

¹H NMR (400 MHz, MeOD) δ: 1.0 (d, 3H), 1.2 (d, 3H), 2.9 (m, 1H), 3.3 (d, 1H), 3.5 (m, 1H), 3.6 (m, 1H), 3.8 (d, 1H), 4.0 (dd, 1H), 7.3 (d, 1H), 7.9 (t, 1H), 8.2 (dd, 1H), 9.1 (dd, 1H), 9.8 (dd, 1H), 11.6 (s, 1H), 11.9 (s, 1H).

### Example 96

### (6aS,7S,9R)-rel-7,9-Dimethyl-3-(pyridazin-4-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 56.

MS (ES) (M-H)⁻: 407.2 for C₂₀H₂₀N₆O₄.

¹H NMR (400 MHz, DMSO) δ: 1.0 (d, 3H), 1.2 (d, 3H), 2.9 (m, 1H), 3.0 (d, 1H), 3.55 (m, 3H), 3.8 (d, 1H), 4.4 (d, 1H), 7.8 (s, 1H), 8.1 (m, 1H), 8.4 (s, 1H), 9.2 (m, 1H), 9.75 (m, 1H), 11.7 (s, broad, 2H).

### Example 97

### (6aS,7S,9R)-rel-7,9-Dimethyl-3-(1,3-oxazol-2-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 57.

MS (ES) MH⁺: 398.2 for C₁₉H₁₉N₅O₅.

¹H NMR (400 MHz, CDCl₃) δ: 1.0 (d, 3H), 1.2 (d, 3H), 2.75 (dd, 1H), 2.9 (d, 1H), 3.5 (m, 3H), 3.9 (d, 1H), 5.0 (dd, 1H), 7.3 (s, 1H), 7.7 (s, 1H), 8.1 (d, 1H), 8.6 (d, 1H), 11.6 (s, 1H), 11.9 (s, 1H).

### Example 98

### (2R,4S,4aS)-rel-2,4-Dimethyl-8-(1,3-oxazol-2-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 58.

MS (ES) MH⁺: 398.2 for C₁₉H₁₉N₅O₅.

¹H NMR (400 MHz, DMSO) δ: 1.0 (d, 3H), 1.2 (d, 3H), 2.9 (dd, 1H), 3.1 (d, 1H), 3.4 (m, 2H), 3.6 (m, 1H), 3.75 (d, 1H), 4.1 (d, 1H), 7.3 (d, 1H), 7.35 (d, 1H), 7.8 (d, 1H), 8.1 (d, 1H), 11.6 (s, 1H), 11.8 (s, 1H).

### Example 99

### (6aS,7S,9R)-rel-7,9-Dimethyl-3-(1,3-oxazol-2-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 59.

MS (ES) MH⁺: 398.2 for C₁₉H₁₉N₅O₅.

¹H NMR (400 MHz, CDCl₃) δ: 1.1 (d, 3H), 1.2 (d, 3H), 3.05 (m, 1H), 3.2 (m, 2H), 3.6 (m, 1H), 3.8 (m, 1H), 3.9 (d, 1H), 4.1 (d, 1H), 7.2 (s, 1H), 7.6 (s, 1H), 7.8 (s, 1H), 8.1 (s, 1H).

### Example 100

### (6aS,7S,9R)-rel-7,9-Dimethyl-3-(1,3-thiazol-4-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 60.

MS (ES) MH⁺: 414.2 for C₁₉H₁₉N₅O₄S.

¹H NMR (400 MHz, MeOD) δ: 1.1 (d, 3H), 1.2 (d, 3H), 2.8 (dd, 1H), 3.1 (d, 1H), 3.7 (m, 2H), 4.0 (d, 1H), 5.0 (d, 1H), 5.5 (s, 1H), 7.7 (s, 1H), 7.75 (s, 1H), 8.6 (s, 1H), 9.0 (s, 1H).

### Example 101

### (2R,4S,4aS)-rel-2,4-Dimethyl-8-(1,3-thiazol-4-yl)-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 61.

MS (ES) MH⁺: 414.2 for C₁₉H₁₉N₅O₄S.

¹H NMR (400 MHz, MeOD) δ: 1.0 (d, 3H), 1.2 (d, 3H), 2.9 (dd, 1H), 3.3 (d, 1H), 3.6 (m, 1H), 3.7 (m, 1H), 3.8 (d, 1H), 4.1 (d, 1H), 7.3 (d, 1H), 7.9 (m, 2H), 9.0 (s, 1H).

### Example 102

### (6aS,7S,9R)-rel-7,9-Dimethyl-3-(1,3-thiazol-4-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 62.

MS (ES) MH⁺: 414.2 for C₁₉H₁₉N₅O₄S. ¹H NMR (400 MHz, DMSO) δ: 1.0 (d, 3H), 1.2 (d, 3H), 2.9 (m, 1H), 3.0 (d, 1H), 3.5 (m, 2H), 3.7 (m, 2H), 4.25 (d, 1H), 7.65 (s, 1H), 8.0 (m, 1H), 8.3 (s, 1H), 9.14 (m, 1H), 11.55 (s, broad, 2H).

### Example 103

### (6aS,7S,9R)-rel-3-(6-Methoxypyrazin-2-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 63.

MS (ES) MH⁺: 439.2 for C₂₁H₂₂N₆O₅.

¹H NMR (400 MHz, DMSO) δ: 1.0 (d, 3H), 1.2 (d, 3H), 2.7 (m, 1H), 3.0 (d, 1H), 3.5 (m, 3H), 3.9 (d, 1H), 4.0 (s, 3H), 5.1 (d, 1H), 8.0 (s, 1H), 8.1 (s, 1H), 8.7 (s, 1H), 8.8 (d, 1H), 11.7 (s, broad, 2H).

### Example 104

### (2R,4S,4aS)-rel-8-(6-Methoxypyrazin-2-yl)-2,4-dimethyl-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 64.

MS (ES) MH⁺: 439.1 for C₂₁H₂₂N₆O₅.

¹H NMR (400 MHz, DMSO) δ: 1.0 (d, 3H), 1.2 (d, 3H), 2.9 (m, 1H), 3.2 (m, 1H), 3.5 (m, 2H), 3.6 (m, 1H), 3.75 (d, 1H), 4.0 (s, 3H), 4.1 (d, 1H), 7.4 (d, 1H), 8.0 (d, 1H), 8.15 (s, 1H), 8.9 (s, 1H), 11.6 (s, 1H), 11.8 (s, 1H).

### Example 105

### (6aS,7S,9R)-rel-3-(6-Methoxypyrazin-2-yl)-7,9-dimethyl-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 65.

MS (ES) MH⁺: 414.2 for C₁₉H₁₉N₅O₄S.

¹H NMR (400 MHz, DMSO) δ: 1.0 (d, 3H), 1.2 (d, 3H), 2.9 (m, 2H), 3.5 (m, 2H), 3.55 (m, 1H), 3.7 (d, 1H), 4.0 (s, 3H), 4.3 (d, 1H), 7.9 (s, 1H), 8.2 (s, 1H), 8.3 (s, 1H), 9.0 (s, 1H), 11.6 (s, broad, 2H).

### Example 106

### (6aS,7S,9R)-rel-3-[2-(Dimethylamino)pyrimidin-5-yl]-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 66.

MS (ES) MH⁺: 452.2 for C₂₂H₂₅N₇O₄.

¹H NMR (400 MHz, DMSO) δ: 0.99 (d, 3H), 1.2 (d, 3H), 2.7 (m, 1H), 3.0 (d, 1H), 3.1 (s, 6H), 3.4 (m, 1H), 3.45-3.55 (m, 2H), 3.9 (d, 1H), 5.0 (dd, 1H), 7.5 (d, 1H), 8.2 (d, 1H), 8.6 (s, 2H), 11.6 (s, 1H), 11.85 (s, 1H).

### Example 107

### (6aS,7S,9R)-rel-3-(2-Methoxy-1,3-thiazol-4-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 67.

MS (ES) MH⁺: 444.2 for C₂₀H₂₁N₅O₅S.

¹H NMR (400 MHz, MeOD) δ: 1.1 (d, 3H), 1.2 (d, 3H), 2.8 (dd, 1H), 3.1 (d, 1H), 3.3 (m, 1H), 3.6 (m, 2H), 4.0 (d, 1H), 4.1 (s, 3H), 5.0 (d, 1H), 7.0 (s, 1H), 7.7 (s, 1H), 8.5 (d, 1H).

### Example 108

### (6aS,7S,9R)-rel-3-(2-Methoxy-1,3-thiazol-4-yl)-7,9-dimethyl-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 112.

MS (ES) MH⁺: 447.2 for C₂₀H₂₁N₅O₅S.

¹H NMR (400 MHz, DMSO) δ: 1.0 (d, 3H), 1.2 (d, 3H), 2.9 (m, 2H), 3.5 (m, 2H), 3.7 (m, 2H), 4.1 (s, 3H), 4.2 (d, 1H), 7.3 (s, 1H), 7.45 (s, 1H), 8.2 (s, 1H), 11.5 (s, 1H), 11.8 (s, 1H).

### Example 108

### (6aS,7S,9R)-rel-3-(2,4-Dimethyl-1,3-thiazol-5-yl)-7,9-dimethyl-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 133.

MS (ES) MH⁺: 442.2 for C₂₁H₂₃N₅O₄S.

¹H NMR (400 MHz, DMSO) δ: 1.0 (d, 3H), 1.2 (d, 3H), 2.0 (s, 3H), 2.4 (s, 3H), 2.7 (m, 1H), 2.9 (d, 1H), 3.4-3.55 (m, 3H), 3.9 (d, 1H), 5.0 (dd, 1H), 7.25 (d, 1H), 8.0 (d, 1H), 11.6 (s, 1H), 11.85 (s, 1H).

### Example 109

### (2R,4S,4aS)-rel-8-(2,4-Dimethyl-1,3-thiazol-5-yl)-2,4-dimethyl-1,2,4,4a-tetrahydro-2′H,6H-spiro[1,4-oxazino[4,3-a][1,5]naphthyridine-5,5′-pyrimidine]-2′,4′,6′(1′H,3′H)-trione

Starting Material: Intermediate 69.

MS (ES) MH⁺: 442.2 for C₂₁H₂₃N₅O₄S; ¹H NMR (400 MHz, DMSO) δ: 1.0 (d, 3H), 1.2 (d, 3H), 2.5 (s, 3H), 2.6 (s, 3H), 3.0 (d, 1H), 3.5 (m, 2H), 3.6 (m, 1H), 4.2 (d, 1H), 7.2 (s, 1H), 8.2 (s, 1H), 11.55 (s, 1H), 11.8 (s, 1H).

### Example 111

### (6aS,7S,9R)-rel-7,9-Dimethyl-3-[6-(morpholin-4-yl)pyridin-3-yl]-6a,7,9,10-tetrahydro-2′H,5H-spiro[1,4-oxazino[4,3-a][1,8]naphthyridine-6,5′-pyrimidine]-2′,4′,6′(1′H,3′H)-trione

Starting Material: Intermediate 70.

MS (ES) MH⁺: 493.2 for C₂₅H₂₈N₆O₅.

¹H NMR (400 MHz, DMSO) δ: 1.0 (d, 3H), 1.2 (d, 3H), 2.7 (m, 1H), 3.0 (d, 1H), 3.4-3.6 (m, 7H), 3.6 (m, 4H), 3.9 (d, 1H), 5.0 (d, 1H), 6.9 (d, 1H), 7.5 (s, 1H), 7.9 (dd, 1H), 8.2 (d, 1H), 8.3 (d, 1H), 11.55 (s, 1H), 11.8 (s, 1H).

### Example 112

### (6aS,7S,9R)-rel-7,9-Dimethyl-3-[6-(morpholin-4-yl)pyridin-3-yl]-6a,7,9,10-tetrahydro-2′H,5H-spiro[1,4-oxazino[4,3-a][1,7]naphthyridine-6,5′-pyrimidine]-2′,4′,6′(1′H,3′H)-trione

Starting Material: Intermediate 71.

MS (ES) MH⁺: 493.2 for C₂₅H₂₈N₆O₅.

¹H NMR (400 MHz, DMSO) δ: 1.0 (d, 3H), 1.2 (d, 3H), 2.9 (m, 1H), 3.0 (d, 1H), 3.5-3.7 (m, 12H), 4.2 (d, 1H), 6.9 (d, 1H), 7.4 (s, 1H), 8.0 (d, 1H), 8.3 (s, 1H), 8.7 (s, 1H,) 11.55 (s, 1H), 11.8 (s, 1H).

### Example 113

### (6aS,7S,9R)-rel-7,9-Dimethyl-2′,4′,6′-trioxo-1′,3′,4′,6′,6a,7,9,10-octahydro-2′H,5H-spiro[1,4-oxazino[4,3-a][1,8]naphthyridine-6,5′-pyrimidine]-3-carbonitrile

Starting Material: Intermediate 113.

MS (ES) MH⁺: 356.4 for C₁₇H₁₇N₅O₄.

¹H NMR (400 MHz, D₂O) δ: 1.1 (d, 3H), 1.2 (d, 3H), 2.8 (q, 1H), 3.0 (d, 1H), 3.6 (m, 2H), 4.1 (d, 1H), 5.2 (d, 1H), 7.4 (s, 1H), 8.3 (d, 1H).

### Example 114

### (2R,4S,4aS)-rel-8-Bromo-10-chloro-9-fluoro-2,4-dimethyl-2,4,4a,6-tetrahydro-1H,1′H-spiro[[1,4]oxazino[4,3-a][1,5]naphthyridine-5,5′-pyrimidine]-2′,4′,6′(3′H)-trione

A solution of 6-bromo-4-chloro-3-((2R,6S)-2,6-dimethylmorpholino)-5-fluoropicolinaldehyde (Intermediate 9, 110 mg, 0.31 mmol) and barbituric acid (40.1 mg, 0.31 mmol) in AcOH (1.5 ml) and water (1 ml) was heated at 120° C. for 1 hour in a microwave reactor. Solvent was removed and the residue was taken up in EtOAc and again solvent was removed to give a solid. The solid was chromatographed on silica gel (100% CH₂Cl₂ with gradient elution to 50% EtOAc in CH₂Cl₂) to give 35 mg of product as a white solid.

MS (ES) MH⁺: 463 for C₁₆H₁₆BrClFN₄O₄.

¹H NMR (300 MHz, DMSO-d6) δ: 0.9 (d, 3H), 1.1 (d, 3H), 2.9-3.0 (dd, 1H), 3.15 (d, 1H), 3.5-3.7 (m, 2H), 3.8-3.9 (m, 2H), 4.3 (d, 1H), 11.6 (s, 1H), 11.9 (s, 2H).

### Example 115

### (2R,4S,4aS)-rel-9-chloro-2,4-dimethyl-1,2,4,4a-tetrahydro-2′H,6H-spiro[1,4-oxazino[4,3-a][1,6]naphthyridine-5,5′-pyrimidine]-2′,4′,6′(1′H,3′H)-trione

The title compound was synthesized from pyrimidine-2,4,6(1H,3H,5H)-trione and Intermediate 30 using a procedure similar to the one described for the synthesis of Example 114.

MS (ES) MH⁺: 365.2 for C₁₆H₁₇ClN₄O₄.

¹H NMR (400 MHz, DMSO) δ: 0.95 (d, 3H), 1.2 (d, 3H), 2.7-2.9 (m, 2H), 3.2 (d, 1H), 3.4-3.6 (m, 2H), 3.7 (d, 1H), 4.1 (m, 1H), 4.2 (d, 1H), 6.9 (s, 1H), 7.6 (s, 1H), 8.3 (s, 1H).

### Example 116

### (6aS,7S,9R)-rel-7,9-dimethyl-3-(thiophen-2-yl)-6a,7,9,10-tetrahydro-1′H,5H-spiro[[1,4]oxazino[4,3-a][1,7]naphthyridine-6,5′-pyrimidine]-2′,4′,6′(3′H)-trione

1,4-Dioxane (2 ml) and water (400 μl) were de-aerated by bubbling Ar gas through for 20 minutes. The solution was added via syringe to a mixture of (6aS,7S,9R)-rel-3-bromo-7,9-dimethyl-6a,7,9,10-tetrahydro-1′H,5H-spiro[[1,4]oxazino[4,3-a][1,7]naphthyridine-6,5′-pyrimidine]-2′,4′,6′(3′H)-trione (Example 3, 120 mg, 0.29 mmol), thiophen-2-ylboronic acid (37.5 mg, 0.29 mmol), Pd(Ph3P)₄ (33.9 mg, 0.03 mmol), and cesium carbonate (143 mg, 0.44 mmol) under Ar with continual de-aerating by bubbling through Ar for 20 minutes. The mixture was then heated at 100° C. for 3 hours in a microwave reactor. The mixture was diluted with EtOAc and washed with water and brine. Combined aqueous layers were extracted again with EtOAc, which was washed with brine. Drying (MgSO₄) combined EtOAc layers and removal of solvent gave a solid that was chromatographed on silica gel (100% CH₂Cl₂ followed by gradient elution to 70% EtOAc in CH₂Cl₂) to give a solid. The solid was triturated with CH₂Cl₂ to give 22 mg of product as a white solid.

MS (ES) MH⁺: 413 for C₂₀H₂₀N₄O₄S

¹H NMR: 0.95 (d, 3H), 1.15 (d, 3H), 2.85 (m, 1H), 3.0 (d, 1H), 3.5 (m, 1H), 3.7 (m, 2H), 4.1 (d, 1H), 7.0 (s, 1H), 7.4 (s, 4H), 8.2 (s, 1H), 11.6 (s, 1H), 11.8 (s, 1H)

### Example 117

### (6aR,7R,9S)-7,9-Dimethyl-3-(1H-pyrazol-5-yl)-5,6a,7,8,9,10-hexahydro-1′H-spiro[pyrido[1,2-a][1,7]naphthyridine-6,5′-pyrimidine]-2′,4′,6′(3′H)-trione

3-Bromo-7,9-dimethyl-5,6a,7,8,9,10-hexahydro-1′H-spiro[pyrido[1,2-a][1,7]naphthyridine-6,5′-pyrimidine]-2′,4′,6′(3′H)-trione (Example 2) and 5-(4,4,5,5-tetramethyl-1,3,2-dioxaborolan-2-yl)-1H-pyrazole were reacted using a procedure similar to the one described for the synthesis of Example 116, providing the title compound as a 4:1 ratio of diastereomers.

MS (ES) MH⁺: 395 for C₂₀H₂₂N₆O₃.

¹H NMR: 0.7-1.8 (m, 8H), 2.7-3.3 (m, 3H), 4.0-4.2 (m, 2H), 6.6 (2s, 1H), 7.4 (m, 1H), 7.6 (m, 1H), 7.7 (m, 1H), 8.1 (2s, 1H), 11.4 (2s, 1H, 4:1 ratio), 11.6 (2s, 1H, 4:1 ratio), 12.8 (s, broad, 1H).

### Example 118

### (6aS,7S,9R)-rel-7,9-Dimethyl-3-(1H-pyrazol-5-yl)-6a,7,9,10-tetrahydro-2′H,5H-spiro[1,4-oxazino[4,3-a][1,7]naphthyridine-6,5′-pyrimidine]-2′,4′,6′(1′H,3′H)-trione

1,4-Dioxane (2 ml) and water (400 μl) were dearated by bubbling Ar gas trough for 20 minutes. The solution was added via syringe to a mixture of (6aS,7S,9R)-rel-3-bromo-7,9-dimethyl-6a,7,9,10-tetrahydro-1′H,5H-spiro[[1,4]oxazino[4,3-a][1,7]naphthyridine-6,5′-pyrimidine]-2′,4′,6′(3′H)-trione (Example 3, 200 mg, 0.49 mmol) 1-(tetrahydro-2H-pyran-2-yl)-1H-pyrazol-5-ylboronic acid (115 mg, 0.59 mmol), Pd(Ph₃P)₄ (56.5 mg, 0.05 mmol), and Cesium Carbonate (239 mg, 0.73 mmol) under Ar with continual deareating by bubbling through Ar for 20 minutes. The mixture was then heated at 100° C. for 3 hours in a microwave reactor. Solvent was removed and the residue was taken up in 20 ml THF and 20 ml 5N HCl. After stirring at room temperature for 3 hours, the mixture was diluted with EtOAc and washed with aqueous NaHCO₃ and brine. Combined aqueous layers were extracted again with EtOAc, which was washed with brine. Drying (MgSO₄) combined EtOAc layers and removal of solvent gave a solid that was chromatographed on silica gel (100% EtOAc followed by gradient elution to 20% MeOH in EtOAc). Lower Rf material was collected, concentrated and taken up in EtOAc. The EtOAc was filtered and the solid residue was triturated with CH₂Cl₂ to give 20 mg of product as a white solid.

MS (ES) MH¹: 397 for C₁₉H₂₀N₆O₄.

¹H NMR: 0.7-1.8 (m, 8H), 2.7-3.3 (m, 3H), 4.0-4.2 (m, 2H), 6.6 (2s, 1H), 7.4 (m, 1H), 7.6 (m, 1H), 7.7 (m, 1H), 8.1 (2s, 1H), 11.4 (2s, 1H, 4:1 ratio), 11.6 (2s, 1H, 4:1 ratio), 12.8 (s, broad, 1H).

### Example 119

### (6aS,7S,9R)-rel-2-Methoxy-7,9-dimethyl-3-(5-methyl-1,3,4-thiadiazol-2-yl)-6a,7,9,10-tetrahydro-1′H,5H-spiro[[1,4]oxazino[4,3-a][1,8]naphthyridine-6,5′-pyrimidine]-2′., 4′,6′(3′H)-trione

(6aS,7S,9R)-rel-2-Fluoro-7,9-dimethyl-3-(5-methyl-1,3,4-thiadiazol-2-yl)-6a,7,9,10-tetrahydro-1′H,5H-spiro[[1,4]oxazino[4,3-a][1,8]naphthyridine-6,5′-pyrimidine]-2′,4′,6′(3′H)-trione (Example 16, 0.029 g, 0.06 mmol) and 0.5 M NaOH in MeOH (0.390 mL, 0.19 mmol) were combined in MeOH (3 mL) and heated in the microwave at 80° C. for 3 hours. LCMS indicates the reaction is complete. A needle-like solid precipitated from the reaction mixture and that solid was filtered, washed with MeOH and dried (0.018 g, 60% yield).

MS (ES) MH⁺: 459 for C₂₀H₂₂N₆O₅S. ¹H NMR: 0.9 (d, 3H), 1.2 (d, 3H), 2.7 (s, 3H), 2.8 (m, 3H), 3.5 (m, 1H), 4.0 (s, 3H), 5.0 (d, 1H), 7.9 (s, 1H), 9.7 (s, 1H), 10.0 (s, 1H.

### Example 120

A solution of 1,3-di-tert-butyl-5-({2-[(2R,6S)-2,6-dimethylmorpholin-4-yl]-5-(2H-tetrazol-5-yl)pyridin-3-yl}methylidene)pyrimidine-2,4,6(1H,3H,5H)-trione (Intermediate 155, 0.2 g, 0.56 mmol) and methyl iodide (1.57 g, 11.2 mmol) in dichloromethane was stirred for 48 hours at ambient temperature. The dichloromethane was removed and the residue was chromatographed on silica gel (100% CHCl₃ gradient to 1% methanol) to afford 100 mg of a residue that was taken up in acetic acid (5 ml) along with ZnCl₂ (0.04 g, 0.3 mmol) and heated in a sealed tube for 14 hours at 120° C. Acetic acid was removed under reduced pressure and residue was subjected to prep-TLC to give two isomeric compounds: 15 mg of Example 120(a) and 5 mg of Example 120(b).

### Example 120(a)

### (6aR,7R,9S)-rel-7,9-Dimethyl-3-(2-methyl-2H-tetrazol-5-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

MS (ES) MH⁺: 413 for C₁₈H₂₀N₈O₄.

¹H NMR (400 MHz, D₂O) δ: 1.0 (d, 3H), 1.2 (d, 3H), 2.7 (q, 1H), 2.9 (d, 1H), 3.2 (s, 1H), 3.55 (m, 2H), 4.0 (d, 1H), 4.4 (s, 3H), 5.1 (q, 1H), 7.7 (s, 1H), 8.6 (d, 1H)

### Example 120(b)

### (6aR,7R,9S)-rel-7,9-Dimethyl-3-(1-methyl-1H-tetrazol-5-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

MS (ES) MH⁺: 413 for C₁₈H₂₀N₈O₄.

¹H NMR (400 MHz, D₂O) δ: 0.9 (d, 3H), 1.15 (d, 3H), 2.7 (q, 1H), 2.9 (d, 1H), 3.2 (d, 1H), 3.55 (m, 2H), 4.0 (d, 1H), 4.1 (s, 3H), 5.1 (q, 1H), 7.6 (s, 1H), 8.4 (d, 1H).

### Example 121

### (6aS,7S,9R)-rel-N-Hydroxy-7,9-dimethyl-2′,4′,6′-trioxo-1′,3′,4′,6′,6a,7,9,10-octahydro-2′H,5H-spiro[1,4-oxazino[4,3-a][1,8]naphthyridine-6,5′-pyrimidine]-3-carboximidamide

(6aS,7S,9R)-rel-7,9-Dimethyl-2′,4′,6′-trioxo-1′,3′,4′,6′,6a,7,9,10-octahydro-2′H,5H-spiro[1,4-oxazino[4,3-a][1,8]naphthyridine-6,5′-pyrimidine]-3-carbonitrile (Example 113, 2 g, 5.6 mmol), hydroxylamine hydrochloride/methoxyamine hydrochloride (33.8 mmol), and sodium bicarbonate (2.8 g, 33.8 mmol) were dissolved in methanol (60 ml) and the solution was stirred for 14 hours under nitrogen. The solution was cooled and the precipitate was filtered. The residue was dissolved in acetic acid and filtered and the filtrate was concentrated to afford the title product as a white solid. Yield: 1 g (45%).

MS (ES) MH⁺: 389.2 for C₁₇H₂₀N₆O₅.

¹H NMR (400 MHz, D₂O) δ: 1.0 (d, 3H), 1.25 (d, 3H), 2.7 (t, 1H), 2.9 (d, 1H), 3.3-3.9 (m, 2H), 4.2 (d, 1H), 5.0 (d, 1H), 5.7 (s, 2H), 7.4 (s, 1H), 8.2 (d, 1H); 9.4 (s, 1H); 11.8 (br, 2H).

### Example 122

### (6aS,7S,9R)-rel-7,9-dimethyl-3-(5-methyl-1,2,4-oxadiazol-3-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

To a solution of (6aS,7S,9R)-rel-N-hydroxy-7,9-dimethyl-2′,4′,6′-trioxo-1′,3′,4′,6′,6a,7,9,10-octahydro-2′H,5H-spiro[1,4-oxazino[4,3-a][1,8]naphthyridine-6,5′-pyrimidine]-3-carboximidamide (Example 121, 250 mg, 0.64 mmol) in dioxane was added acetic anhydride (0.5 ml, 3.2 mmol) and the solution was heated at 100° C. for 14 hours. The solvent was evaporated and the residue was purified by preparative HPLC to afford the title product as an acetate salt. Yield: 50 mg (20%).

MS (ES) MH⁺: 413.2 for C₁₉H₂₀N₆O₅.

¹H NMR (400 MHz, DMSO) δ: 1.0 (d, 3H), 1.2 (d, 3H), 2.6 (s, 3H), 2.7 (m, 1H), 2.9 (d, 1H), 3.5 (m, 3H), 3.9 (d, 2H), 5.1 (d, 1H), 7.7 (s, 1H), 8.5 (s, 1H), 11.8 (s, broad, 2H).

### Example 123

### (6aS,7S,9R)-rel-7,9-Dimethyl-3-(5-phenyl-1,2,4-oxadiazol-3-yl)-6a,7,9,10-tetrahydro-2′H,5H-spiro[1,4-oxazino[4,3-a][1,8]naphthyridine-6,5′-pyrimidine]-2′,4′,6′(1′H,3′H)-trione

The title compound was synthesized from Example 121 and benzoic anhydride using a procedure similar to the one described for the synthesis of Example 122.

MS (ES) MH⁺: 475.2 for C₂₄H₂₂N₆O₅.

¹H NMR (400 MHz, DMSO) δ: 1.0 (d, 3H), 1.2 (d, 3H), 2.7 (m, 1H), 2.9 (d, 1H), 3.5 (m, 3H), 4.0 (d, 2H), 5.1 (d, 1H), 7.6-7.8 (m, 3H), 8.2 (d, 2H), 8.6 (s, 1H), 11.6 (s, 1H), 11.9 (s, 1H).

1. A compound of Formula (I): 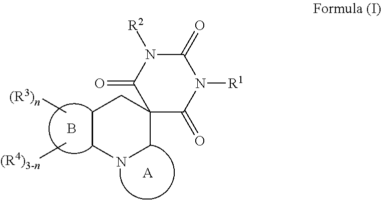
or a pharmaceutically acceptable salt thereof, wherein:
Ring A is a 5- to 7-membered non-aromatic heterocyclic ring, wherein
1) said 5- to 7-membered non-aromatic heterocyclic ring optionally contains, in addition to the nitrogen, a member selected from —O—, —NH—, —S—, —S(O)—, and —S(O)₂—;
2) said 5- to 7-membered non-aromatic heterocyclic ring is optionally substituted on carbon with one or more R⁷;
3) two R⁷ substituents on one carbon atom may together optionally form the group ═O or the group ═N(OR^{7a}); and
4) any —NH— moiety said 5- to 7-membered heterocyclic ring is optionally substituted with R⁷*;
Ring B is a 5- or 6-membered aromatic heterocyclic ring;
n is 0 to 3;
R¹ is selected from H, carbocyclyl, heterocyclyl, —C(O)—H, —C(O)—R^{1b}, —C(O)₂R^{1c}, —C(O)—N(R^{1a})₂, —S(O)—R^{1b}, —S(O)₂—R^{1b}, —S(O)₂—N(R^{1a})₂, —C(R^{1a})═N—R^{1a}, and —C(R^{1a})═N—OR^{1a}, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1a} in each occurrence is independently selected from H, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R¹⁰, and wherein any —NH— moiety of said heterocyclyl is optionally substituted with R¹⁰*;
R^{1c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R¹⁰, and wherein any —NH— moiety of said heterocyclyl is optionally substituted with R¹⁰*;
R² is selected from H, carbocyclyl, heterocyclyl, —C(O)—H, —C(O)—R^{2b}, —C(O)₂R^{2c}, —C(O)—N(R^{2a})₂, —S(O)—R^{2b}, —S(O)₂—R^{2b}, —S(O)₂—N(R^{2a})₂, —C(R^{2a})═N—R^{2a}, and —C(R^{2a})═N—OR^{2a}, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
R^{2a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
R^{2b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R²⁰, and wherein any —NH— moiety of said heterocyclyl is optionally substituted with R²⁰*;
R^{2c} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R²⁰, and wherein any —NH— moiety of said heterocyclyl is optionally substituted with R²⁰*;
R³ in each occurrence is independently selected from —X—R⁵, —W—R⁶, —C(O)—N(R^{3a})—S(O)₂—R^{3b}, —C(R^{3a})═N—R^{3y}, —C(R^{3a})═N—NR^{3a}—C(O)—R^{3b}, —C(N(R^{3a})₂)═N—R^{3y}, —C(N(R^{3a})₂)═N—OR^{3y}, —C(N(R^{3a})₂)═N—C(O)—R^{3b}, —C(N(R^{3a})₂)═N—S(O)₂—R^{3b}, —C(N(R^{3a})₂)═N—CN, —N═C(R^{3y})₂, —N(R^{3a})—S(O)₂—N(R^{3y})₂, —N(R^{3a})—N(R^{3y})₂, —N(R^{3a})—C(O)—N(R^{3y})₂, —N(R^{3a})—C(O)—N(R^{3a})—S(O)₂—R^{3b}, —N(R^{3a})—C(R^{3a})═N(R^{3y}), —N(R^{3a})—C(R^{3a})═N—OR^{3y}, —N(R^{3a})—C(R^{3a})═N—C(O)—R^{3b}, —N(R^{3a})—C(R^{3a})═N—S(O)₂R^{3b}, —N(R^{3a})—C(R^{3a})═N—CN, —N(R^{3a})—C(N(R^{3a})₂)═N—R^{3y}, —N(R^{3a})—C(N(R^{3a})₂)═N—OR^{3y}, —N(R^{3a})—C(N(R^{3a})₂)═N—C(O)—R^{3b}, —N(R^{3a})—C(N(R^{3a})₂)═N—S(O)₂—R^{3b}, —N(R^{3a})—C(N(R^{3a})₂)═N—CN, —O—C(O)—R^{3b}, and —Si(R^{3b})₃;
R^{3a} and R^{3y} in each occurrence are independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R³⁰, and wherein any —NH— moiety of said heterocyclyl is optionally substituted with R³⁰*;
R^{3b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R³⁰, and wherein any —NH— moiety of said heterocyclyl is optionally substituted with R³⁰*;
R⁴ in each occurrence is independently selected from H, halo, —CN, C₁₋₆alkyl, C₂₋₆alkenyl, C₂₋₆alkynyl, carbocyclyl, heterocyclyl, OR^{4d}, —SR^{4d}, —N(R^{4d})₂, —N(R^{4a})—C(O)—R^{4e}, —NO₂, —C(O)—H, —C(O)—R^{4e}, —C(O)₂R^{4d}, —C(O)—N(R^{4d})₂, —O—C(O)—N(R^{4d})₂, —N(R^{4a})—C(O)₂R^{4d}, —S(O)—R^{4e}, —S(O)₂—R^{4e}, —S(O)₂—N(R^{4d})₂, —N(R^{4a})—S(O)₂—R^{4e}, and —C(R^{4a})═N—OR^{4d}, wherein said C₁₋₆alkyl, C₂₋₆alkenyl, and C₂₋₆alkynyl in each occurrence are optionally and independently substituted with one or more R^{40x}, and wherein said carbocyclyl and heterocyclyl in each occurrence are optionally and independently substituted on carbon with one or more R⁴⁰, and wherein any —NH— moiety of said heterocyclyl is optionally substituted with R⁴⁰*;
R^{40a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁴⁰, and wherein any —NH— moiety of said heterocyclyl is optionally substituted with R⁴⁰*;
R^{4d} in each occurrence is independently selected from H, C₁₋₆alkyl, carbocyclyl, and aromatic heterocyclyl, wherein said C₁₋₆alkyl, carbocyclyl, and aromatic heterocyclyl in each occurrence are optionally and independently substituted on carbon with one or more R⁴⁰, and wherein any —NH— moiety of said aromatic heterocyclyl is optionally substituted with R⁴⁰*;
R^{40e} in each occurrence is selected from C₁₋₆alkyl, C₂₋₆alkenyl, C₂₋₆alkynyl, carbocyclyl, and aromatic heterocyclyl, wherein said C₁₋₆alkyl, C₂₋₆alkenyl, C₂₋₆alkynyl, carbocyclyl, and aromatic heterocyclyl in each occurrence are optionally and independently substituted on carbon with one or more R⁴⁰, and wherein any —NH— moiety of said aromatic heterocyclyl is optionally substituted with R⁴⁰*;
R⁵ is selected from heterocyclyl and —Si(R^{5b})₃, wherein said heterocyclyl is optionally substituted on carbon with one or more R⁵⁰, and wherein any —NH— moiety of said heterocyclyl is optionally substituted with R⁵⁰*;
R^{5b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁴⁰, and wherein any —NH— moiety of said heterocyclyl is optionally substituted with R⁵⁰*;
R⁶ is non-aromatic heterocyclyl, wherein said non-aromatic heterocyclyl is optionally substituted on carbon with one or more R⁶⁰, and wherein any —NH— moiety of said non-aromatic heterocyclyl is optionally and independently substituted with R⁶⁰*;
R⁷ is selected from halo, —CN, C₁₋₆alkyl, C₂₋₆alkenyl, C₂₋₆alkynyl, carbocyclyl, heterocyclyl, —OR^{7a}, —SR^{7a}, —N(R^{7a})₂, —N(R^{7a})—C(O)—R^{7b}, —N(R^{7a})—N(R^{7a})₂, —NO₂, —C(O)—H, —C(O)R^{7b}, —C(O)₂R^{7a}, —C(O)—N(R^{7a})₂, —O—C(O)—N(R^{7a})₂, —N(R^{7a})—C(O)₂R^{7a}, —N(R^{7a})—C(O)—N(R^{7a})₂, —O—C(O)—R^{7b}, —S(O)—R^{7b}, —S(O)₂—R^{7b}, —S(O)₂—N(R^{7a)}₂, —N(R^{7a})—S(O)₂—R^{7b}, —C(R^{7a})═N—R^{7a}, and —C(R^{7a})═N—OR^{7a}, wherein said C₁₋₆alkyl, C₂₋₆alkenyl, C₂₋₆alkynyl, carbocyclyl, and heterocyclyl are optionally substituted on carbon with one or more R⁷⁰, and wherein any —NH— moiety of said heterocyclyl is optionally substituted with R⁷⁰*;
R⁷* in each occurrence is independently selected from C₁₋₆alkyl, carbocyclyl, heterocyclyl, —C(O)—H, —C(O)—R^{7b}, —C(O)₂R^{7c}, —C(O)—N(R^{7a})₂, —S(O)—R^{7b}, —S(O)₂—R^{7b}, —S(O)₂—N(R^{7a})₂, —C(R^{7a})═N—R^{7a}, and —C(R^{7a})═N—OR^{7a}, wherein said C₁₋₆alkyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
R^{7a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
R^{7b} in each occurrence is selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
R^{7c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R⁷⁰, and wherein any —NH— moiety of said heterocyclyl is optionally substituted with R⁷⁰*;
R¹⁰ in each occurrence is independently selected from halo, —CN, C₁₋₆alkyl, C₂₋₆alkenyl, C₂₋₆alkynyl, carbocyclyl, heterocyclyl, —OR^{10a}, —SR^{10a}, —N(R^{10a})₂, —N(R^{10a})—C(O)—R^{10b}, —N(R^{10a})—N(R^{10a})₂, —NO₂, —C(O)—H, —C(O)—R^{10b}, —C(O)₂R^{10a}, —C(O)—N(R^{10a})₂, —O—C(O)—N(R^{10a})₂, —N(R^{10a})—C(O)₂R^{10a}, —N(R^{10a})—C(O)—N(R^{10a})₂, —O—C(O)—R^{10b}, —S(O)—R^{10b}, —S(O)₂—R^{10b}, —S(O)₂—N(R^{10a})₂, —N(R^{10a})—S(O)₂—R^{10b}, —C(R^{10a})═N—R^{10a}, and —C(R^{10a})═N—OR^{10a}, wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein any —NH— moiety of said heterocyclyl is optionally substituted with R^{a}*;
R¹⁰* in each occurrence is independently selected from C₁₋₆alkyl, carbocyclyl, heterocyclyl, —C(O)—H, —C(O)—R^{10b}, —C(O)₂R^{10c}, —C(O)—N(R^{10a})₂, —S(O)R^{10b}, —S(O)₂R^{10b}, —S(O)₂—N(R^{10a})₂, —C(R^{10a})═N—R^{10a}, and —C(R^{10a})═N—OR^{10a}, wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10a} in each occurrence is independently selected from H, carbocyclyl, and heterocyclyl, wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{a}, and wherein any —NH— moiety of said heterocyclyl is optionally substituted with R^{a}*;
R^{10c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{a}, and wherein any —NH— moiety of said heterocyclyl is optionally substituted with R^{a}*;
R²⁰ in each occurrence is independently selected from halo, —CN, C₂₋₆alkenyl, C₂₋₆alkynyl, carbocyclyl, heterocyclyl, —OR^{20a}, —SR^{20a}, —N(R^{20a})₂, —N(R^{20a})—C(O)—R^{20b}, —N(R^{20a})—N(R^{20a})₂, —NO₂, —C(O)—H, —C(O)—R^{20b}, —C(O)₂R^{20a}, —C(O)—N(R^{20a})₂, —O—C(O)—N(R^{20a})₂, —N(R^{20a})—C(O)₂R^{20a}, —N(R^{20a})—C(O)—N(R^{20a})₂, —O—C(O)—R^{20b}, —S(O)—R^{20b}, —S(O)₂—R^{20b}, —S(O)₂—N(R^{20a})₂, —N(R^{20a})—S(O)₂—R^{20b}, —C(R^{20a})═N—R^{20a}, and —C(R^{20a})═N—OR^{20a}, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R²⁰* in each occurrence is independently selected from C₁₋₆alkyl, carbocyclyl, heterocyclyl, —C(O)—H, —C(O)—R^{20b}, —C(O)₂R^{20c}, —C(O)—N(R^{20a})₂, —S(O)—R^{20b}, —S(O)₂—R^{20b}, —S(O)₂—N(R^{20a})₂, —C(R^{20a})═N—R^{20a}, and —C(R^{20a})═N—OR^{20a}, wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R^{20c} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{b}, and wherein any —NH— moiety of said heterocyclyl is optionally substituted with R^{b}*;
R³⁰ in each occurrence is independently selected from halo, —CN, C₁₋₆alkyl, C₂₋₆alkenyl, C₂₋₆alkynyl, carbocyclyl, heterocyclyl, —OR^{30a}, —SR^{30a}, —N(R^{30a})₂, —N(R^{30a})—C(O)—R^{30b}, —N(R^{30a})—N(R^{30a})₂, —NO₂, —C(O)—H, —C(O)—R^{30b}, —C(O)₂R^{3a}, —C(O)—N(R^{30a})₂, —O—C(O)—N(R^{30a})₂, —N(R^{30a})—C(O)₂R^{30a}, —N(R^{30a})—C(O)—N(R^{30a})₂, —O—C(O)—R^{30b}, —S(O)—R^{30b}, —S(O)₂—R^{30b}, —S(O)₂—N(R^{30a})₂, —N(R^{30a})—S(O)₂—R^{30b}, —Si(R^{30b})₃, —C(R^{30a})═N—R^{30a}, and —C(R^{30a})═N—OR^{30a}, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein any —NH— moiety of said heterocyclyl is optionally substituted with R^{c}*;
R³⁰* in each occurrence is independently selected from C₁₋₆alkyl, carbocyclyl, heterocyclyl, —C(O)—H, —C(O)—R^{30b}, —C(O)₂R^{30c}, —C(O)—N(R^{30a})₂, —S(O)—R^{30b}, —S(O)₂—R^{30b}, —S(O)₂—N(R^{30a})₂, —C(R^{30a})═N—R^{30a}, and —C(R^{30a})═N—OR^{30a}, wherein said C₁₋₆alkyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein any —NH— moiety of said heterocyclyl is optionally substituted with R^{c}*;
R^{30c} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{c}, and wherein any —NH— moiety of said heterocyclyl is optionally substituted with R^{c}*;
R⁴⁰ in each occurrence is independently selected from halo, —CN, C₁₋₆alkyl, C₂₋₆alkenyl, C₂₋₆alkynyl, carbocyclyl, heterocyclyl, —OR^{40a}, —SR^{40a}, —N(R^{40a)}₂, —N(R^{40a})—C(O)—R^{40b}, —N(R^{40a})—N(R^{40a)}₂, —NO₂, —C(O)—H, —C(O)—R^{40b}, —C(O)₂R^{40a}, —C(O)—N(R^{40a})₂, —O—C(O)—N(R^{40a})₂, —N(R^{40a})—C(O)₂R^{40a}, —N(R^{40a})—C(O)—N(R^{40a})₂, —O—C(O)—R^{40b}, —S(O)—R^{40b}, —S(O)₂—R^{40b}, —S(O)₂—N(R^{40a})₂, —N(R^{40a})—S(O)₂—R^{40b}, —C(R^{40a})═N—R^{40a}, and —C(R^{40a})═N—OR^{40a}, wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein any —NH— moiety of said heterocyclyl is optionally substituted with R^{d}*;
R⁴⁰* in each occurrence is independently selected from C₁₋₆alkyl, carbocyclyl, heterocyclyl, —C(O)—H, —C(O)—R^{40b}, —C(O)₂R^{40c}, —C(O)—N(R^{40a})₂, —S(O)—R^{40b}, —S(O)₂—R^{40b}, —S(O)₂—N(R^{40a})₂, —C(R^{40a})═N—R^{40a}, and —C(R^{40a})═N—OR^{40a}, wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{d}, and wherein any —NH— moiety of said heterocyclyl is optionally substituted with R^{d}*;
R^{40x} in each occurrence is independently selected from halo, —CN, C₁₋₆alkyl, C₂₋₆alkenyl, C₂₋₆alkynyl, carbocyclyl, —OR^{40a}, —SR^{40a}, —N(R^{40a})₂, —N(R^{40a})—C(O)—R^{40b}, —N(R^{40a})—N(R^{40a})₂, —NO₂, —C(O)—H, —C(O)—R^{40b}, —C(O)₂R^{40a}, —C(O)—N(R^{40a})₂, —O—C(O)—N(R^{40a})₂, —N(R^{40a})—C(O)₂R^{40a}, —N(R^{40a})—C(O)—N(R^{40a})₂, —O—C(O)—R^{40b}, —S(O)—R^{40b}, —S(O)₂—R^{40b}, —S(O)₂—N(R^{40a})₂, —N(R^{40a})—S(O)₂—R^{40b}, —C(R^{40a})═N—R^{40a}, and —C(R^{40a})═N—OR^{40a}, wherein said C₁₋₆alkyl, C₂₋₆alkenyl, C₂₋₆alkynyl, and carbocyclyl in each occurrence are optionally and independently substituted on carbon with one or more R^{d};
R⁵⁰ in each occurrence is independently selected from halo, —CN, C₁₋₆alkyl, C₂₋₆alkenyl, C₂₋₆alkynyl, carbocyclyl, heterocyclyl, —OR⁵⁰, —SR^{50a}, —N(R^{50a})₂, —N(R^{50a})—C(O)—R^{50b}, —N(R^{50a})—N(R^{50a})₂, —NO₂, —C(O)—H, —C(O)—R^{50b}, —C(O)₂R^{50a}, —C(O)—N(R^{50a})₂, —O—C(O)—N(R^{50a})₂, —N(R^{50a})—C(O)₂R^{50a}, —N(R^{50a})—C(O)—N(R^{50a})₂, —O—C(O)—R^{50b}, —S(O)—R^{50b}, —S(O)₂—R^{50b}, —S(O)₂—N(R^{50a})₂, —N(R^{50a})—S(O)₂—R^{50b}, —Si(R^{50b})₃, —C(R^{50a})═N(R^{50a}), and —C(R^{50a})═N(OR^{50a}), wherein said C₁₋₆alkyl, C₂₋₆alkenyl, C₂₋₆alkynyl, carbocyclyl, and heterocyclyl in each occurrence are optionally and independently substituted on carbon with one or more R^{e}, and wherein any —NH— moiety of said heterocyclyl is optionally substituted with R^{e}*;
R⁵⁰* in each occurrence is independently selected from C₁₋₆alkyl, carbocyclyl, heterocyclyl, —C(O)—H, —C(O)—R^{50b}, —C(O)₂R^{50c}, —C(O)—N(R^{50a})₂, —S(O)—R^{50b}, —S(O)₂—R^{50b}, —S(O)₂—N(R^{50a})₂, —C(R^{50a})═N—R^{50a}, and —C(R^{50a})═N—OR^{50a}, wherein said C₁₋₆alkyl, carbocyclyl, and heterocyclyl in each occurrence are optionally and independently substituted on carbon with one or more R^{e}, and wherein any —NH— moiety of said heterocyclyl is optionally substituted with R^{e}*;
R^{50a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{e}, and wherein any —NH— moiety of said heterocyclyl is optionally substituted with R^{e}*;
R^{50b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{e}, and wherein any —NH— moiety of said heterocyclyl is optionally substituted with R^{e}*;
R^{50c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{e}, and wherein any —NH— moiety of said heterocyclyl is optionally substituted with R^{e}*;
R⁶⁰ in each occurrence is independently selected from halo, —CN, C₁₋₆alkyl, C₂₋₆alkenyl, C₂₋₆alkynyl, carbocyclyl, heterocyclyl, —OR^{60a}, —SR^{60a}, —N(R^{60a})₂, —N(R^{60a})—C(O)—R^{60b}, —N(R^{60a})—N(R^{60a})₂, —NO₂, —C(O)—H, —C(O)—R^{60b}, —C(O)₂R^{60a}, —C(O)—N(R^{60a})₂, —O—C(O)—N(R^{60a})₂, —N(R^{60a})—C(O)₂R^{60a}, —N(R^{60a})—C(O)—N(R^{60a})₂, —O—C(O)—R^{60b}, —S(O)—R^{60b}, —S(O)₂—R^{60b}, —S(O)₂—N(R^{60a})₂, —N(R^{60a})—S(O)₂—R^{60b}, —C(R^{60a})═N—R^{60a}, and —C(R^{60a})═N—OR^{60a}, wherein said C₁₋₆alkyl, C₂₋₆alkenyl, C₂₋₆alkynyl, carbocyclyl, and heterocyclyl in each occurrence are optionally and independently substituted on carbon with one or more R^{f}, and wherein any —NH— moiety of said heterocyclyl is optionally substituted with R^{f}*;
R⁶⁰* in each occurrence is independently selected from C₁₋₆alkyl, carbocyclyl, heterocyclyl, —C(O)—H, —C(O)—R^{60b}, —C(O)₂R^{60c}, —C(O)—N(R^{60a})₂, —S(O)—R^{60b}, —S(O)₂—R^{60b}, —S(O)₂—N(R^{60a})₂, —C(R^{60a})═N—R^{60a}, and —C(R^{60a})═N—OR^{60a}, wherein said C₁₋₆alkyl, carbocyclyl, and heterocyclyl in each occurrence are optionally and independently substituted on carbon with one or more R^{f}, and wherein any —NH— moiety of said heterocyclyl is optionally substituted with R^{f}*;
R^{60a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein any —NH— moiety of said heterocyclyl is optionally substituted with R^{f}*;
R^{60b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{f}, and wherein any —NH— moiety of said heterocyclyl is optionally substituted with R^{f}*;
R^{60c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{f}, and wherein any —NH— moiety of said heterocyclyl is optionally substituted with R^{f}*;
R⁷⁰ in each occurrence is independently selected from halo, —CN, C₁₋₆alkyl, C₂₋₆alkenyl, C₂₋₆alkynyl, carbocyclyl, heterocyclyl, —OR^{70a}, —SR^{70a}—N(R^{70a})₂, —N(R^{70a})—C(O)—R^{70b}, —N(R^{70a})—N(R^{70a})₂, —NO₂, —C(O)—H, —C(O)—R^{70b}, —C(O)₂R^{70a}, —C(O)—N(R^{70a})₂, —O—C(O)—N(R^{70a})₂, —N(R^{70a})—C(O)₂R^{70a}, —N(R^{70a})—C(O)—N(R^{70a})₂, —O—C(O)—R^{70b}, —S(O)—R^{70b}, —S(O)₂—R^{70b}, —S(O)₂—N(R^{70a})₂, —N(R^{70b})—S(O)₂—R^{70b}, —C(R^{70a})═N—R^{70a}, and —C(R^{70a})═N—OR^{70a}, wherein said C₁₋₆alkyl, C₂₋₆alkenyl, C₂₋₆alkynyl, carbocyclyl, and heterocyclyl in each occurrence are optionally and independently substituted on carbon with one or more R^{g}, and wherein any —NH— moiety of said heterocyclyl is optionally substituted with R^{g}*;
R⁷⁰* in each occurrence is independently selected from C₁₋₆alkyl, carbocyclyl, heterocyclyl, —C(O)—H, —C(O)—R^{70b}, —C(O)₂R^{70c}, —C(O)—N(R^{70a})₂, —S(O)—R^{70b}, —S(O)₂—R^{70b}, —S(O)₂—N(R^{70a})₂, —C(R^{70a})═N—R^{70a}, and —C(R^{70a})═N—OR^{70a}, wherein said C₁₋₆alkyl, carbocyclyl, and heterocyclyl in each occurrence are optionally and independently substituted on carbon with one or more R^{g}, and wherein any —NH— moiety of said heterocyclyl is optionally substituted with R^{g}*;
R^{70a} in each occurrence is independently selected from H, C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein any —NH— moiety of said heterocyclyl is optionally substituted with R^{g}*;
R^{70b} in each occurrence is independently selected from C₁₋₆alkyl, C₂₋₆alkenyl, C₂₋₆alkynyl, carbocyclyl, and heterocyclyl, wherein said C₁₋₆alkyl, C₂₋₆alkenyl, C₂₋₆alkynyl, carbocyclyl, and heterocyclyl in each occurrence are optionally and independently substituted on carbon with one or more R^{g}, and wherein any —NH— moiety of said heterocyclyl is optionally substituted with R^{g}*;
R^{70c} in each occurrence is independently selected from C₁₋₆alkyl, carbocyclyl, and heterocyclyl, wherein said C₁₋₆alkyl, carbocyclyl, and heterocyclyl in each occurrence are optionally and independently substituted on carbon with one or more R^{g}, and wherein any —NH— moiety of said heterocyclyl is optionally substituted with R^{g}*;
R^{a}, R^{b}, R^{c}, R^{d}, R^{e}, R^{f}, and R^{g} in each occurrence are independently selected from halo, —CN, C₁₋₆alkyl, C₂₋₆alkenyl, C₂₋₆alkynyl, carbocyclyl, heterocyclyl, —OR^{m}, —SR^{m}, —N(R^{m})₂, —N(R^{m})—C(O)—Rⁿ, —N(R^{m})—N(R^{m})₂, —NO₂, —C(O)—H, —C(O)—Rⁿ, —C(O)₂R^{m}, —C(O)—N(R^{m})₂, —O—C(O)—N(R^{m})₂, —N(R^{m})—C(O)₂R^{m}, —N(R^{m})—C(O)—N(R^{m})₂, —O—C(O)—Rⁿ, —S(O)—Rⁿ, —S(O)₂—Rⁿ, —S(O)₂—N(R^{m})₂, —N(R^{m})—S(O)₂—Rⁿ, —C(R^{m})═N—R^{m}, and —C(R^{m})═N—OR^{m};
R^{a}*, R^{b}*, R^{c}*, R^{d}, R^{e}*, R^{f}*, and R^{g} in each occurrence are independently selected from C₁₋₆alkyl, carbocyclyl, heterocyclyl, —C(O)—H, —C(O)—Rⁿ, —C(O)₂R^{o}, —C(O)—N(R^{m})₂, —S(O)—Rⁿ, —S(O)₂—Rⁿ, —S(O)₂—N(R^{m})₂, —C(R^{m})═N—R^{m}, and —C(R^{m})═N—OR^{m};
R^{m} in each occurrence is independently selected from H, carbocyclyl, and heterocyclyl;
Rⁿ in each occurrence is independently selected from C₁₋₆alkyl, C₂₋₆alkenyl, C₂₋₆alkynyl, carbocyclyl, and heterocyclyl;
R^{o} in each occurrence is independently selected from C₁₋₆alkyl, carbocyclyl, and heterocyclyl;
W in each occurrence is independently selected from —O—, —S—, —N(R^{3a})—, —N(R^{3a})C(O)—, —C(O)—, —C(O)₂—, —C(O)—N(R^{3a})—, —O—C(O)—N(R^{3a})—, —N(R^{3a})—C(O)₂—, —S(O)—, —S(O)₂—, —S(O)₂—, and —N(R^{3a})—S(O)₂—; and
X in each occurrence is independently selected from C₁₋₆alkylene, C₂₋₆alkenylene, and C₂₋₆alkynylene, wherein said C₁₋₆alkylene, C₂₋₆alkenylene, and C₂₋₆alkynylene in each occurrence are optionally and independently substituted one or more R⁴⁰.

2. A compound of Formula (I), or a pharmaceutically acceptable salt thereof, as claimed in claim 1, wherein
R¹ and R² are H.

3. A compound of Formula (I), or a pharmaceutically acceptable salt thereof, as claimed in claim 1, wherein
Ring A is a 6-membered non-aromatic heterocyclic ring, wherein
1) said 6-membered non-aromatic heterocyclic ring optionally contains, in addition to the nitrogen, a member selected from —O— and —NH—; and
2) said 6-membered non-aromatic heterocyclic ring is optionally substituted on carbon with one or more R⁷; and
R⁷ is C₁₋₆alkyl.

4. A compound of Formula (I), or a pharmaceutically acceptable salt thereof, as claimed in claim 1, wherein
Ring B is pyridine.

5. A compound of Formula (I), or a pharmaceutically acceptable salt thereof, as claimed in claim 1, wherein
n is 0 or 1;
R³ is selected from —X—R⁵ and —C(NH₂)═N—OH;
R⁵ in each occurrence is independently selected from phenyl and 5- or 6-membered heteroaryl, wherein said phenyl and 5- or 6-membered heteroaryl in each occurrence are optionally and independently substituted with one or more R⁵⁰;
R⁵⁰ is —OR^{50a};
R^{50a} is C₁₋₆alkyl; and
X is ethyne-1,2-diyl.

6. A compound of Formula (I), or a pharmaceutically acceptable salt thereof, as claimed in claim 1, wherein
R⁴ in each occurrence is independently selected from H, —CN, halo, phenyl, 5- or 6-membered heteroaryl, and 9- or 10-membered bicyclic heteroaryl, wherein said phenyl, 5- or 6-membered heteroaryl, and 9- or 10-membered bicyclic heteroaryl in each occurrence are optionally substituted with one or more R⁴⁰, and wherein any —NH— moiety of said 5- or 6-membered heteroaryl is optionally substituted with R⁴⁰*;
R⁴⁰ in each occurrence is independently selected from halo, C₁₋₆alkyl, phenyl, 5- or 6-membered heterocyclyl, —OR^{40a}, and —N(R^{40a})₂;
R⁴⁰* is C₁₋₆alkyl; and
R^{40a} in each occurrence is independently selected from H and C₁₋₆alkyl.

7. A compound of Formula (I): 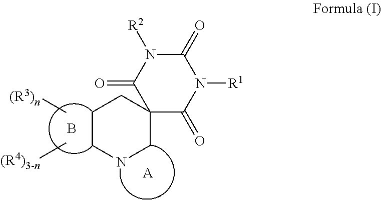
or a pharmaceutically acceptable salt thereof, wherein:
Ring A is selected from 2,6-dimethylmorpholine, 3,5-dimethylpiperidine, 6-methylpiperazin-2-one, and piperidine;
Ring B is pyridine;
n is 0 or 1;
R¹ is H;
R² is H;
R³ is selected from —C(NH₂)═N—OH, 4-methoxyphenylethynyl, and pyrazin-2-ylethynyl; and
R⁴ in each occurrence is independently selected from H, —CN, bromo, chloro, fluoro, iodo, 1H-benzimidazol-2-yl, 1-benzofuran-2-yl, 1,3-benzothiazol-2-yl, 1-benzothien-2-yl, 5-chloropyridin-2-yl, 2-(dimethyylamino)pyrimidin-5-yl, 3,5-dimethylisoxazol-4-yl, 2,4-dimethyl-1,3-thiazol-5-yl, 4-fluorophenyl, furan-2-yl, furan-3-yl, 1H-imidazol-2-yl, 1H-imidazol-4-yl, 2-methoxyphenyl, 3-methyoxypyrazin-2-yl, 6-methoxypyrazin-2-yl, 4-methoxypyridin-3-yl, 2-methoxy-1,3-thiaol-4-yl, 1-methyl-1H-imidazol-2-yl, 1-methyl-1H-imidazol-4-yl, 1-methyl-1H-imidazol-5-yl, 2-methylphenyl, 1-methyl-1H-pyrazol-4-yl, 1-methyl-1H-pyrazol-5-yl, 1-methyl-1H-tetrazol-5-yl, 2-methyl-2H-tetrazol-5-yl, 5-methyl-1,3,4-thiadiazoly-2-yl, 3-methylthiophen-2-yl, 4-methylthiophen-3-yl, 5-methylthiophen-2-yl, 6-(morpholin-4-yl)pyridin-3-yl, 5-methyl-1,2,4-oxathiadiazol-3-yl, 1,3-oxazol-2-yl, phenyl, pyrazin-2-yl, 1H-pyrazol-4-yl, 1H-pyazol-5-yl, 5-(1H-pyrazol-5-yl)thiophen-2-yl, pyridazin-4-yl, pyridin-2-yl, pyridin-3-yl, pyridin-4-yl, pyrimidin-2-yl, pyrimidin-5-yl, quinolin-2-yl, quinolin-8-yl, 1,3,4-thiadiazol-2-yl, 1,3-thiazol-2-yl, 1,3-thiazol-4-yl, thiazol-5-yl, thiophen-2-yl, and 5-(1H-tetrazol-5-yl)thiophen-2-yl.

8-9. (canceled)

10. A method for treating a bacterial infection in a warm-blooded animal such as man, said method comprising administering to said animal an effective amount of a compound of Formula (I), as claimed in claim 1, or a pharmaceutically acceptable salt thereof.

11. (canceled)

12. A pharmaceutical composition comprising a compound of Formula (I), or a pharmaceutically acceptable salt thereof, as claimed in claim 1, and at least one pharmaceutically acceptable carrier, diluent, or excipient.

13. A process for preparing a compound of Formula (I), or a pharmaceutically acceptable salt thereof, as claimed in claim 1, said process comprising reacting a compound of Formula (A1): 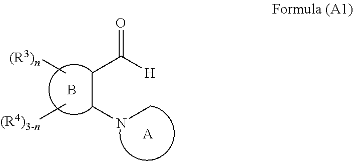
with a compound of Formula (A2): 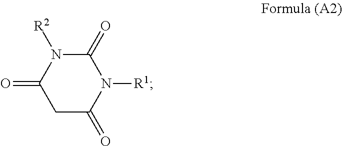
and thereafter if necessary:
i) converting a compound of Formula (I) into another compound of Formula (I);
ii) removing any protecting groups; and/or
iii) forming a pharmaceutically acceptable salt.
